SEQUENCE LISTING (120 sequences)

SEQ ID NO: 1 - protein, 36 aa, Homo sapiens
YPSKPDNPGEDAPAEDMARYYSALRHYINLITRQRY

SEQ ID NO: 2 - protein, 36 aa, Mus musculus; identical to SEQ ID NO: 1

SEQ ID NO: 3 - protein, 36 aa, Homo sapiens
YPIKPEAPGEDASPEELNRYYASLRHYLNLVTRQRY

SEQ ID NO: 4 - protein, 36 aa, Mus musculus
YPAKPEAPGEDASPEELSRYYASLRHYLNLVTRQRY

SEQ ID NO: 5 - protein, 36 aa, Homo sapiens
  MISC_FEATURE (36)..(36): single amide group
APLEPVYPGDNATPEQMAQYAADLRRYINMLTRPRY

SEQ ID NO: 6 - protein, 36 aa, Mus musculus
  MISC_FEATURE (36)..(36): single amide group
APLEPMYPGDYATPEQMAQYETQLRRYINTLTRPRY

SEQ ID NO: 7 - protein, 25 aa, Artificial Sequence
  1p1
APAEDMARYYSALRHYINLITRQRY

SEQ ID NO: 8 - protein, 25 aa, Artificial Sequence
  1p2
APAEDMARAYSALRHYINLITRQRY

SEQ ID NO: 9 - protein, 25 aa, Artificial Sequence
  1p3
APAEDMARYASALRHYINLITRQRY

SEQ ID NO: 10 - protein, 25 aa, Artificial Sequence
  1p4
APAEDMARYYAALRHYINLITRQRY

SEQ ID NO: 11 - protein, 25 aa, Artificial Sequence
  1p5
APAEDMARYYSALRHYINLITRQRY

SEQ ID NO: 12 - protein, 25 aa, Artificial Sequence
  1p6
APAEDMARYYSAARHYINLITRQRY

SEQ ID NO: 13 - protein, 25 aa, Artificial Sequence
  1p7
APAEDMARYYSALAHYINLITRQRY

SEQ ID NO: 14 - protein, 25 aa, Artificial Sequence
  1p8
APAEDMARYYSALRAYINLITRQRY

SEQ ID NO: 15 - protein, 25 aa, Artificial Sequence
  1p9
APAEDMARYYSALRHAINLITRQRY

SEQ ID NO: 16 - protein, 25 aa, Artificial Sequence
  1p10
APAEDMARYYSALRHYANLITRQRY

SEQ ID NO: 17 - protein, 25 aa, Artificial Sequence
  1p11
APAEDMARYYSALRHYIALITRQRY

SEQ ID NO: 18 - protein, 25 aa, Artificial Sequence
  1p12
APAEDMARYYSALRHYINAITRQRY

SEQ ID NO: 19 - protein, 25 aa, Artificial Sequence
  1p13
APAEDMARYYSALRHYINLATRQRY

SEQ ID NO: 20 - protein, 25 aa, Artificial Sequence
  1p14
APAEDMARYYSALRHYINLIARQRY

SEQ ID NO: 21 - protein, 25 aa, Artificial Sequence
  1p15
APAEDMARYYSALRHYINLITAQRY

SEQ ID NO: 22 - protein, 25 aa, Artificial Sequence
  1p16
APAEDMARYYSALRHYINLITRARY

SEQ ID NO: 23 - protein, 25 aa, Artificial Sequence
  1p17
APAEDMARYYSALRHYINLITRQAY

SEQ ID NO: 24 - protein, 25 aa, Artificial Sequence
  1p18
APAEDMARYYSALRHYINLITRQRA

SEQ ID NO: 25 - protein, 10 aa, Artificial Sequence
  1p19
AEDMARYYSA

SEQ ID NO: 26 - protein, 10 aa, Artificial Sequence
  1p20
EDMARYYSAL

SEQ ID NO: 27 - protein, 10 aa, Artificial Sequence
  1p21
DMARYYSALR

SEQ ID NO: 28 - protein, 10 aa, Artificial Sequence
  1p22
MARYYSALRH

SEQ ID NO: 29 - protein, 10 aa, Artificial Sequence
  1p23
ARYYSALRHY

SEQ ID NO: 30 - protein, 10 aa, Artificial Sequence
  1p24
RYYSALRHYI

SEQ ID NO: 31 - protein, 10 aa, Artificial Sequence
  1p25
YYSALRHYIN

SEQ ID NO: 32 - protein, 10 aa, Artificial Sequence
  1p26
YSALRHYINL

SEQ ID NO: 33 - protein, 10 aa, Artificial Sequence
  1p27
SALRHYINLI

SEQ ID NO: 34 - protein, 10 aa, Artificial Sequence
  1p28
ALRHYINLIT

SEQ ID NO: 35 - protein, 10 aa, Artificial Sequence
  1p29
LRHYINLITR

SEQ ID NO: 36 - protein, 10 aa, Artificial Sequence
  1p30
RHYINLITRQ

SEQ ID NO: 37 - protein, 10 aa, Artificial Sequence
  1p31
HYINLITRQR

SEQ ID NO: 38 - protein, 10 aa, Artificial Sequence
  1p32
YINLITRQRY

SEQ ID NO: 39 - protein, 17 aa, Artificial Sequence
  2p1
AYSALRHYINLITRQRY

SEQ ID NO: 40 - protein, 17 aa, Artificial Sequence
  2p2
AASALRHYINLITRQRY

SEQ ID NO: 41 - protein, 17 aa, Artificial Sequence
  2p3
AAAALRHYINLITRQRY

SEQ ID NO: 42 - protein, 17 aa, Artificial Sequence
  2p4
AAAAARHYINLITRQRY

SEQ ID NO: 43 - protein, 17 aa, Artificial Sequence
  2p5
AAAAAAHYINLITRQRY

SEQ ID NO: 44 - protein, 17 aa, Artificial Sequence
  2p6
AAAAAAAYINLITRQRY

SEQ ID NO: 45 - protein, 17 aa, Artificial Sequence
  2p7
AAAAAAAAINLITRQRY

SEQ ID NO: 46 - protein, 17 aa, Artificial Sequence
  2p8
AAAAAAAAANLITRQRY

SEQ ID NO: 47 - protein, 17 aa, Artificial Sequence
  2p9
AAAAAAAAAALITRQRY

SEQ ID NO: 48 - protein, 17 aa, Artificial Sequence
  2p10
AAAAAAAAAAAITRQRY

SEQ ID NO: 49 - protein, 17 aa, Artificial Sequence
  2p11
AAAAAAAAAAAATRQRY

SEQ ID NO: 50 - protein, 17 aa, Artificial Sequence
  2p12
AAAAAAAAAAAATRQRY

SEQ ID NO: 51 - protein, 17 aa, Artificial Sequence
  2p13
AAAAAAAAAAAAARQRY

SEQ ID NO: 52 - protein, 17 aa, Artificial Sequence
  2p14
AAAAAAAAAAAAAAQRY

SEQ ID NO: 53 - protein, 17 aa, Artificial Sequence
  2p15
AAAAAAAAAAAAAAARY

SEQ ID NO: 54 - protein, 17 aa, Artificial Sequence
  2p16
AAAAAAAAAAAAAAAAY

SEQ ID NO: 55 - protein, 21 aa, Artificial Sequence
  2p17
YYSALRHYINLITRQRYAAAA

SEQ ID NO: 56 - protein, 17 aa, Artificial Sequence
  2p18
YETQLRRYINTLTRQRY

SEQ ID NO: 57 - protein, 17 aa, Artificial Sequence
  2p19
YYSALRHYINLITRQRY

SEQ ID NO: 58 - protein, 17 aa, Artificial Sequence
  2p20
GGGGGGGGGGGGGGQRY

SEQ ID NO: 59 - protein, 17 aa, Artificial Sequence
  3p1
AAAAAAAAANTLTRPRY

SEQ ID NO: 60 - protein, 17 aa, Artificial Sequence
  3p2
AAAAAAAAANMLTRPRY

SEQ ID NO: 61 - protein, 17 aa, Artificial Sequence
  3p3
AAAAAAAAANLVTRQRY

SEQ ID NO: 62 - protein, 17 aa, Artificial Sequence
  3p4
AAAAAAAAANLITRQRY

SEQ ID NO: 63 - protein, 17 aa, Artificial Sequence
  3p5
AAAAAAAAAAAAAFMRF

SEQ ID NO: 64 - protein, 17 aa, Artificial Sequence
  3p6
AAAAAAAAAFLFQPQRF

SEQ ID NO: 65 - protein, 18 aa, Artificial Sequence
  3p7
AGEGLSSPFWSLAAPQRF

SEQ ID NO: 66 - protein, 17 aa, Artificial Sequence
  3p8
AAAAAAAAAAAAAPQRF

SEQ ID NO: 67 - protein, 17 aa, Artificial Sequence
  3p9
AAAAAAAAAAAAAPQRY

SEQ ID NO: 68 - protein, 17 aa, Artificial Sequence
  3p10
AAAAAAAAAAAAAANRY

SEQ ID NO: 69 - protein, 17 aa, Artificial Sequence
  3p11
AAAAAAAAAAAAAAERY

SEQ ID NO: 70 - protein, 17 aa, Artificial Sequence
  3p12
AAAAAAAAAAAAAADRY

SEQ ID NO: 71 - protein, 17 aa, Artificial Sequence
  3p13
AAAAAAAAAAAAAARRY

SEQ ID NO: 72 - protein, 17 aa, Artificial Sequence
  3p14
AAAAAAAAAAAAAAKRY

SEQ ID NO: 73 - protein, 17 aa, Artificial Sequence
  3p15
AAAAAAAAAAAAAAQQY

SEQ ID NO: 74 - protein, 17 aa, Artificial Sequence
  3p16
AAAAAAAAAAAAAAQKY

SEQ ID NO: 75 - protein, 17 aa, Artificial Sequence
  3p17
AAAAAAAAAAAAAAQRH

SEQ ID NO: 76 - protein, 17 aa, Artificial Sequence
  3p18
AAAAAAAAAAAAAAQRF

SEQ ID NO: 77 - protein, 17 aa, Artificial Sequence
  3p19
AAAAAAAAAAAAAAQRW

SEQ ID NO: 78 - protein, 17 aa, Artificial Sequence
  immunizing peptide
YYSALRHYINLITRQRY

SEQ ID NO: 79 - protein, 3 aa, artificial
  Consensus sequence of epitope
XRY

SEQ ID NO: 80 - protein, 3 aa, Artificial Sequence
  synthetic 1
QRY

SEQ ID NO: 81 - protein, 3 aa, Artificial Sequence
  synthetic peptide 2
PRY

SEQ ID NO: 82 - protein, 4 aa, Artificial Sequence
  synthetic peptide 3
FMRF

SEQ ID NO: 83 - protein, 8 aa, Artificial Sequence
  synthetic peptide 4
FLFQPQRF

SEQ ID NO: 84 - protein, 3 aa, Artificial Sequence
  synthetic peptide 5
QRF

SEQ ID NO: 85 - protein, 112 aa, Artificial Sequence
  amino acid sequence of variable heavy chain 1
QVQLQHPGTEVVKPGASVKLSCKASGYTFTNYWMHWVKQRPGQGLEWIGNIDPSNGGTNYNEKFKSKATLTVDKSSSTAYMQLSSLTSEDSAVYYCASFAYWGQGTLVTVSA

SEQ ID NO: 86 - protein, 112 aa, Artificial Sequence
  amino acid sequence of variable heavy chain 2
QVQLQHPGTEVVRPGASVKLSCKASGYTFTNYWMHWVKQRPGQGLEWIGNIDPSNGGTNYNEKFKSKATLTVDKSSSTAYMQLSSLTSEDSAVYYCASFAYWGQGTLVTVSA

SEQ ID NO: 87 - protein, 107 aa, Artificial Sequence
  amino acid sequence of variable light chain 1
DIQMTQSPASLSASVGETVTITCRASGNIHNYLAWYQQKQGKSPQLLVYNAKTLADGVPSRFSGSGSGTQYSLKISSLRPEDFGSYYCQHFWSPPFTFGSGTKLEIK

SEQ ID NO: 88 - DNA, 339 bp, Artificial Sequence
  nucleotide sequence of variable heavy chain 1
caggtccaactgcagcaccctgggactgaagtggtgaagcctggggcttcagtgaagctgtcctgcaaggcttctggctacaccttcaccaactactggatgcactgggtgaagcagaggcctggacaaggccttgagtggattggaaatattgatcctagcaatggtggtactaactataatgagaagttcaagagcaaggccacactgactgtagacaaatcctccagcacagcctacatgcagctcagcagcctgacatctgaggactctgcggtctattattgtgcaagttttgcttactggggccaagggactctggtcactgtctctgcagcc

SEQ ID NO: 89 - DNA, 336 bp, Artificial Sequence
  nucleotide sequence of variable heavy chain 2
caggtccaactgcagcaccctgggactgaagtggtgaggcctggggcttcagtgaagctgtcctgcaaggcttctggctacaccttcaccaactactggatgcactgggtgaagcagaggcctggacaaggccttgagtggattggaaatattgatcctagcaatggtggtactaactataatgagaagttcaagagcaaggccacactgactgtagacaaatcctccagcacagcctacatgcagctcagcagcctgacatctgaggactctgcggtctattattgtgcaagttttgcttactggggccaagggactctggtcactgtctctgca

SEQ ID NO: 90 - DNA, 322 bp, Artificial Sequence
  nucleotide sequence of variable light chain 1
gacatccagatgactcagtctccagcctccctatctgcatctgtgggagaaactgtcaccatcacatgtcgagcaagtgggaatattcacaattatttagcatggtatcagcagaaacagggaaaatctcctcagctcctggtctataatgcaaaaaccttagcagatggtgtgccatcaaggttcagtggcagtggatcaggaacacaatattctctcaagatcagcagcctgcggcctgaagattttgggagttattactgtcaacatttttggagtcctccattcacgttcggctcggggacaaagttggaaataaaac

SEQ ID NO: 91 - protein, 112 aa, Artificial Sequence
  Humanized 5E12 IGHV1 69 Heavy Chain
QVQLVQSGAEVKKPGSSVKVSCKASGYTFTNYWMHWVRQAPGQGLEWMGNIDPSNGGTNYNEKFKSRVTITADESTSTAYMELSSLRSEDTAVYYCASFAYWGQGTLVTVSS

SEQ ID NO: 92 - protein, 112 aa, Artificial Sequence
  Humanized 5E12 IGHV1 46 Heavy Chain
QVQLVQSGAEVKKPGASVKVSCKASGYTFTNYWMHWVRQAPGQGLEWMGNIDPSNGGTNYNEKFKSRVTMTRDTSTSTVYMELSSLRSEDTAVYYCASFAYWGQGTLVTVSS

SEQ ID NO: 93 - protein, 112 aa, Artificial Sequence
  Mouse 5E12 scFv PSE2 Heavy chain
QVQLQHPGTEVVKPGASVKLSCKASGYTFTNYWMHWVKQRPGQGLEWIGNIDPSNGGTNYNEKFKSKATLTVDKSSSTAYMQLSSLTSEDSAVYYCASFAYWGQGTLVTVSS

SEQ ID NO: 94 - protein, 112 aa, Artificial Sequence
  consenseus sequence  of VH region of humanized and mouse antibodies
QVQLXXXGXEVXKPGASVKXSCKASGYTFTNYWMHWVRQXPGQGLEWXGNIDPSNGGTNYNEKFKSXXTXTXDXSXSTXYMXLSSLXSEDXAVYYCASFAYWGQGTLVTVSS

SEQ ID NO: 95 - protein, 112 aa, Artificial Sequence
  consensus sequence of VH regions of humanized antibodies
QVQLVQSGAEVKKPGASVKVSCKASGYTFTNYWMHWVRQAPGQGLEWMGNIDPSNGGTNYNEKFKSRVTXTXDXSTSTXYMELSSLRSEDTAVYYCASFAYWGQGTLVTVSS

SEQ ID NO: 96 - protein, 107 aa, Artificial Sequence
  Humanized 5E12 DPK9 Light Chain
DIQMTQSPSSLSASVGDRVTITCRASGNIHNYLAWYQQKPGKAPKLLIYNAKTLADGVPSRFSGSGSGTDFTLTISSLQPEDFATYYCQHFWSPPFTFGQGTKVEIK

SEQ ID NO: 97 - protein, 107 aa, Artificial Sequence; identical to SEQ ID NO: 87
  Mouse 5E12 scFv PSE2 Light Chain

SEQ ID NO: 98 - protein, 107 aa, Artificial Sequence
  consensus sequence of VL chain of humanized and mouse 5E12 antibodies
DIQMTQSPXSLSASVGXXVTITCRASGNIHNYLAWYQQKXGKXPXLLXYNAKTLADGVPSRFSGSGSGTXXXLXISSLXPEDFXXYYCQHFWSPPFTFGXGTKXEIK

SEQ ID NO: 99 - protein, 119 aa, Artificial Sequence
  scFv-3 heavy chain
EVQLLESGGGLVQPGGSLRLSCAASGFRFSDEDMGWVRQAPGKGLEWVSSIYGPSGSTYYADSVKGRFTISRDNSKNTLYLQMNSLRAEDTAVYYCAKDDVGTGTFDYWGQGTLVTVSS

SEQ ID NO: 100 - protein, 121 aa, Artificial Sequence
  scFv-7 heavy chain
EVQLLESGGGLVQPGGSLRLSCAASGFRFDYEDMGWVRQAPGKGLEWVSAIASDGDSTYYADSVKGRFTISRDNSKNTLYLQMNSLRAEDTAVYYCAKIYYDGAYRVFDYWGQGTLVTVSS

SEQ ID NO: 101 - protein, 119 aa, Artificial Sequence
  scFv-6 heavy chain
EVQLLESGGGLVQPGGSLRLSCAASGFRFSAEDMGWVRQAPGKGLEWVSSIASAGASTYYADSVKGRFTISRDNSKNTLYLQMNSLRAEDTAVYYCAKHMYSYAIFDYWGQGTLVTVSS

SEQ ID NO: 102 - protein, 121 aa, Artificial Sequence
  consensus sequence of VH of human antibodies
EVQLLESGGGLVQPGGSLRLSCAASGFRFXXEDMGWVRQAPGKGLEWVSXIXXXXXSTYYADSVKGRFTISRDNSKNTLYLQMNSLRAEDTAVYYCAKXXXXXXXXXFDYWGQGTLVTVSS

SEQ ID NO: 103 - protein, 108 aa, Artificial Sequence
  scFv-3 light chain
DIQMTQSPSSLSASVGDRVTITCRASQYIYYALNWYQQKPGKAPKLLIYDYSALQSGVPSRFSGSGSGTDFTLTISSLQPEDFATYYCQQSYYHPATFGQGTKVEIKR

SEQ ID NO: 104 - protein, 108 aa, Artificial Sequence
  scFv-7 light chain
DIQMTQSPSSLSASVGDRVTITCRASQSISSYLNWYQQKPGKAPKLLIYSASYLQSGVPSRFSGSGSGTDFTLTISSLQPEDFATYYCQQYSMLPTTFGQGTKVEIKR

SEQ ID NO: 105 - protein, 108 aa, Artificial Sequence
  scFv-6 light chain
DIQMTQSPSSLSASVGDRVTITCRASQDIADALNWYQQKPGKAPKLLIYAASSLQSGVPSKFSGSGSGTDFTLTISSLQPEDFATYYCQQGYVHPPTFGQGTKVEIKR

SEQ ID NO: 106 - protein, 108 aa, Artificial Sequence
  consensus sequence of VL of human antibodies
DIQMTQSPSSLSASVGDRVTITCRASQXIXXXLNWYQQKPGKAPKLLIYXXSXLQSGVPSKFSGSGSGTDFTLTISSLQPEDFATYYCQQXXXXPXTFGQGTKVEIKR

SEQ ID NO: 107 - protein, 17 aa, Artificial Sequence
  biotin-NPY20-36
YYSALRHYINLITRQRY

SEQ ID NO: 108 - protein, 36 aa, Artificial Sequence; identical to SEQ ID NO: 1
  biotin-NPY

SEQ ID NO: 109 - protein, 36 aa, Artificial Sequence; identical to SEQ ID NO: 3
  biotin-hPYY

SEQ ID NO: 110 - protein, 36 aa, Artificial Sequence; identical to SEQ ID NO: 5
  biotin-hPP

SEQ ID NO: 111 - DNA, 729 bp, Artificial Sequence
  scFv-3 DNA sequence
gaggtgcagctgttggagtctgggggaggcttggtacagcctggggggtccctgcgtctctcctgtgcagcctccggatttaggttttctgatgaggatatgggctgggtccgccaggctccagggaagggtctagagtgggtatcaagcatttatggccctagcggtagcacatactacgcagactccgtgaagggccggttcaccatctcccgtgacaattccaagaacacgctgtatctgcaaatgaacagcctgcgtgccgaggacaccgcggtatattattgcgcgaaagacgacgttggtactggtacttttgactactggggtcagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagtatatttattatgctttaaattggtatcagcagaaaccagggaaagcccctaagctcctgatctatgattattccgctttgcaaagtggggtcccatcaaggttcagtggcagtggatctgggacagatttcactctcaccatcagcagtctgcaacctgaagattttgcaacttactactgtcaacagtcttactaccatcctgctacgttcggccaagggaccaaggtggaaatcaaacgg

SEQ ID NO: 112 - protein, 243 aa, Artificial Sequence
  scFv-3 amino acid sequence
EVQLLESGGGLVQPGGSLRLSCAASGFRFSDEDMGWVRQAPGKGLEWVSSIYGPSGSTYYADSVKGRFTISRDNSKNTLYLQMNSLRAEDTAVYYCAKDDVGTGTFDYWGQGTLVTVSSGGGGSGGGGSGGGGSTDIQMTQSPSSLSASVGDRVTITCRASQYIYYALNWYQQKPGKAPKLLIYDYSALQSGVPSRFSGSGSGTDFTLTISSLQPEDFATYYCQQSYYHPATFGQGTKVEIKR

SEQ ID NO: 113 - DNA, 729 bp, Artificial Sequence
  scFv-6 DNA sequence
gaggtgcagctgttggagtctgggggaggcttggtacagcctggggggtccctgcgtctctcctgtgcagcctccggatttaggttttctgctgaggatatgggctgggtccgccaggctccagggaagggtctagagtgggtatcatctattgctagtgctggtgctagcacatactacgcagactccgtgaagggccggttcaccatctcccgtgacaattccaagaacacgctgtatctgcaaatgaacagcctgcgtgccgaggacaccgcggtatattattgcgcgaaacatatgtactcttacgctatctttgactactggggtcagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcaggatattgctgatgctttaaattggtatcagcagaaaccagggaaagcccctaagctcctgatctatgctgcatccagtttgcaaagtggggtcccatcaaagttcagtggcagtggatctgggacagatttcactctcaccatcagcagtctgcaacctgaagattttgcaacttactactgtcaacagggttacgttcatcctccgacgttcggccaagggaccaaggtggaaatcaaacgg

SEQ ID NO: 114 - protein, 243 aa, Artificial Sequence
  scFv-6 amino acid sequence
EVQLLESGGGLVQPGGSLRLSCAASGFRFSAEDMGWVRQAPGKGLEWVSSIASAGASTYYADSVKGRFTISRDNSKNTLYLQMNSLRAEDTAVYYCAKHMYSYAIFDYWGQGTLVTVSSGGGGSGGGGSGGGGSTDIQMTQSPSSLSASVGDRVTITCRASQDIADALNWYQQKPGKAPKLLIYAASSLQSGVPSKFSGSGSGTDFTLTISSLQPEDFATYYCQQGYVHPPTFGQGTKVEIKR

SEQ ID NO: 115 - DNA, 735 bp, Artificial Sequence
  scFv-7 DNA sequence
gaggtgcagctgttggagtctgggggaggcttggtacagcctggggggtccctgcgtctctcctgtgcagcctccggatttaggtttgattatgaggatatgggctgggtccgccaggctccagggaagggtctagagtgggtatcagctattgctagtgatggtgatagcacatactacgcagactccgtgaagggccggttcaccatctcccgtgacaattccaagaacacgctgtatctgcaaatgaacagcctgcgtgccgaggacaccgcggtatattattgcgcgaaaatctactacgacggtgcttaccgtgtctttgactactggggtcagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatagtgcatcctatttgcaaagtggggtcccatcaaggttcagtggcagtggatctgggacagatttcactctcaccatcagcagtctgcaacctgaagattttgcaacttactactgtcaacagtactctatgctgcctactacgttcggccaagggaccaaggtggaaatcaaacgg

SEQ ID NO: 116 - protein, 245 aa, Artificial Sequence
  scFv-7 amino acid sequence
EVQLLESGGGLVQPGGSLRLSCAASGFRFDYEDMGWVRQAPGKGLEWVSAIASDGDSTYYADSVKGRFTISRDNSKNTLYLQMNSLRAEDTAVYYCAKIYYDGAYRVFDYWGQGTLVTVSSGGGGSGGGGSGGGGSTDIQMTQSPSSLSASVGDRVTITCRASQSISSYLNWYQQKPGKAPKLLIYSASYLQSGVPSRFSGSGSGTDFTLTISSLQPEDFATYYCQQYSMLPTTFGQGTKVEIKR

SEQ ID NO: 117 - DNA, 708 bp, Artificial Sequence
  h5E12 IGHV1-46/DPK9 scFv DNA sequence
caggtgcagctggtgcagtctggggctgaggtgaagaagcctggggcctcagtgaaggtttcctgcaaggcatctggctacaccttcaccaactactggatgcactgggtgcgacaggcccctggacaagggcttgagtggatgggaaatattgatcctagcaatggtggtactaactataatgagaagttcaagagcagagtcaccatgaccagggacacgtccacgagcacagtctacatggagctgagcagcctgagatctgaggacacggccgtgtattactgtgcaagttttgcttactggggccagggaaccctggtcaccgtctcgagcggtggaggcggttcaggcggaggtggcagcggcggtggcgggtctacggacatccagatgacccagtctccatcctccctgtctgcatctgtaggagacagagtcaccatcacttgccgggcaagtgggaatattcacaattatttagcatggtatcagcagaaaccagggaaagcccctaagctcctgatctataatgcaaaaaccttagcagatggggtcccatcaaggttcagtggcagtggatctgggacagatttcactctcaccatcagcagtctgcaacctgaagattttgcaacttactactgtcaacatttttggagtcctccattcacgttcggccaagggaccaaggtggaaatcaaacgg

SEQ ID NO: 118 - protein, 236 aa, Artificial Sequence
  h5E12 IGHV1-46/DPK9 scFv amino acid sequence
QVQLVQSGAEVKKPGASVKVSCKASGYTFTNYWMHWVRQAPGQGLEWMGNIDPSNGGTNYNEKFKSRVTMTRDTSTSTVYMELSSLRSEDTAVYYCASFAYWGQGTLVTVSSGGGGSGGGGSGGGGSTDIQMTQSPSSLSASVGDRVTITCRASGNIHNYLAWYQQKPGKAPKLLIYNAKTLADGVPSRFSGSGSGTDFTLTISSLQPEDFATYYCQHFWSPPFTFGQGTKVEIKR

SEQ ID NO: 119 - DNA, 708 bp, Artificial Sequence
  h5E12 IGHV1-69/DPK9 scFv DNA sequence
caggtgcagctggtgcagtctggggctgaggtgaagaagcctgggtcctcggtgaaggtctcctgcaaggcttctggctacaccttcaccaactactggatgcactgggtgcgacaggcccctggacaagggcttgagtggatgggaaatattgatcctagcaatggtggtactaactataatgagaagttcaagagcagagtcacgattaccgcggacgaatccacgagcacagcctacatggagctgagcagcctgagatctgaggacacggccgtgtattactgtgcaagttttgcttactggggccagggaaccctggtcaccgtctcgagcggtggaggcggttcaggcggaggtggcagcggcggtggcgggtctacggacatccagatgacccagtctccatcctccctgtctgcatctgtaggagacagagtcaccatcacttgccgggcaagtgggaatattcacaattatttagcatggtatcagcagaagccagggaaagcccctaagctcctgatctataatgcaaaaaccttagcagatggggtcccatcaaggttcagtggcagtggatctgggacagatttcactctcaccatcagcagtctgcaacctgaagattttgcaacttactactgtcaacatttttggagtcctccattcacgttcggccaagggaccaaggtggaaatcaaacgg

SEQ ID NO: 120 - protein, 236 aa, Artificial Sequence
  h5E12 IGHV1-69/DPK9 scFv amino acid sequence
QVQLVQSGAEVKKPGSSVKVSCKASGYTFTNYWMHWVRQAPGQGLEWMGNIDPSNGGTNYNEKFKSRVTITADESTSTAYMELSSLRSEDTAVYYCASFAYWGQGTLVTVSSGGGGSGGGGSGGGGSTDIQMTQSPSSLSASVGDRVTITCRASGNIHNYLAWYQQKPGKAPKLLIYNAKTLADGVPSRFSGSGSGTDFTLTISSLQPEDFATYYCQHFWSPPFTFGQGTKVEIKR

(19) United States
(12) Patent Application Publication
(10) Pub. No.: US 2015/0307599 A1
(43) Pub. Date: Oct. 29, 2015

(54) ANTI-NPY AND PYY ANTIBODIES AND USES THEREOF

(71) Applicant: Garvan Institute of Medical Research, Darlinghurst, New South Wales (AU)

(72) Inventors: Martijn Bijker, Marsfield (AU); Herbert Herzog, Darlinghurst (AU); Fabienne McKay, Prahran (AU); Romain Rouet, Darlinghurst (AU); Peter Schofield, Darlinghurst (AU); David Zahra, Darlinghurst (AU); Daniel Christ, Darlinghurst (AU)

(21) Appl. No.: 14/362,112

(22) PCT Filed: Dec. 3, 2012
(86) PCT No.: PCT/AU2012/001464

§ 371 Date: May 30, 2014

Related U.S. Application Data
(60) Provisional application No. 61/566,247, filed on Dec. 2, 2011.

(51) Int. Cl. C07K 16/18; A61K 45/06; A61K 39/395

(52) U.S. Cl. CPC C07K 16/18; A61K 39/3955; A61K 45/06; C07K 2317/92; C07K 2317/30; C07K 2317/33; C07K 2317/76; C07K 2317/56; C07K 2317/31; C07K 2317/565; C07K 2317/24; A61K 2039/505

(57) ABSTRACT

The present disclosure provides proteins comprising antibody variable regions that bind specifically to NPY and PYY and uses thereof and methods of treating or preventing cancer or inducing an immune response by inhibiting NPY and PYY.

### RELATED APPLICATION DATA

The present application claims priority from U.S. Patent Application No. 61/566,247 entitled “ANTI-NPY AND PYY ANTIBODIES AND USES THEREOF”, filed on 2 Dec. 2012.

### FIELD OF THE INVENTION

The present disclosure relates to reagents and methods for treating cancer and weight loss.

### SEQUENCE LISTING

The present application is filed with a Sequence Listing in electronic form. The entire contents of the Sequence Listing are hereby incorporated by reference.

### BACKGROUND OF THE INVENTION

According to the World Health Organization (WHO), cancer is a leading cause of human death. Worldwide, cancer accounted for at least 7.6 million deaths in 2008, which was about 13% of all deaths that year. The most common forms of cancer are lung cancer (1.3 million deaths in 2008), stomach cancer (803,000 deaths in 2008), colorectal cancer (639,000 deaths in 2008), liver cancer (610,000 deaths in 2008), and breast cancer (519,000 deaths in 2008) Deaths from cancer continue to rise each year and WHO predicts in excess of 11 million cancer caused deaths by 2030. The National Cancer Institute estimates that the cost of cancer care in USA in 2010 alone was US$124.5 billion dollars.

Generally, cancer is caused by uncontrolled proliferation of cells in tissue of a subject. These cells can invade nearby tissues and, ultimately, can spread to more distant part of the body.

Current commonly used therapies for cancer include radiation therapy and chemotherapy drugs. Both of these strategies are toxic. For example, chemotherapy drugs generally kill numerous cells types in a subject in addition to cancer cells, often making the subject unhealthy.

Given the high incidence of cancer and cost to society, new cancer therapies are desirable. Exemplary cancer therapies will have reduced toxicity, e.g., compared to some forms of chemotherapy and/or radiation therapy.

### SUMMARY OF THE INVENTION

The present disclosure is based, in part, on the inventors' determination that inhibiting neuropeptide Y (NPY) and peptide YY (PYY) signaling in a subject prevents development of cancer and can be used to treat cancer. For example, using mice that do not express NPY and PYY, the inventors have shown that mice injected with melanoma cells, lung cancer cells or breast cancer cells either do not develop cancer or are resistant to cancer progression and have a longer survival time than mice expressing NPY and PYY. The inventors also showed that this anti-cancer effect did not occur in mice lacking expression of either NPY or PYY, i.e., inhibition of both proteins was required. Based on these findings, the inventors produced antibodies that bound to and inhibited both NPY and PYY. An anti-NPY and PYY antibody produced by the inventors was shown to reduce tumor size and to significantly increase survival in mice injected with lung cancer cells or breast cancer cells. Thus, the inventors showed that their methods of treating or preventing cancer were generally applicable.

Without being bound by theory of mode of action, the inventors also showed that an anti-cancer effect provided by inhibiting NPY and PYY signaling could be suppressed in at least some models by depleting T cells in the model. These data indicate that one mechanism by which inhibiting NPY and PYY signaling provides an anti-cancer effect is by enhancing an immune response, e.g., an anti-cancer immune response in a subject. These findings suggest the general applicability of the methods produced by the inventors in the treatment or prevention of a variety of cancers. These findings also indicate that the methods may provide a dual mechanism of treating some cancers, e.g., those dependent on NPY/PYY signaling for survival/growth.

Administration of an anti-NPY and PYY antibody produced by the inventors also significantly increased body weight in a mouse to which it was administered. These results indicate that anti-NPY and PYY antibodies (or proteins comprising one or more variable regions thereof) are useful for treating conditions such as anorexia and/or wasting conditions (e.g., cachexia).

The inventors also produced a variety of compounds capable of binding to NPY and PYY, including mouse monoclonal antibodies, humanized antibodies and human antibodies and proteins comprising the binding domains thereof.

These findings by the inventors provide reagents and methods for the treatment of cancer.

The present disclosure provides an isolated or recombinant NPY and PYY-binding protein comprising an antibody variable region, wherein the protein specifically binds to NPY and PYY. In this regard, the skilled person will understand that the protein binds to NPY and PYY by virtue of the antibody variable region.

In one example, the NPY and PYY-binding protein binds to NPY and/or PYY with an affinity at least 100 times greater than it binds to pancreatic polypeptide (PP).

For example, the protein binds to NPY and/or PYY with an affinity at least 100 times greater than it binds to PP.

For example, the protein binds to NPY and/or PYY with a K_{D} of 1×10⁻⁸M or less, such as a K_{D} of 9×10⁻⁹M or less, for example a K_{D} of 8×10⁻⁹M or less, for example, a K_{D} of 7×10⁻⁹M or less. In one example, the protein binds to NPY and/or PYY with a K_{D} of about 5×10⁻⁹M or less. In one example, the protein binds to NPY with a K_{D} of 9×10⁻⁹M or less, such as 5×10⁻⁹M or less. In one example, the protein binds to PYY with a K_{D} of about 4×10⁻⁹M or less. The protein also binds to PP with a K_{D} of about 5×10⁻⁷M or more, for example, a K_{D} of about 7×10⁻⁷M or more, such as a K_{D} of about 9×10⁻⁷M or more. For example, the protein binds to PP with a K_{D} of about 1×10⁻⁶M or more. In one example, the K_{D} is determined using a biosensor, e.g., Biacore or Octet.

In one example, the protein binds to NPY and/or PYY with a K_{D} of 9×10⁻⁴M or less as determined using a biosensor, e.g., Biacore, in which biotinylated human PYY or NPY is immobilized and contacted with a scFv comprising variable regions of the protein. For example, the K_{D} is 9×10⁻⁶M or less, for example, 1×10⁻⁶M or less, for example, 9×10⁻⁷ or less, such as 1×10⁻⁷ or less, for example, 4×10⁻⁸ or less.

In one example, the protein binds to NPY with a K_{D} of 9×10⁻⁶M or less as determined using a biosensor, e.g., Biacore, in which biotinylated human NPY is immobilized and contacted with a scFv comprising variable regions of the protein. For example, the K_{D} is 9×10⁻⁶M or less, for example, 1×10⁻⁶M or less, for example, 9×10⁻⁷ or less, such as 1×10⁻⁷ or less, for example, 4×10⁻⁸ or less.

In one example, the protein binds to PYY with a K_{D} of 4×10⁻⁵M or less as determined using a biosensor, e.g., Biacore, in which biotinylated human PYY is immobilized and contacted with a scFv comprising variable regions of the protein. For example, the K_{D} is 9×10⁻⁶M or less, for example, 3×10⁻⁶M or less, for example, 1×10⁻⁶ or less, such as 4×10⁻⁸ or less.

For example, the protein dissociates from NPY and/or PYY with a dissociation rate constant (k_{d}) at least 100 times slower than it dissociates from PP. For example, the protein binds to NPY and/or PYY with a k_{d} of 3×10⁻¹s⁻¹ or less, for example, 2×10⁻²s⁻¹ or less, for example, 5×10⁻³s⁻¹ or less, for example 1×10⁻³s⁻¹ or less, such as a k_{d} of 9×10⁻⁴ s⁻¹ or less, for example a k_{d} of 8×10⁻⁴ s⁻¹ or less, for example, a k_{d} of 7×10⁻⁴ s⁻¹ or less. In one example, the protein binds to NPY and/or PYY with a k_{d} of about 6×10⁻⁴ s⁻¹ or less. In one example, the protein binds to NPY with a k_{d} of about 1.5×10⁻³s⁻¹ or less or 4.5×10⁻⁴ s⁻¹ or less. In one example, the protein binds to PYY with a k_{d} of about 6×10⁻⁴ s⁻¹ or less. The protein also binds to PP with a k_{d} of about 5×10⁻³ s⁻¹ or more, for example, a k_{d} of about 7×10⁻³M or more, such as a k_{d} of about 9×10⁻³M or more. For example, the protein binds to PP with a k_{d} of about 1×10⁻²M or more. For example, the protein binds to PP with a k_{d} of about 5×10⁻²M or more. In one example, the k_{d} is determined using a biosensor, e.g., Biacore or Octet.

In one example, the protein dissociates from NPY and/or PYY with a k_{d} of 3.3×10⁻¹s⁻¹ or less as determined using a biosensor, e.g., Biacore, in which biotinylated human NPY or PYY is immobilized and contacted with a scFv comprising variable regions of the protein. In one example, the K_{d} is 2×10⁻²s⁻¹ or less, such as, 1.2×10⁻¹s⁻¹ or less, for example, 8×10⁻³s⁻¹ or less, for example, 6×10⁻³s⁻¹ or less, such as, 4×10⁻³s⁻¹ or less.

In one example, the protein binds to human NPY and human PYY.

In one example, the protein binds to mouse NPY and mouse PYY.

In one example, the protein binds to human and mouse NPY and human and mouse PYY.

In one example, the NPY and PYY-binding protein does not significantly or detectably bind to PP. For example, the binding to PP is not detectable when assessed using an enzyme linked immunosorbent assay (ELISA). For example, the binding to PP is not detectable when assessed using a biosensor, e.g., Biacore, in which biotinylated human PP is immobilized and contacted with the protein or a scFv comprising variable regions of the protein. By not significantly or detectably binding to PP, such an antibody can avoid unwanted side effects, such as, effects on pancreatic secretion and/or excessive glucose tolerance and/or cardiovascular effects, such as increased heart rate.

In one example, the NPY and PYY-binding protein binds to an epitope comprising in amino-carboxy order:
(i) a positively charged amino acid, a positively charged amino acid and an amidated tyrosine; or
(ii) an amino acid structurally related to glutamine, a positively charged amino acid and an amidated tyrosine; or
(iii) XRY-amide, wherein X is Q or K or R (SEQ ID NO: 79).

Exemplary positively charged amino acids are arginine and lysine.

Exemplary amino acids structurally related to glutamine are asparagine, glutamic acid and glutamine.

In one example, the NPY and PYY-binding protein binds to an epitope comprising the sequence QRY-amide (SEQ ID NO: 80). In one example, the tyrosine residue is positioned at the C-terminus of the peptide. In one example, the protein binds to a peptide comprising or consisting of a sequence set forth in any one of SEQ ID NOs: 7-21, 38-52, 56, 58, 61, 62, 78 or 79. In another example, the NPY and PYY-binding protein does not detectably bind to an epitope comprising or consisting of the sequence PQRY-amide (SEQ ID NO: 67).

The present disclosure also provides an NPY and PYY-binding protein comprising an antibody variable region, wherein the protein specifically binds to NPY and PYY, and wherein the protein binds to an epitope comprising the sequence QRY-amide (SEQ ID NO: 80) at its C terminus. In one example, the epitope comprising the sequence QRY-amide (SEQ ID NO: 80) at its C terminus is contained within a peptide comprising or consisting of a sequence set forth in any one of SEQ ID NOs: 7-21, 38-52, 56, 58, 61, 62, 78 or 79.

In one example, the NPY and PYY-binding protein does not significantly or detectably bind to an epitope comprising the sequence PRY-amide at its C terminus (SEQ ID NO: 81). In one example, the binding to the epitope is determined using a biosensor (e.g., Biacore) or an ELISA, such as an indirect ELISA. In one example, the epitope comprising the sequence PRY-amide (SEQ ID NO: 81) at its C terminus is contained with a peptide comprising or consisting of a sequence set forth in any one of SEQ ID NOs: 5, 6, 59 or 60.

In one example, the NPY and PYY-binding protein binds to an epitope comprising the sequence QRY-amide at its C terminus (SEQ ID NO: 80) with at least 100 times greater affinity than it does to an epitope comprising the sequence PRY-amide at its C terminus (SEQ ID NO: 81). Exemplary K_{D} and K_{d} are described above and are to be taken to apply mutatis mutandis to the present example of the disclosure.

As will be apparent to the skilled artisan from the disclosure herein, the tyrosine residue at the C-terminus of the epitope bound by the NPY and PYY-binding protein comprises a free amide group or is amidated.

In one example, the protein does not significantly or detectably bind to an epitope comprising the sequence QRY at its C terminus (SEQ ID NO: 80), wherein the tyrosine residue at the C-terminus of the epitope lacks a free amide group (or is not amidated) and/or comprises a free carboxy group and/or wherein the tyrosine residue is substituted with a phenylalanine. In one example, the binding to the epitope is determined using an ELISA, such as an indirect ELISA.

In one example, the NPY and PYY-binding protein binds with significantly greater affinity to an epitope comprising a C-terminal tyrosine than it does to an epitope in which the C-terminal tyrosine is substituted with a non-polar amino acid. For example, the NPY and PYY-binding protein does not detectably bind to an epitope in which the C-terminal tyrosine is substituted with a non-polar amino acid.

In one example, the NPY and PYY-binding protein does not significantly or detectably bind to an epitope comprising the sequence FMRF-amide (SEQ ID NO: 82) or FLFQPQRF-amide (SEQ ID NO: 83) or QRF-amide (SEQ ID NO: 84). For example, the NPY and PYY-binding protein does not bind to a peptide comprising the sequence FMRF-amide (SEQ ID NO: 82) or FLFQPQRF-amide (SEQ ID NO: 83) or QRF-amide (SEQ ID NO: 84) at its C-terminus. For example, the protein does not significantly or detectably bind to a peptide comprising or consisting of a sequence set forth in any one of SEQ ID NOs: 63, 64, 65 or 66. In one example, the protein does not significantly or detectably bind to a brain neuropeptide I (e.g., comprising a sequence set forth in SEQ ID NO: 65) or a morphine modulating neuropeptide. In one example, the binding to the epitope is determined using a biosensor (e.g., Biacore) or an ELISA, such as an indirect ELISA. Such specificity is useful, e.g., to reduce off-target effects in a therapeutic context.

In one example, the NPY and PYY-binding protein neutralizes NPY and PYY-mediated signaling in a cell. For example, the NPY and PYY-binding protein neutralizes NPY and/or PYY-mediated ERK phosphorylation.

In one example, the variable domain of the NPY and PYY-binding protein competitively inhibits binding of an antibody comprising a heavy chain variable region (V_{H}) comprising a sequence set forth in SEQ ID NO: 85 or 86 and a light chain variable region (V_{L}) comprising a sequence set forth in SEQ ID NO: 87.

In one example, the variable domain of the NPY and PYY-binding protein competitively inhibits binding of one or more of the following:
(i) an antibody comprising a V_{H} comprising a sequence set forth in SEQ ID NO: 85 or 86 and a V_{L} comprising a sequence set forth in SEQ ID NO: 87;
(ii) an antibody comprising a V_{H} comprising a sequence set forth in SEQ ID NO: 91 and a V_{L} comprising a sequence set forth in SEQ ID NO: 96;
(iii) an antibody comprising a V_{H} comprising a sequence set forth in SEQ ID NO:

92 and a V_{L} comprising a sequence set forth in SEQ ID NO: 96;
(iv) an antibody comprising a V_{H} comprising a sequence set forth in SEQ ID NO: 99 and a V_{L} comprising a sequence set forth in SEQ ID NO: 103;
(v) an antibody comprising a V_{H} comprising a sequence set forth in SEQ ID NO: 100 and a V_{L} comprising a sequence set forth in SEQ ID NO: 104; and/or
(vi) an antibody comprising a V_{H} comprising a sequence set forth in SEQ ID NO: 101 and a V_{L} comprising a sequence set forth in SEQ ID NO: 105.

In one example, the NPY and PYY-binding protein binds to the same epitope as an antibody comprising a V_{H} comprising a sequence set forth in SEQ ID NO: 85 or 86 and a V_{L} comprising a sequence set forth in SEQ ID NO: 87.

In one example, the NPY and PYY-binding protein binds to the same epitope as one or more of the following:
(i) an antibody comprising a V_{H} comprising a sequence set forth in SEQ ID NO: 85 or 86 and a V_{L} comprising a sequence set forth in SEQ ID NO: 87;
(ii) an antibody comprising a V_{H} comprising a sequence set forth in SEQ ID NO: 91 and a V_{L} comprising a sequence set forth in SEQ ID NO: 96;
(iii) an antibody comprising a V_{H} comprising a sequence set forth in SEQ ID NO: 92 and a V_{L} comprising a sequence set forth in SEQ ID NO: 96;
(iv) an antibody comprising a V_{H} comprising a sequence set forth in SEQ ID NO: 99 and a V_{L} comprising a sequence set forth in SEQ ID NO: 103;
(v) an antibody comprising a V_{H} comprising a sequence set forth in SEQ ID NO: 100 and a V_{L} comprising a sequence set forth in SEQ ID NO: 104; and/or
(vi) an antibody comprising a V_{H} comprising a sequence set forth in SEQ ID NO: 101 and a V_{L} comprising a sequence set forth in SEQ ID NO: 105.

The present disclosure additionally or alternatively provides an isolated or recombinant NPY and PYY-binding protein comprising an antibody variable region, wherein (i) the variable region specifically binds to NPY and PYY; (ii) the protein neutralizes NPY and PYY signaling; and (iii) the protein does not significantly and/or detectably bind to any one or more of SEQ ID NOs: 5, 6, 22-37, 53-55, 57, 59, 60, 63-67, 70, 75, 76 or 77.

In one example, the binding to the peptide is determined using a biosensor (e.g., Biacore) or an ELISA, such as an indirect ELISA.

In one example, the NPY and PYY-binding protein comprises a V_{H} and a V_{L}, wherein the V_{H} and V_{L} bind to form a Fv that specifically binds to NPY and PYY.

The present disclosure also provides an isolated or recombinant NPY and PYY-binding protein comprising:

(i) a V_{H} comprising the CDRs of a V_{H} comprising a sequence set forth in SEQ ID NO: 85 or 86; and
(ii) a V_{L} comprising the CDRs of a V_{L} comprising a sequence set forth in SEQ ID NO: 87,
   wherein the V_{H} and V_{L} bind to form a Fv that specifically binds to NPY and PYY.

Exemplary CDRs are shown in FIGS. 6A (V_{H}) and 6B (V_{L}). In one example, the CDRs are defined according to the numbering system of Kabat and comprise the sequences marked in bold in FIG. 6A (V_{H} CDRs) or 6B (V_{L} CDRs). In one example, the CDRs are defined according to the enhanced Chothia numbering system and comprise the underlined sequences in FIG. 6A (V_{H} CDRs) or 6B (V_{L} CDRs).

The present disclosure also provides an isolated or recombinant NPY and PYY-binding protein comprising a V_{H} comprising the CDRs of a V_{H} comprising a sequence set forth in SEQ ID NO: 102 and a V_{L} comprising the CDRs of a V_{L} comprising a sequence set forth in SEQ ID NO: 106, wherein the V_{H} and V_{L} bind to form a Fv that specifically binds to NPY and PYY.

The present disclosure also provides an isolated or recombinant NPY and PYY-binding protein comprising:
(i) a V_{H} comprising the CDRs of a V_{H} comprising a sequence set forth in SEQ ID NO: 99 and a V_{L} comprising the CDRs of a V_{L} comprising a sequence set forth in SEQ ID NO: 103;
(ii) a V_{H} comprising the CDRs of a V_{H} comprising a sequence set forth in SEQ ID NO: 100 and a V_{L} comprising the CDRs of a V_{L} comprising a sequence set forth in SEQ ID NO: 104;
(iii) a V_{H} comprising the CDRs of a V_{H} comprising a sequence set forth in SEQ ID NO: 101 and a V_{L} comprising the CDRs of a V_{L} comprising a sequence set forth in SEQ ID NO: 105;
   wherein the V_{H} and V_{L} bind to form a Fv that specifically binds to NPY and PYY.

Exemplary CDRs are shown in FIGS. 19A, 19B and 19D (V_{H}) and 19C and 19E (V_{L}).

In one example, the CDRs are defined according to the numbering system of Kabat and comprise the sequences marked in bold in FIGS. 19A, 19B and 19D (V_{H} CDRs) or 19C and 19E (V_{L} CDRs).

For example, a heavy chain CDR1 comprises amino acids 31-35 of SEQ ID NOs: 85, 86, 91, 92, 93, 94, 95, 99, 100, 101 and 102. A heavy chain CDR2 comprises amino acids 50-66 of SEQ ID NOs: 85, 86, 91, 92, 93, 94, 95, 99, 100, 101 and 102. A heavy chain CDR3 comprises amino acids 99-101 of 85, 86, 91, 92, 93, 94 and 95 or amino acids 99-108 of SEQ ID NOs: 99, 101 and some examples of 102 or amino acids 99-110 of SEQ ID NO: 100 and some examples of 102.

For example, a light chain CDR1 comprises amino acids 24-34 of SEQ ID NOs: 87, 96, 97, 98, 103, 104, 105 and 106. A light chain CDR2 comprises amino acids 50-56 of SEQ ID NOs: 87, 96, 97, 98, 103, 104, 105 and 106. A light chain CDR3 comprises amino acids 89-97 of SEQ ID NOs: 87, 96, 97, 98, 103, 104, 105 and 106.

In one example, the CDRs are defined according to the enhanced Chothia numbering system and comprise the underlined sequences in FIGS. 19A, 19B and 19D (V_{H} CDRs) or 19C and 19E (V_{L} CDRs).

For example, a heavy chain CDR1 comprises amino acids 26-31 of SEQ ID NOs: 85, 86, 91, 92, 93, 94, 95, 99, 100, 101 and 102. A heavy chain CDR2 comprises amino acids 52-57 of SEQ ID NOs: 85, 86, 91, 92, 93, 94, 95, 99, 100, 101 and 102. A heavy chain CDR3 comprises amino acids 99-101 of 85, 86, 91, 92, 93, 94 and 95 or amino acids 99-108 of SEQ ID NOs: 99, 101 and some examples of 102 or amino acids 99-110 of SEQ ID NO: 100 and some examples of 102.

For example, a light chain CDR1 comprises amino acids 24-34 of SEQ ID NOs: 87, 96, 97, 98, 103, 104, 105 and 106. A light chain CDR2 comprises amino acids 50-56 of SEQ ID NOs: 87, 96, 97, 98, 103, 104, 105 and 106. A light chain CDR3 comprises amino acids 89-97 of SEQ ID NOs: 87, 96, 97, 98, 103, 104, 105 and 106.

The present disclosure also provides an isolated or recombinant NPY and PYY-binding protein comprising a V_{H} comprising a sequence set forth in SEQ ID NO: 85 or 86 and/or a V_{L} comprising a sequence set forth in SEQ ID NO: 87 or a chimeric, deimmunized, CDR grafted, humanized or synhumanized form of the V_{H} and/or V_{L}, wherein the V_{H} and V_{L} bind to form a Fv that specifically binds to NPY and PYY.

In one example, the humanized protein comprises a V_{H} comprising a sequence set forth in SEQ ID NO: 94 or 95 and/or a V_{L} comprising a sequence set forth in SEQ ID NO: 96.

In one example, the humanized protein comprises one of the following:
(i) a V_{H} comprising a sequence set forth in SEQ ID NO: 91 and/or a V_{L} comprising a sequence set forth in SEQ ID NO: 96; and/or
(ii) the humanized protein comprises a V_{H} comprising a sequence set forth in SEQ ID NO: 92 and/or a V_{L} comprising a sequence set forth in SEQ ID NO: 96.

The present disclosure also provides an isolated or recombinant NPY and PYY-binding protein comprising a V_{H} comprising a sequence set forth in SEQ ID NO: 102 and/or a V_{L} comprising a sequence set forth in SEQ ID NO: 106.

The present disclosure also provides an isolated or recombinant NPY and PYY-binding protein comprising one of the following:
(i) a V_{H} comprising a sequence set forth in SEQ ID NO: 99 and/or a V_{L} comprising a sequence set forth in SEQ ID NO: 103;
(ii) a V_{H} comprising a sequence set forth in SEQ ID NO: 100 and/or a V_{L} comprising a sequence set forth in SEQ ID NO: 104; or
(iii) a V_{H} comprising a sequence set forth in SEQ ID NO: 101 and/or a V_{L} comprising a sequence set forth in SEQ ID NO: 105.

In one example, the V_{H} and the V_{L} are in a single polypeptide chain. For example, the NPY and PYY-binding protein is:
(i) a single chain Fv fragment (scFv);
(ii) a dimeric scFv (di-scFv); or
(iii) at least one of (i) and/or (ii) linked to a Fc or a heavy chain constant domain (C_{H}) 2 and/or C_{H}3.

In one example, the V_{L} and V_{H} are in separate polypeptide chains. For example, the NPY and PYY-binding protein is:
(i) a diabody;
(ii) a triabody;
(iii) a tetrabody;
(iv) a Fab;
(v) a F(ab′)₂;
(vi) a Fv; or
(iv) one of (i) to (iii) linked to a Fc or a heavy chain constant domain (C_{H}) 2 and/or C_{H}3.

In one example, the NPY and PYY-binding protein is an antibody. In one example, the antibody is chimeric, deimmunized, CDR grafted, humanized or synhumanized.

The present disclosure also provides a NPY and PYY-binding protein, which is an antibody comprising a V_{H} comprising a sequence set forth in SEQ ID NO: 85 or 86 and/or a V_{L} comprising a sequence set forth in SEQ ID NO: 87 or a chimeric, deimmunized, CDR grafted, humanized or synhumanized form of the antibody.

In one example, the antibody comprises a V_{H} comprising a sequence set forth in SEQ ID NO: 85 and a V_{L} comprising a sequence set forth in SEQ ID NO:87 or a chimeric, deimmunized, CDR grafted, humanized or synhumanized form of the V_{H} and V_{L}.

In one example, the antibody comprises a V_{H} comprising a sequence set forth in SEQ ID NO: 86 and a V_{L} comprising a sequence set forth in SEQ ID NO: 87 or a chimeric, deimmunized, CDR grafted, humanized or synhumanized form of the V_{H} and V_{L}.

The present disclosure also provides a NPY and PYY-binding protein, which is an antibody comprising a V_{H} comprising a sequence set forth in SEQ ID NO: 94 or 95 and a V_{L} comprising a sequence set forth in SEQ ID NO: 98.

In one example, the antibody comprises one of the following:
(i) a V_{H} comprising a sequence set forth in SEQ ID NO: 91 and a V_{L} comprising a sequence set forth in SEQ ID NO: 96; and/or
(ii) a V_{H} comprising a sequence set forth in SEQ ID NO: 92 and/or a V_{L} comprising a sequence set forth in SEQ ID NO: 96.

The present disclosure also provides a NPY and PYY-binding protein, which is an antibody comprising a V_{H} comprising a sequence set forth in SEQ ID NO: 102 and/or a V_{L} comprising a sequence set forth in SEQ ID NO: 106.

The present disclosure also provides a NPY and PYY-binding protein, which is an antibody comprising one of the following:
(i) a V_{H} comprising a sequence set forth in SEQ ID NO: 99 and/or a V_{L} comprising a sequence set forth in SEQ ID NO: 103;
(ii) a V_{H} comprising a sequence set forth in SEQ ID NO: 100 and/or a V_{L} comprising a sequence set forth in SEQ ID NO: 104; or
(iii) a V_{H} comprising a sequence set forth in SEQ ID NO: 101 and/or a V_{L} comprising a sequence set forth in SEQ ID NO: 105.

The present disclosure also provides an isolated or recombinant nucleic acid encoding the NPY and PYY-binding protein of the present disclosure. In this regard, the disclosure is not limited to the specific exemplified nucleic acids described herein, but also encompasses any nucleic acid that encodes a NPY and PYY-binding protein of the disclosure as a result of degeneracy of the genetic code. For example, the nucleic acid may be codon optimized for expression in a particular cell type. In one example, a nucleic acid encoding a V_{H} comprises a sequence set forth in SEQ ID NO: 88 or 89. In one example, a nucleic acid encoding a V_{L} comprises a sequence set forth in SEQ ID NO: 90.

In one example, such a nucleic acid is included in an expression construct in which the nucleic acid is operably linked to a promoter. Such an expression construct can be in a vector, e.g., a plasmid.

In examples of the disclosure directed to single polypeptide NPY and PYY-binding proteins, the expression construct may comprise a promoter linked to a nucleic acid encoding that polypeptide chain.

In examples directed to multiple polypeptides that form a NPY and PYY-binding protein, an expression construct of the disclosure comprises a nucleic acid encoding one of the polypeptides (e.g., comprising a V_{H}) operably linked to a promoter and a nucleic acid encoding another of the polypeptides (e.g., comprising a V_{L}) operably linked to a promoter.

In another example, the expression construct is a bicistronic expression construct, e.g., comprising the following operably linked components in 5′ to 3′ order:
(i) a promoter
(ii) a nucleic acid encoding a first polypeptide;
(iii) an internal ribosome entry site; and
(iv) a nucleic acid encoding a second polypeptide.

For example, the first polypeptide comprises a V_{H} and the second polypeptide comprises a V_{L}, or the first polypeptide comprises a V_{L} and the second polypeptide comprises a V_{H}.

The present disclosure also contemplates separate expression constructs one of which encodes a first polypeptide (e.g., comprising a V_{H}) and another of which encodes a second polypeptide (e.g., comprising a V_{L}). For example, the present disclosure also provides a composition comprising:
(i) a first expression construct comprising a nucleic acid encoding a polypeptide (e.g., comprising a V_{H} operably linked to a promoter); and
(ii) a second expression construct comprising a nucleic acid encoding a polypeptide (e.g., comprising a V_{L} operably linked to a promoter),
   wherein the first and second polypeptides associate to form a NPY and PYY-binding protein of the present disclosure.

The present disclosure also provides an isolated cell expressing a NPY and PYY-binding protein of the disclosure or a recombinant cell genetically-modified to express a NPY and PYY-binding protein of the disclosure.

In one example, the cell comprises the expression construct of the disclosure or:
(i) a first expression construct comprising a nucleic acid encoding a polypeptide (e.g., comprising a V_{H}) operably linked to a promoter; and
(ii) a second expression construct comprising a nucleic acid encoding a polypeptide (e.g., comprising a V_{L}) operably linked to a promoter,

wherein the first and second polypeptides associate to form a NPY and PYY-binding protein of the present disclosure.

Examples of cells of the present disclosure include bacterial cells, yeast cells, insect cells or mammalian cells. Exemplary cells are mammalian.

The present disclosure additionally provides methods for producing a NPY and PYY-binding protein of the disclosure. For example, such a method involves maintaining the expression construct(s) of the disclosure under conditions sufficient for the NPY and PYY-binding protein to be produced.

In one example, a method for producing a NPY and PYY-binding protein of the disclosure comprises culturing the cell of the disclosure under conditions sufficient for the protein to be produced and, optionally, secreted.

In one example, the method for producing a NPY and PYY-binding protein of the disclosure additionally comprises isolating the protein.

The present disclosure also provides a composition comprising the NPY and PYY-binding protein, nucleic acid, expression construct or cell of the present disclosure and a suitable carrier. In one example, the composition comprises the NPY and PYY-binding protein of the present disclosure and a suitable carrier. In one example, the carrier is pharmaceutically acceptable, e.g., the composition is a pharmaceutical composition.

The present disclosure additionally provides a method for treating or preventing a NPY and/or PYY-mediated condition, the method comprising administering to a subject the NPY and PYY-binding protein of the disclosure or the composition of the disclosure.

The present disclosure additionally provides a NPY and PYY-binding protein of the disclosure or the composition of the disclosure for use in treating or preventing a NPY and/or PYY-mediated condition.

In one example, the NPY and/or PYY-mediated condition is weight loss, e.g., anorexia or a wasting condition, such as, cachexia, pre-cachexia or sarcopenia (e.g., wasting associated with aging).

The present disclosure additionally provides a method of treating or preventing cancer in a subject, the method comprising inhibiting NPY and PYY signaling in cells of the subject. In this regard, preventing cancer includes preventing metastasis or recurrence of cancer in a subject.

In one example, the method comprises administering to the subject a compound that antagonizes a receptor activated by NPY and/or PYY. For example, the method comprises administering to the subject compound that antagonizes one or more of a Y1 receptor and/or a Y2 receptor and/or a Y4 receptor and/or a Y5 receptor. In one example, the method comprises administering plurality of compounds, e.g., two or more compounds selected from the group consisting of an antagonist of a Y1 receptor, an antagonist of a Y2 receptor, an antagonist of a Y4 receptor and an antagonist of a Y5 receptor. In another example, the method comprises administering two or more proteins selected from the group consisting of a protein comprising an antibody variable region that binds to and inhibits a Y1 receptor, a protein comprising an antibody variable region that binds to and inhibits a Y2 receptor, a protein comprising an antibody variable region that binds to and inhibits a Y4 receptor and a protein comprising an antibody variable region that binds to and inhibits a Y5 receptor. In another example, the method comprises administering a protein comprising a plurality of variable regions, the protein being capable of binding to and inhibiting two or more receptors selected from the group consisting of a Y1 receptor, a Y2 receptor, a Y4 receptor and a Y5 receptor.

In one example, the method comprises administering to the subject one or more compounds that bind to NPY and/or PYY to thereby inhibit NPY and PYY signaling in cells of the subject.

In one example, the compound does not detectably or significantly bind to or inhibit PP.

In one example, the compound(s) is(are) a protein(s) comprising an antibody variable region.

In one exemplary form of the disclosure, the method comprises administering a protein comprising an antibody variable region that binds to and inhibits PYY and a protein comprising an antibody variable region that binds to and inhibits NPY.

For example, the method comprises administering a single protein that binds to and inhibits NPY and PYY.

In one example, the method comprises administering a protein comprising an antibody variable region that binds to and inhibits PYY and an antibody variable region that binds to and inhibits NPY. For example, the protein is a bispecific antibody or a bispecific diabody, triabody or tetrabody.

In one example, the method comprises administering a protein comprising an antibody variable region that binds to NPY and PYY.

In one example, the method comprises administering the NPY and PYY-binding protein of the disclosure or the composition of the disclosure.

In one example, the cancer is not neuroblastoma.

In another example, the cancer is selected from the group consisting of an adenocarcinoma, a squamous cell carcinoma, a digestive/gastrointestinal cancer, an eye cancer, a musculoskeletal cancer, a breast cancer, a genitourinary cancer, a germ cell cancer, a head and neck cancer, a hematologic/blood cancer, a respiratory cancer, a skin cancer, an AIDS-related malignancy or a genealogic cancer. For example, the cancer is lung cancer or melanoma or breast cancer.

In one example, the cancer expresses a NPY receptor responsive to NPY and PYY. For example, the cancer expresses a Y1 receptor and/or a Y2 receptor and/or a Y4 receptor and/or a Y5 receptor. In accordance with this example, the method of the disclosure can comprise detecting expression of the Y receptor(s).

In one example, the cancer does not express a NPY receptor responsive to NPY and PYY.

In one example, the cancer does not proliferate in response to treatment with NPY and/or PYY.

In one example, the method of the disclosure comprises administering an amount of the compound sufficient to induce or enhance an immune response against the cancer in the subject. For example, the immune response is a T cell response.

The present disclosure also contemplates administering a further compound to treat the cancer (e.g., chemotherapy) or exposing the subject to radiation therapy.

In one example, the subject additionally suffers from a wasting condition, e.g., cancer cachexia, and the method additionally treats the wasting condition. Thus, methods described herein for treating cancer shall be taken to apply mutatis mutandis to treatment of cancer accompanied by a wasting condition, such as cachexia.

In one example, the method of the present disclosure additionally comprises administering a further compound to treat the cancer or exposing the subject to radiation therapy.

In one example, the disclosure provides a method for treating cancer, the method comprises performing a method as described herein comprising inhibiting NPY and PYY and following treatment (e.g., when the cancer in the subject is smaller or is not progressing or when the subject's body weight has increased or stabilized) administering a further compound to treat the cancer or exposing the subject to radiation therapy.

The present disclosure additionally provides (an) inhibitor(s) of NPY and PYY signaling for the treatment or prevention of cancer in a subject.

The present disclosure also provides a method for inducing or enhancing an immune response in a subject, the method comprising inhibiting NPY and PYY signaling in cells of the subject.

In one example, the subject suffers from cancer and the immune response is against the cancer or a cell thereof.

For example, the method comprises administering to the subject one or more compounds that bind to NPY and/or PYY to thereby inhibit NPY and PYY signaling in cells of the subject.

For example, the method comprises administering a NPY and PYY binding protein of the disclosure or a composition comprising same. Other suitable compounds and combinations thereof are described herein and are to be taken to apply mutatis mutandis to the present example of the disclosure.

The present disclosure also provides an inhibitor(s) of NPY and PYY for inducing or enhancing an immune response against a cancer cell.

The present disclosure also provides a method of inducing or enhancing an immune response in a subject, the method comprising administering the NPY and PYY binding-protein of the disclosure or a composition comprising same.

The present disclosure also provides the NPY and PYY binding-protein of the disclosure or a composition comprising same for inducing or enhancing an immune response in a subject.

### BRIEF DESCRIPTION OF THE DRAWINGS

FIG. 1A is a graphical representation showing percent survival of wild-type mice (wt) and mice deficient in NPY and PYY (NPY^{-/-}PYY^{-/-}) following administration of B16F10 melanoma cells.

FIG. 1B is a graphical representation showing percent survival of wild-type mice (wt), mice deficient in NPY (NPY^{-/-}), mice deficient in PYY (PYY^{-/-}) and mice deficient in NPY and PYY (NPY^{-/-}PYY^{-/-}) following administration of LL2 Lewis lung carcinoma cells.

FIG. 2A is a graphical representation showing the mean size (+/−SEM) of B16F10 melanoma tumors in wild-type mice (wt) or in mice deficient in NPY and PYY (NPY^{-/-}PYY^{-/-}) at the indicated days.

FIG. 2B is a graphical representation showing the mean size (+/−SEM) of LL2 Lewis lung carcinoma tumors in wild-type mice (wt), mice deficient in NPY (NPY^{-/-}), mice deficient in PYY (PYY^{-/-}) and mice deficient in NPY and PYY (NPY^{-/-}PYY^{-/-}) at the indicated days.

FIG. 3 is a copy of a photographic representation of B16F10 melanoma tumors recovered from wild-type mice (wt) and mice deficient in NPY and PYY (NPY^{-/-}PYY^{-/-}.)

FIG. 4 is a graphical representation showing the mean size (+/−SEM) of B16F10 melanoma tumors from mice deficient in NPY and PYY (NPY^{-/-}PYY^{-/-}) and NPY^{-/-} PYY^{-/-} mice in which T cells have been depleted, at the indicated days.

FIG. 5 is a graphical representation showing an alignment of human and mouse NPY and PYY. The underlined sequence was used to immunize mice to generate anti-NPY/PYY antibodies.

FIG. 6A is a graphical representation showing sequences of heavy chain variable regions of antibodies. Boxed regions contain CDRs (as indicated) as defined by the Kabat numbering system and the enhanced Chothia numbering system. CDRs defined by the Kabat numbering system are shown in bold. CDRs defined by the enhanced Chothia numbering system are underlined.

FIG. 6B is a graphical representation showing sequences of light chain variable regions of antibodies. Boxed regions contain CDRs (as indicated) as defined by the Kabat numbering system and the enhanced Chothia numbering system. CDRs defined by the Kabat numbering system are shown in bold. CDRs defined by the enhanced Chothia numbering system are underlined.

FIG. 7 is a graphical representation showing the level of binding of MAb 5E12-B7 (used interchangeably herein with “5E12) to the recited peptides as determined using ELISA.

FIG. 8 is a graphical representation showing the level of binding of MAb 5E12-B7 to the recited peptides as determined using ELISA.

FIG. 9 is a graphical representation showing the level of binding of MAb 5E12-B7 to the recited peptides as determined using Biacore.

FIG. 10 is a graphical representation showing the level of binding of MAb 5E12-B7 to the recited peptides as determined using Biacore.

FIG. 11A is a graphical representation showing the level of binding of MAb 5E12-B7 to the recited peptides as determined using Biacore.

FIG. 11B is a graphical representation showing the level of binding of MAb 5E12-B7 to the recited peptides as determined using Biacore.

FIG. 12 is a graphical representation showing the level of binding of MAb 5E12-B7 to the recited peptides as determined using Biacore.

FIG. 13 is a copy of a series of photographic representations showing the time (0-20 min) dependent rise and fall of phosphorylated ERK in cells treated with NPY or NPY and MAb 5E12-B7. Total ERK is shown below.

FIG. 14A is a graphical representation showing the mean size (+/−SEM) of LL2 Lewis lung carcinoma tumors recovered from wild-type mice (wt) treated with either isotype control antibody or MAb 5E12-B7. Treatment with isotype control antibody or MAb 5E12-B7 was performed on the days indicated by the arrows.

FIG. 14B is a graphical representation showing percentage survival of mice administered LL2 Lewis lung carcinoma tumor cells and treated with either isotype control antibody or MAb 5E12-B7.

FIG. 15 comprises a series of graphical representations showing proliferation of T cells from mice treated with isotype control antibody (cIg) or MAb 5E12-B7 and activated with anti-CD3 antibody. CD4 and CD8 subsets of T cells were studied as indicated.

FIG. 16A is a graphical representation showing absolute body weights of mice treated with MAb 5E12-B7 or saline. Data are the means of 8 female WT mice in each group. 2-way ANOVA for statistics, with p value indicated.

FIG. 16B is a graphical representation showing body weights of mice treated with MAb 5E12-B7 or saline expressed as percent of initial body weight. Data are the means of 8 female WT mice in each group. 2-way ANOVA for statistics, with p value indicated.

FIG. 17A is a graphical representation showing weights of white adipose tissue (WAT) from 4 depots of mice treated for eight weeks with MAb 5E12-B7 or saline. Data are the means of 8 female WT mice in each group. 2-way ANOVA for statistics. * p<0.05.

FIG. 17B is a graphical representation showing weights of white adipose tissue (WAT) from 4 depots expressed as percent of body weight of mice treated for eight weeks with MAb 5E12-B7 or saline. Data are the means of 8 female WT mice in each group. 2-way ANOVA for statistics. * p<0.05.

FIG. 18A is a graphical representation showing whole body fat mass of mice treated with MAb 5E12-B7 or saline obtained using dual-energy x-ray absorptiometry (DXA) scan conducted at an age of 9 (start of AB treatment), 14 (5 weeks of AB treatment) and 17 (8 weeks of AB treatment) weeks. Data are the means of 8 female WT mice in each group. T-test for statistics. *, p<0.05.

FIG. 18B is a graphical representation showing whole body fat mass expressed as a percent of body weight of mice treated with MAb 5E12-B7 or saline obtained using dual-energy x-ray absorptiometry (DXA) scan conducted at an age of 9 (start of AB treatment), 14 (5 weeks of AB treatment) and 17 (8 weeks of AB treatment) weeks. Data are the means of 8 female WT mice in each group. T-test for statistics. *, p<0.05.

FIG. 19A is a graphical representation showing sequences of heavy chain variable regions of antibodies 5E12 and humanized forms thereof (as indicated). Boxed regions contain CDRs (as indicated) as defined by the Kabat numbering system and the enhanced Chothia numbering system. CDRs defined by the Kabat numbering system are shown in bold. CDRs defined by the enhanced Chothia numbering system are underlined.

FIG. 19B is a graphical representation showing sequences of heavy chain variable regions of humanized forms of 5E12 (as indicated). Boxed regions contain CDRs (as indicated) as defined by the Kabat numbering system and the enhanced Chothia numbering system. CDRs defined by the Kabat numbering system are shown in bold. CDRs defined by the enhanced Chothia numbering system are underlined.

FIG. 19C is a graphical representation showing sequences of light chain variable regions of 5E12 and a humanized form thereof (as indicated). Boxed regions contain CDRs (as indicated) as defined by the Kabat numbering system and the enhanced Chothia numbering system. CDRs defined by the Kabat numbering system are shown in bold. CDRs defined by the enhanced Chothia numbering system are underlined.

FIG. 19D is a graphical representation showing sequences of heavy chain variable regions of human anti-NPY and PYY antibodies (as indicated). Boxed regions contain CDRs (as indicated) as defined by the Kabat numbering system and the enhanced Chothia numbering system. CDRs defined by the Kabat numbering system are shown in bold. CDRs defined by the enhanced Chothia numbering system are underlined.

FIG. 19E is a graphical representation showing sequences of light chain variable regions of human anti-NPY and PYY antibodies (as indicated). Boxed regions contain CDRs (as indicated) as defined by the Kabat numbering system and the enhanced Chothia numbering system. CDRs defined by the Kabat numbering system are shown in bold. CDRs defined by the enhanced Chothia numbering system are underlined.

FIG. 20 is a graphical representation showing percentage survival of mice administered breast cancer tumor cells and treated with either isotype control antibody or MAb 5E12-B7.

### DETAILED DESCRIPTION OF THE PREFERRED EMBODIMENT

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Each example of the present disclosure described herein is to be applied mutatis mutandis to each and every other example unless specifically stated otherwise.

Those skilled in the art will appreciate that the disclosure herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the disclosure, as described herein.

The present disclosure is performed without undue experimentation using, unless otherwise indicated, conventional techniques of molecular biology, microbiology, virology, recombinant DNA technology, peptide synthesis in solution, solid phase peptide synthesis, and immunology. Such procedures are described, for example, in Sambrook, Fritsch & Maniatis, Molecular Cloning: A Laboratory Manual, Cold Spring Harbor Laboratories, New York, Second Edition (1989), whole of Vols I, II, and III; Benny K. C. Lo, Antibody Engineering: Methods and Protocols, (2004) Humana Press, Vol. 248; DNA Cloning: A Practical Approach, Vols. I and II (D. N. Glover, ed., 1985), IRL Press, Oxford, whole of text; Oligonucleotide Synthesis: A Practical Approach (M. J. Gait, ed, 1984) IRL Press, Oxford, whole of text, and particularly the papers therein by Gait, pp1-22; Atkinson et al, pp35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984); Methods In Enzymology (S. Colowick and N. Kaplan, eds., Academic Press, Inc.), whole of series; J. F. Ramalho Ortigao, “The Chemistry of Peptide Synthesis” In: Knowledge database of Access to Virtual Laboratory website (Interactiva, Germany); Sakakibara, D., Teichman, J., Lien, E. Land Fenichel, R. L. (1976). Biochem. Biophys. Res. Commun. 73 336-342; Merrifield, R. B. (1963). J. Am. Chem. Soc. 85, 2149-2154; Barany, G. and Merrifield, R. B. (1979) in The Peptides (Gross, E. and Meienhofer, J. eds.), vol. 2, pp. 1-284, Academic Press, New York. 12. Wunsch, E., ed. (1974) Synthese von Peptiden in Houben-Weyls Metoden der Organischen Chemie (Miller, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474; Handbook of Experimental Immunology, Vols. I-IV (D. M. Weir and C. C. Blackwell, eds., 1986, Blackwell Scientific Publications); and Animal Cell Culture: Practical Approach, Third Edition (John R. W. Masters, ed., 2000), ISBN 0199637970, whole of text.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Key to Sequence Listing

SEQ ID NO: 1—amino acid sequence of human NPY
SEQ ID NO: 2—amino acid sequence of mouse NPY
SEQ ID NO: 3—amino acid sequence of human PYY
SEQ ID NO: 4—amino acid sequence of mouse PYY
SEQ ID NO: 5—amino acid sequence of human PP
SEQ ID NO: 6—amino acid sequence of mouse PP
SEQ ID NO: 7—amino acid sequence of 1p1
SEQ ID NO: 8—amino acid sequence of 1p2
SEQ ID NO: 9—amino acid sequence of 1p3
SEQ ID NO: 10—amino acid sequence of 1p4
SEQ ID NO: 11—amino acid sequence of 1p5
SEQ ID NO: 12—amino acid sequence of 1p6
SEQ ID NO: 13—amino acid sequence of 1p7
SEQ ID NO: 14—amino acid sequence of 1p8
SEQ ID NO: 15—amino acid sequence of 1p9
SEQ ID NO: 16—amino acid sequence of 1p10
SEQ ID NO: 17—amino acid sequence of 1p11
SEQ ID NO: 18—amino acid sequence of 1p12
SEQ ID NO: 19—amino acid sequence of 1p13
SEQ ID NO: 20—amino acid sequence of 1p14
SEQ ID NO: 21—amino acid sequence of 1p15
SEQ ID NO: 22—amino acid sequence of 1p16
SEQ ID NO: 23—amino acid sequence of 1p17
SEQ ID NO: 24—amino acid sequence of 1p18
SEQ ID NO: 25—amino acid sequence of 1p19
SEQ ID NO: 26—amino acid sequence of 1p20
SEQ ID NO: 27—amino acid sequence of 1p21
SEQ ID NO: 28—amino acid sequence of 1p22
SEQ ID NO: 29—amino acid sequence of 1p23
SEQ ID NO: 30—amino acid sequence of 1p24
SEQ ID NO: 31—amino acid sequence of 1p25
SEQ ID NO: 32—amino acid sequence of 1p26
SEQ ID NO: 33—amino acid sequence of 1p27
SEQ ID NO: 34—amino acid sequence of 1p28
SEQ ID NO: 35—amino acid sequence of 1p29
SEQ ID NO: 36—amino acid sequence of 1p30
SEQ ID NO: 37—amino acid sequence of 1p31
SEQ ID NO: 38—amino acid sequence of 1p32
SEQ ID NO: 39—amino acid sequence of 2p1
SEQ ID NO: 40—amino acid sequence of 2p2
SEQ ID NO: 41—amino acid sequence of 2p3
SEQ ID NO: 42—amino acid sequence of 2p4
SEQ ID NO: 43—amino acid sequence of 2p5
SEQ ID NO: 44—amino acid sequence of 2p6
SEQ ID NO: 45—amino acid sequence of 2p7
SEQ ID NO: 46—amino acid sequence of 2p8
SEQ ID NO: 47—amino acid sequence of 2p9
SEQ ID NO: 48—amino acid sequence of 2p10
SEQ ID NO: 49—amino acid sequence of 2p11
SEQ ID NO: 50—amino acid sequence of 2p12
SEQ ID NO: 51—amino acid sequence of 2p13
SEQ ID NO: 52—amino acid sequence of 2p14
SEQ ID NO: 53—amino acid sequence of 2p15
SEQ ID NO: 54—amino acid sequence of 2p16
SEQ ID NO: 55—amino acid sequence of 2p17
SEQ ID NO: 56—amino acid sequence of 2p18
SEQ ID NO: 57—amino acid sequence of 2p19
SEQ ID NO: 58—amino acid sequence of 2p20
SEQ ID NO: 59—amino acid sequence of 3p1
SEQ ID NO: 60—amino acid sequence of 3p2
SEQ ID NO: 61—amino acid sequence of 3p3
SEQ ID NO: 62—amino acid sequence of 3p4
SEQ ID NO: 63—amino acid sequence of 3p5
SEQ ID NO: 64—amino acid sequence of 3p6
SEQ ID NO: 65—amino acid sequence of 3p7
SEQ ID NO: 66—amino acid sequence of 3p8
SEQ ID NO: 67—amino acid sequence of 3p9
SEQ ID NO: 68—amino acid sequence of 3p10
SEQ ID NO: 69—amino acid sequence of 3p11
SEQ ID NO: 70—amino acid sequence of 3p12
SEQ ID NO: 71—amino acid sequence of 3p13
SEQ ID NO: 72—amino acid sequence of 3p14
SEQ ID NO: 73—amino acid sequence of 3p15
SEQ ID NO: 74—amino acid sequence of 3p16
SEQ ID NO: 75—amino acid sequence of 3p17
SEQ ID NO: 76—amino acid sequence of 3p18
SEQ ID NO: 77—amino acid sequence of 3p19
SEQ ID NO: 78—amino acid sequence of immunizing peptide
SEQ ID NO: 79—redundant amino acid sequence of epitope.
SEQ ID NO: 80—synthetic peptide 1
SEQ ID NO: 81—synthetic peptide 2
SEQ ID NO: 82—synthetic peptide 3
SEQ ID NO: 83—synthetic peptide 4
SEQ ID NO: 84—synthetic peptide 5
SEQ ID NO: 85—amino acid sequence of variable heavy chain 1
SEQ ID NO: 86—amino acid sequence of variable heavy chain 2
SEQ ID NO: 87—amino acid sequence of variable light chain 1
SEQ ID NO: 88—nucleotide sequence of variable heavy chain 1
SEQ ID NO: 89—nucleotide sequence of variable heavy chain 2
SEQ ID NO: 90—nucleotide sequence of variable light chain 1
SEQ ID NO: 91—amino acid sequence of V_{H} chain of humanized 5E12 IGHV1 69
SEQ ID NO: 92—amino acid sequence of V_{H} chain of humanized 5E12 IGHV1 46
SEQ ID NO: 93—amino acid sequence of V_{H} chain of mouse 5E12 PSE2
SEQ ID NO: 94—amino acid sequence of consensus sequence of V_{H} chain of humanized and mouse 5E12 antibodies
SEQ ID NO: 95—amino acid sequence of consensus sequence of V_{H} chain of humanized 5E12 antibodies
SEQ ID NO: 96—amino acid sequence of V_{L} chain of humanized 5E12 DPK9
SEQ ID NO: 97—amino acid sequence of V_{L} chain of mouse 5E12 PSE2
SEQ ID NO: 98—amino acid sequence of consensus sequence of V_{L} chain of humanized and mouse 5E12 antibodies
SEQ ID NO: 99—amino acid sequence of V_{H} chain of scFv-3
SEQ ID NO: 100—amino acid sequence of V_{H} chain of scFv-7
SEQ ID NO: 101—amino acid sequence of V_{H} chain of scFv-6
SEQ ID NO: 102—amino acid sequence of consensus sequence of V_{H} chain of human antibodies scFv-3, scFv-6 and scFv-7
SEQ ID NO: 103—amino acid sequence of V_{L} chain of scFv-3
SEQ ID NO: 104—amino acid sequence of V_{L} chain of scFv-7
SEQ ID NO: 105—amino acid sequence of V_{L} chain of scFv-6
SEQ ID NO: 106—amino acid sequence of consensus sequence of V_{L} chain of human antibodies scFv-3, scFv-6 and scFv-7
SEQ ID NO: 107—amino acid sequence of biotin-NPY₂₀₋₃₆ peptide
SEQ ID NO: 108—amino acid sequence of biotin-NPY peptide
SEQ ID NO: 109—amino acid sequence of biotin-hPYY peptide
SEQ ID NO: 110—amino acid sequence of biotin-hPP peptide
SEQ ID NO: 111—nucleotide sequence encoding scFv-3 antibody
SEQ ID NO: 112—amino acid sequence of scFv-3 antibody
SEQ ID NO: 113—nucleotide sequence encoding scFv-6 antibody
SEQ ID NO: 114—amino acid sequence of scFv-6 antibody
SEQ ID NO: 115—nucleotide sequence encoding scFv-7 antibody
SEQ ID NO: 116—amino acid sequence of scFv-7 antibody
SEQ ID NO: 117—nucleotide sequence encoding h5E12 IGHV1-46/DPK9 scFv
SEQ ID NO: 118—amino acid sequence of h5E12 IGHV1-46/DPK9 scFv
SEQ ID NO: 119—nucleotide sequence encoding h5E12 IGHV1-69/DPK9 scFv
SEQ ID NO: 120—amino acid sequence of h5E12 IGHV1-69/DPK9 scFv

In the foregoing sequences, SEQ ID NOs: 1-56, 58-82 and 107-110 are amidated peptides, i.e., comprise a C-terminal amine group.

### Selected Definitions

For the purposes of nomenclature and not limitation the amino acid sequence of human NPY is set forth in SEQ ID NO: 1 and the amino acid sequence of human NPY is set forth in SEQ ID NO: 2. Additional sequences of human NPY are set out in Genbank Gene Accession No. 4852 and additional sequences of mouse NPY are set out in Genbank Gene Accession No. 109648. In one example, the amino acid sequence of human NPY comprises a sequence set forth in SEQ ID NO: 1.

For the purposes of nomenclature and not limitation the amino acid sequence of human PYY is set forth in SEQ ID NO: 3 and the amino acid sequence of human PYY is set forth in SEQ ID NO: 4. Additional sequences of human PYY are set out in Genbank Gene Accession No. 5697 and additional sequences of mouse PYY are set out in Genbank Gene Accession No. 217212. In one example, the amino acid sequence of human PYY comprises a sequence set forth in SEQ ID NO: 3.

For the purposes of nomenclature and not limitation the amino acid sequence of human PP is set forth in SEQ ID NO: 5 and the amino acid sequence of human PP is set forth in SEQ ID NO: 6. Additional sequences of human PP are set out in Genbank Gene Accession No. 167780 and additional sequences of mouse PP are set out in Genbank Gene Accession No. 19064. In one example, the amino acid sequence of human PP comprises a sequence set forth in SEQ ID NO: 5.

The term “isolated protein” or “isolated polypeptide” is intended to mean a protein or polypeptide that by virtue of its origin or source of derivation is not associated with naturally-associated components that accompany it in its native state; is substantially free of other proteins from the same source. A protein may be rendered substantially free of naturally associated components or substantially purified by isolation, using protein purification techniques known in the art. By “substantially purified” is meant the protein is substantially free of contaminating agents, e.g., at least about 70% or 75% or 80% or 85% or 90% or 95% or 96% or 97% or 98% or 99% free of contaminating agents.

The term “recombinant” shall be understood to mean the product of artificial genetic recombination. Accordingly, in the context of a recombinant protein comprising an antibody antigen binding domain, this term does not encompass an antibody naturally-occurring within a subject's body that is the product of natural recombination that occurs during B cell maturation. However, if such an antibody is isolated, it is to be considered an isolated protein comprising an antibody antigen binding domain. Similarly, if nucleic acid encoding the protein is isolated and expressed using recombinant means, the resulting protein is a recombinant protein comprising an antibody antigen binding domain. A recombinant protein also encompasses a protein expressed by artificial recombinant means when it is within a cell, tissue or subject, e.g., in which it is expressed.

As used herein, the term “binds” in reference to the interaction of a NPY and PYY-binding protein with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabeled “A”), in a reaction containing labeled “A” and the antibody, will reduce the amount of labeled “A” bound to the antibody.

As used herein, the term “specifically binds” or “binds specifically” shall be taken to mean that a protein reacts or associates more frequently, more rapidly, with greater duration and/or with greater affinity with a recited protein or proteins (e.g., NPY and PYY) than it does with another protein (e.g., with PP). For example, a protein that specifically binds to PYY and NPY binds with greater affinity, avidity, more readily, and/or with greater duration than it binds PP. In this regard, the degree of greater affinity, avidity, readiness, and/or with duration will depend on the application of the protein. For example, for detection/diagnostic/prognostic purposes the degree of specificity should be sufficiently high to permit quantification (where required). For therapeutic/prophylactic applications, the degree of specificity should be sufficient to provide a therapeutic/prophylactic effect without serious adverse effects resulting from cross-reactivity of the protein and/or without sufficient cross-reactivity to significantly reduce PP biological activity in a subject. In one example, a protein that specifically binds to NPY and PYY does not significantly or detectably bind to PP. It is also to be understood by reading this definition that “specific binding” does not necessarily require exclusive binding, this is encompassed by the term “selective binding”. Generally, but not necessarily, reference to binding means specific binding. In one example, “specific binding” of a NPY and PYY-binding protein of the disclosure to an antigen, means that the protein binds to the antigen with a K_{D} of 1×10⁻⁷M or less, such as 5×10⁻⁸M or less, for example 2×10⁻⁸M or less, such as, 1.5×10⁻⁸M or less or 1×10⁻⁸ nM or less.

As used herein, the term “does not significantly bind” shall be understood to mean that the level of binding to an antigen or epitope of a protein of the present disclosure is not statistically significantly higher than background, e.g., the level of binding signal detected in the absence of the protein and/or in the presence of a negative control protein (e.g., an isotype control antibody) and/or the level of binding detected in the presence of a negative control antigen or epitope. In one example, the level of binding is assessed by ELISA, e.g., indirect ELISA.

As used herein, the term “does not detectably bind” shall be understood to mean that a protein, e.g., an antibody, binds to an antigen or epitope at a level of 30% or 25% or 20% or 15% or 10% or less than the signal obtained with human or mouse NPY or PYY. Alternatively, or additionally, performing an ELISA with the NPY and PYY-binding protein “does not detectably bind” to an antigen or epitope and detecting binding by detecting signal intensity at OD 450 nm results in a signal of 0.5 or less. The level of binding can be detected using ELISA, e.g., indirect ELISA or Biacore analysis in which the protein is immobilized and contacted with an antigen or epitope.

The term “binds to an epitope” means that an antibody binds to amino acids within the sequence of the recited epitope. This term does not mean that the antibody binds to each and every amino acid recited, only that one or more of the recited amino acids are necessary for antibody binding.

As used herein, the term “epitope” (syn. “antigenic determinant”) shall be understood to mean a region of NPY and PYY to which a protein comprising an antibody variable region binds. This term is not necessarily limited to the specific residues or structure to which the protein makes contact. For example, this term includes the region spanning amino acids contacted by the protein and/or at least 5-10 or 2-5 or 1-3 amino acids outside of this region. In some examples, the epitope is a linear series amino acids. However, the epitope is not restricted to only amino acid side-chains. For example, a protein described herein binds to a peptide comprising an amidated terminus but does not significantly bind to the same sequence of amino acids with a carboxy terminus. Sequences contained within exemplary epitopes in NPY/PYY are described herein.

As used herein, use of the term “C-terminus” in the context of an epitope or peptide or antigen will be understood to mean that the recited sequence is positioned such that the last amino acid in the recited sequence is the carboxy terminal amino acid in the epitope or peptide or antigen.

As used herein the term “amidated” or “X-amide” (wherein “X” is an amino acid) shall be understood to mean carboxy group of an amino acid or on the C-terminus of a peptide is replaced with an amide group. For example, the amino acid or peptide is α-amidated.

The term “NPY and PYY-binding protein” shall be taken to include a single polypeptide chain (i.e., a series of contiguous amino acids linked by peptide bonds), or a series of polypeptide chains covalently or non-covalently linked to one another (i.e., a polypeptide complex) capable of binding to NPY and PYY in the manner described and/or claimed herein. For example, the series of polypeptide chains can be covalently linked using a suitable chemical or a disulphide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

For the purposes for the present disclosure, the term “antibody” includes a protein capable of specifically binding to one or a few closely related antigens (e.g., NPY and PYY) by virtue of an antigen binding domain contained within a Fv. This term includes four chain antibodies (e.g., two light chains and two heavy chains), recombinant or modified antibodies (e.g., chimeric antibodies, humanized antibodies, human antibodies, CDR-grafted antibodies, primatized antibodies, de-immunized antibodies, synhumanized antibodies, half antibodies, and bispecific antibodies). An antibody generally comprises constant domains, which can be arranged into a constant region or constant fragment or fragment crystallizable (Fc). Exemplary forms of antibodies comprise a four-chain structure as their basic unit. Full-length antibodies comprise two heavy chains (˜50-70 kDa) covalently linked and two light chains (˜23 kDa each). A light chain generally comprises a variable region (if present) and a constant domain and in mammals is either a κ light chain or a λ light chain. A heavy chain generally comprises a variable region and one or two constant domain(s) linked by a hinge region to additional constant domain(s). Heavy chains of mammals are of one of the following types α, δ, □, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (V_{H} or V_{L} wherein each are ˜110 amino acids in length) and one or more constant domains at the C-terminus. The constant domain of the light chain (C_{L} which is ˜110 amino acids in length) is aligned with and disulfide bonded to the first constant domain of the heavy chain (C_{H}1 which is 330-440 amino acids in length). The light chain variable region is aligned with the variable region of the heavy chain. The antibody heavy chain can comprise 2 or more additional C_{H} domains (such as, C_{H}2, C_{H}3 and the like) and can comprise a hinge region between the C_{H}1 and C_{H}2 constant domains. Antibodies can be of any type (e.g., IgG, IgE, IgM, IgD, IgA, and IgY), class (e.g., IgG₁, IgG₂, IgG₃, IgG₄, IgA₁ and IgA₂) or subclass. In one example, the antibody is a murine (mouse or rat) antibody or a primate (such as, human) antibody. In one example, the antibody is humanized, synhumanized, chimeric, CDR-grafted or deimmunized.

As used herein, “variable region” refers to the portions of the light and/or heavy chains of an antibody as defined herein that is(are) capable of specifically binding to an antigen and, includes amino acid sequences of complementarity determining regions (CDRs); i.e., CDR1, CDR2, and CDR3, and framework regions (FRs). For example, the variable region comprises three or four FRs (e.g., FR1, FR2, FR3 and optionally FR4) together with three CDRs. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region domain (V_{H} or V_{L}) typically has three CDR regions identified as CDR1, CDR2 and CDR3. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”. In another example, the amino acid positions assigned to CDRs and FRs are defined according to the Enhanced Chothia Numbering Scheme (http://www.bioinfo.org.uk/mdex.html). According to the numbering system of Kabat, V_{H} FRs and CDRs are positioned as follows: residues 1-30 (FR1), 31-35 (CDR1), 36-49 (FR2), 50-65 (CDR2), 66-94 (FR3), 95-102 (CDR3) and 103-113 (FR4). According to the numbering system of Kabat, V_{L} FRs and CDRs are positioned as follows: residues 1-23 (FR1), 24-34 (CDR1), 35-49 (FR2), 50-56 (CDR2), 57-88 (FR3), 89-97 (CDR3) and 98-107 (FR4). The present disclosure is not limited to FRs and CDRs as defined by the Kabat numbering system, but includes all numbering systems, including the canonical numbering system or of Chothia and Lesk J. Mol Biol. 196:901-917, 1987; Chothia et al. Nature 342, 877-883, 1989; and/or Al-Lazikani et al., J Mol Biol 273, 927-948, 1997; the numbering system of Honnegher and Plükthun J. Mol. Biol., 309: 657-670, 2001; or the IMGT system discussed in Giudicelli et al., Nucleic Acids Res., 25: 206-211 1997. In one example, the CDRs are defined according to the Kabat numbering system. Optionally, heavy chain CDR2 according to the Kabat numbering system does not comprise the five C-terminal amino acids listed herein or any one or more of those amino acids are substituted with another naturally-occurring amino acid. In an additional, or alternative, option, light chain CDR1 does not comprise the four N-terminal amino acids listed herein or any one or more of those amino acids are substituted with another naturally-occurring amino acid. In this regard, Padlan et al., FASEB J., 9: 133-139, 1995 established that the five C-terminal amino acids of heavy chain CDR2 and/or the four N-terminal amino acids of light chain CDR1 are not generally involved in antigen binding.

“Framework regions” (FRs) are those variable region residues other than the CDR residues.

As used herein, the term “Fv” shall be taken to mean any protein, whether comprised of multiple polypeptides or a single polypeptide, in which a V_{L} and a V_{H} associate and form a complex capable of specifically binding to an antigen. The V_{H} and the V_{L} which form the antigen binding domain can be in a single polypeptide chain or in different polypeptide chains. Furthermore, an Fv of the disclosure (as well as any protein of the disclosure) may have multiple antigen binding domains which may or may not bind the same antigen. This term shall be understood to encompass fragments directly derived from an antibody as well as proteins corresponding to such a fragment produced using recombinant means. In some examples, the V_{H} is not linked to a heavy chain constant domain (C_{H}) 1 and/or the V_{L} is not linked to a light chain constant domain (C_{L}). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., C_{H}2 or C_{H}3 domain, e.g., a minibody. A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a V_{H} and a single constant domain. Two Fab′ fragments are obtained per antibody treated in this manner. A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab₂” fragment is a recombinant fragment comprising two Fab fragments linked using, for example a leucine zipper or a C_{H}3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

The term “competitively inhibits” shall be understood to mean that a NPY and PYY-binding protein of the disclosure reduces or prevents binding of a recited antibody to NPY or PYY. This may be due to the protein (or variable region or Fv thereof) and antibody binding to the same or an overlapping epitope. It will be apparent from the foregoing that the protein need not completely inhibit binding of the antibody, rather it need only reduce binding by a statistically significant amount, for example, by at least about 10% or 20% or 30% or 40% or 50% or 60% or 70% or 80% or 90% or 95%. Methods for determining competitive inhibition of binding are known in the art and/or described herein. For example, the antibody is exposed to NPY or PYY either in the presence or absence of the protein. If less antibody binds in the presence of the protein than in the absence of the protein, the protein is considered to competitively inhibit binding of the antibody. In one example, the competitive inhibition of binding is caused by the epitope bound by the protein on NPY or PYY overlapping with the antigen binding domain of the antibody.

“Overlapping” in the context of two epitopes shall be taken to mean that two epitopes share a sufficient number of amino acid residues to permit a protein (or antigen binding domain thereof) that binds to one epitope to competitively inhibit the binding of a protein (or antigen binding domain) that binds to the other epitope. For example, the “overlapping” epitopes share at least 1 or 2 or 3 or 4 or 5 or 6 or 7 or 8 or 9 or 20 amino acids.

As used herein, the term “neutralize” shall be taken to mean that a NPY and PYY-binding protein is capable of reducing or preventing NPY and/or PYY-mediated activity in a cell. Methods for determining neutralization are known in the art and/or described herein.

As used herein, the term “condition” refers to a disruption of or interference with normal function, and is not to be limited to any specific condition, and will include diseases or disorders.

As used herein, a “NPY and/or PYY-associated condition” refers to any condition that is caused by or associated with NPY and/or PYY. The skilled artisan will be readily able to determine such conditions based on the disclosure herein. In this regard, in some examples the condition is anorexia, unintended weight loss or a wasting condition or cancer.

As used herein, the term “wasting disorder” refers to a disorder which involves, results at least in part from, or includes loss of weight, muscle atrophy, fatigue, weakness in someone who is not actively trying to lose weight. Wasting disorders are commonly characterized by inadvertent and/or uncontrolled (in the absence of medical intervention) loss of muscle and/or fat. The term encompasses cachexia or other forms of wasting, e.g., denervation-induced wasting.

As used herein, the term “cachexia” will be understood to refer to metabolic condition associated with an underlying (or another) condition, wherein cachexia is characterized by loss of body weight and loss of muscle with or without loss of fat mass. Cachexia is generally associated with increased protein catabolism due to underlying disease(s). Contributory factors to the onset of cachexia are anorexia and metabolic alterations (e.g., increased inflammatory status, increased muscle proteolysis and impaired carbohydrate, protein and lipid metabolism). A prominent clinical feature of cachexia is weight loss in adults (optionally, corrected for fluid retention) or growth failure in children (excluding endocrine disorders). Anorexia, inflammation, insulin resistance and increased muscle protein breakdown are frequently associated with cachexia. Cachexia is distinct from starvation, primary depression, malabsorption and hyperthyroidism and is associated with increased morbidity. Cachexia can be associated with or result from (directly or indirectly) various underlying disorders including cancer, metabolic acidosis (from decreased protein synthesis and increased protein catabolism), certain infectious diseases (e.g. bacterial infections, including tuberculosis, AIDS), some autoimmune disorders, addiction to drugs such as amphetamines or cocaine, chronic alcoholism and/or cirrhosis of the liver, chronic inflammatory disorders, anorexia, neurological conditions and/or neurodegenerative disease. In one example, cachexia is cancer cachexia (cachexia associated with cancer). In other examples, muscle wasting and/or unintended body weight loss associated with neurological conditions, immobility or impaired mobility due to various diseases such as neurodegenerative disease, multiple sclerosis, spinal cord injury, are included in the term. Cachexia can be diagnosed based on one or more of the following:
Weight loss of at least 5% over a period of six months (in the absence of starvation);
A BMI<20 together with weight loss; or
Appendicular skeletal muscle index consistent with sarcopenia (males<7.26 kg/m²; females<5.45 kg/m²) together with weight loss.

As used herein, the term “pre-cachexia” will be understood to mean a condition associated with an underlying condition (e.g., chronic condition) and characterized by unintentional weight loss of less than about 5% of a subject's body weight; and a chronic or recurrent systemic inflammatory response.

As used herein, the term “unintended body weight loss” refers to a condition where the subject is incapable of maintaining a healthy body weight or loses a considerable amount of body weight, without actually attempting to reduce body weight. For example a body mass index of less than 18.5 (or any another BMI range defined by a medical specialist) is considered underweight.

As used herein, the terms “preventing”, “prevent” or “prevention” include administering a compound to thereby stop or hinder the development of at least one symptom of a condition. This term also encompasses treatment of a subject in remission to prevent or hinder relapse.

As used herein, the terms “treating”, “treat” or “treatment” include administering a compound to thereby reduce or eliminate at least one symptom of a specified condition.

As used herein, the term “subject” shall be taken to mean any animal, such as, a mammal. In one example, the mammal is a human or non-human primate. In one example, the mammal is a human.

Reference herein to a “sample” should be understood as a reference to any sample derived from a subject such as, but not limited to, a body fluid (e.g., blood or blood fraction such as serum or plasma, tears, urine, synovial fluid or cerebrospinal fluid), cellular material (e.g. tissue aspirate), tissue biopsy specimens or surgical specimens. In some examples, the “sample” is any one or more of serum, plasma, peripheral blood mononuclear cells (PBMC), or a buffy coat fraction.

The term “expression construct” is to be taken in its broadest context and includes a nucleic acid comprising one or more promoter sequences operably linked with one or more nucleic acids as described herein.

The term “expression vector” refers to a nucleic acid comprising an expression construct that is additionally capable of maintaining and or replicating nucleic acid in an expressible format. For example, an expression vector may comprise a plasmid, bacteriophage, phagemid, cosmid, virus sub-genomic or genomic fragment. Selection of appropriate vectors is within the knowledge of those having skill in the art.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter. A promoter can be operably linked to numerous nucleic acids, e.g., through an internal ribosome entry site.

By “structurally related to glutamine” is meant an amino acid that is considered similar to glutamine based on the BLOSUM62 matrix and/or PAM250 matix and includes glutamine.

### Proteins Comprising Antibody Variable Region(s)

### Antibodies

### Immunization-Based Methods

To generate antibodies, NPY or PYY or a peptide comprising a sequence conserved in NPY and PYY (e.g., a sequence set forth in SEQ ID NO: 78) or an epitope bearing fragment or portion thereof or a modified form thereof or nucleic acid encoding same, optionally formulated with any suitable or desired adjuvant and/or pharmaceutically acceptable carrier, is administered to a subject (for example, a non-human animal subject, such as, a mouse, a rat, a chicken etc.) in the form of an injectable composition. Exemplary non-human animals are mammals, such as murine animals (e.g., rats or mice). Injection may be intranasal, intramuscular, sub-cutaneous, intravenous, intradermal, intraperitoneal, or by other known route. Optionally, the NPY/PYY/peptide or epitope bearing fragment or portion thereof or a nucleic acid encoding same is administered numerous times. Means for preparing and characterizing antibodies are known in the art (See, e.g., Antibodies: A Laboratory Manual, Cold Spring Harbor Laboratory, 1988).

The production of polyclonal antibodies may be monitored by sampling blood of the immunized animal at various points following immunization. A second, booster injection, may be given, if required to achieve a desired antibody titer. The process of boosting and titering is repeated until a suitable titer is achieved. When a desired level of immunogenicity is obtained, the immunized animal is bled and the serum isolated and stored, and/or the animal is used to generate monoclonal antibodies (mAbs).

Monoclonal antibodies are exemplary antibodies contemplated by the present disclosure. Generally, production of monoclonal antibodies involves, immunizing a subject (e.g., a rodent, e.g., mouse or rat) with a peptide or a nucleic acid encoding same under conditions sufficient to stimulate antibody producing cells. In some examples, a mouse genetically-engineered to express human immunoglobulin proteins and not express murine immunoglobulin proteins, is immunized to produce an antibody (e.g., as described in PCT/US2007/008231 and/or Lonberg et al., Nature 368 (1994): 856-859). Following immunization, antibody producing somatic cells (e.g., B lymphocytes) are fused with immortal cells, e.g., immortal myeloma cells. Various methods for producing such fused cells (hybridomas) are known in the art and described, for example, in Kohler and Milstein, Nature 256, 495-497, 1975. The hybridoma cells can then be cultured under conditions sufficient for antibody production.

The present disclosure contemplates other methods for producing antibodies, e.g., ABL-MYC technology (as described, for example in Largaespada et al, Curr. Top. Microbiol. Immunol, 166, 91-96. 1990).

Suitable antibodies are then selected based on methods described herein.

### Library-Based Methods

The present disclosure also encompasses screening of libraries of antibodies or proteins comprising antigen binding domains thereof (e.g., comprising variable regions thereof) to identify NPY and PYY-binding protein of the disclosure.

Examples of libraries contemplated by this disclosure include naïve libraries (from unchallenged subjects), immunized libraries (from subjects immunized with an antigen) or synthetic libraries. Nucleic acid encoding antibodies or regions thereof (e.g., variable regions) are cloned by conventional techniques (e.g., as disclosed in Sambrook and Russell, eds, Molecular Cloning: A Laboratory Manual, 3rd Ed, vols. 1-3, Cold Spring Harbor Laboratory Press, 2001) and used to encode and display proteins using a method known in the art. Other techniques for producing libraries of proteins are described in, for example in U.S. Pat. No. 6,300,064 (e.g., a HuCAL library of Morphosys AG); U.S. Pat. No. 5,885,793; U.S. Pat. No. 6,204,023; U.S. Pat. No. 6,291,158; or U.S. Pat. No. 6,248,516. Another exemplary library is described in WO2011/047442.

The NPY and PYY-binding proteins according to the disclosure may be soluble secreted proteins or may be presented as a fusion protein on the surface of a cell, or particle (e.g., a phage or other virus, a ribosome or a spore). Various display library formats are known in the art. For example, the library is an in vitro display library (e.g., a ribosome display library, a covalent display library or a mRNA display library, e.g., as described in U.S. Pat. No. 7,270,969). In yet another example, the display library is a phage display library wherein proteins comprising antigen binding domains of antibodies are expressed on phage, e.g., as described in U.S. Pat. No. 6,300,064; U.S. Pat. No. 5,885,793; U.S. Pat. No. 6,204,023; U.S. Pat. No. 6,291,158; or U.S. Pat. No. 6,248,516. Other phage display methods are known in the art and are contemplated by the present disclosure. Similarly, methods of cell display are contemplated by the disclosure, e.g., bacterial display libraries, e.g., as described in U.S. Pat. No. 5,516,637; yeast display libraries, e.g., as described in U.S. Pat. No. 6,423,538 or a mammalian display library.

Methods for screening display libraries are known in the art. In one example, a display library of the present disclosure is screened using affinity purification, e.g., as described in Scopes (In: Protein purification: principles and practice, Third Edition, Springer Verlag, 1994). Methods of affinity purification typically involve contacting proteins comprising antigen binding domains displayed by the library with a target antigen (e.g., NPY and PYY) and, following washing, eluting those domains that remain bound to the antigen.

Any variable regions or scFvs identified by screening are readily modified into a complete antibody, if desired. Exemplary methods for modifying or reformatting variable regions or scFvs into a complete antibody are described, for example, in Jones et al., J Immunol Methods. 354:85-90, 2010; or Jostock et al., J Immunol Methods, 289: 65-80, 2004. Alternatively, or additionally, standard cloning methods are used, e.g., as described in Ausubel et at (In: Current Protocols in Molecular Biology. Wiley Interscience, ISBN 047 150338, 1987), and/or (Sambrook et at (In: Molecular Cloning: Molecular Cloning: A Laboratory Manual, Cold Spring Harbor Laboratories, New York, Third Edition 2001).

### Deimmunized, Chimeric, Humanized, Synhumanized, Primatized and Human NPY and PYY-Binding Proteins

The NPY and PYY-binding proteins of the present disclosure may be may be humanized proteins.

The term “humanized protein” shall be understood to refer to a protein comprising a human-like variable region, which includes CDRs from an antibody from a non-human species (e.g., mouse or rat or non-human primate) grafted onto or inserted into FRs from a human antibody (this type of antibody is also referred to a “CDR-grafted antibody”). Humanized proteins also include proteins in which one or more residues of the human protein are modified by one or more amino acid substitutions and/or one or more FR residues of the human protein are replaced by corresponding non-human residues. Humanized proteins may also comprise residues which are found in neither the human antibody or in the non-human antibody. Any additional regions of the protein (e.g., Fc region) are generally human. Humanization can be performed using a method known in the art, e.g., U.S. Pat. No. 5,225,539, U.S. Pat. No. 6,054,297, U.S. Pat. No. 7,566,771 or U.S. Pat. No. 5,585,089. The term “humanized protein” also encompasses a super-humanized protein, e.g., as described in U.S. Pat. No. 7,732,578.

In one example, a humanized NPY and PYY-binding protein comprises the regions between 27d and 34, 50 and 55, and 89 and 96 in a light chain sequence disclosed herein; and 31 and 35b, 50 and 58, and 95 and 101 in a heavy chain sequence disclosed herein (numbering according to the Kabat numbering system). In this regard, Padlan et al., FASEB J, 9: 133-139, 1995 presents evidence that these regions are those most likely to bind or contact antigen.

The NPY and PYY-binding proteins of the present disclosure may be human proteins. The term “human protein” as used herein refers to proteins having variable and, optionally, constant antibody regions found in humans, e.g. in the human germline or somatic cells or from libraries produced using such regions. The “human” proteins can include amino acid residues not encoded by human sequences, e.g. mutations introduced by random or site directed mutations in vitro (in particular mutations which involve conservative substitutions or mutations in a small number of residues of the protein, e.g. in 1, 2, 3, 4 or 5 of the residues of the protein). These “human proteins” do not necessarily need to be generated as a result of an immune response of a human, rather, they can be generated using recombinant means (e.g., screening a phage display library) and/or by a transgenic animal (e.g., a mouse) comprising nucleic acid encoding human antibody constant and/or variable regions and/or using guided selection (e.g., as described in or U.S. Pat. No. 5,565,332). This term also encompasses affinity matured forms of such antibodies. For the purposes of the present disclosure, a human protein will also be considered to include a protein comprising FRs from a human antibody or FRs comprising sequences from a consensus sequence of human FRs and in which one or more of the CDRs are random or semi-random, e.g., as described in U.S. Pat. No. 6,300,064 and/or U.S. Pat. No. 6,248,516.

The NPY and PYY-binding proteins of the present disclosure may be synhumanized proteins. The term “synhumanized protein” refers to a protein prepared by a method described in WO2007/019620. A synhumanized NPY and PYY-binding protein includes a variable region of an antibody, wherein the variable region comprises FRs from a New World primate antibody variable region and CDRs from a non-New World primate antibody variable region. For example, a synhumanized NPY and PYY-binding protein includes a variable region of an antibody, wherein the variable region comprises FRs from a New World primate antibody variable region and CDRs from a mouse or rat antibody. In one example, the synhumanized NPY and PYY-binding protein is a NPY and PYY-binding antibody in which one or both of the variable regions are synhumanized.

The NPY and PYY-binding proteins of the present disclosure may be primatized proteins. A “primatized protein” comprises variable region(s) from an antibody generated following immunization of a non-human primate (e.g., a cynomolgus macaque). Optionally, the variable regions of the non-human primate antibody are linked to human constant regions to produce a primatized antibody. Exemplary methods for producing primatized antibodies are described in U.S. Pat. No. 6,113,898.

In one example a NPY and PYY-binding protein of the disclosure is a chimeric protein. The term “chimeric proteins” refers to proteins in which an antigen binding domain is from a particular species (e.g., murine, such as mouse or rat) or belonging to a particular antibody class or subclass, while the remainder of the protein is from a protein derived from another species (such as, for example, human or non-human primate) or belonging to another antibody class or subclass. In one example, a chimeric protein is a chimeric antibody comprising a V_{H} and/or a V_{L} from a non-human antibody (e.g., a murine antibody) and the remaining regions of the antibody are from a human antibody. The production of such chimeric proteins is known in the art, and may be achieved by standard means (as described, e.g., in U.S. Pat. No. 6,331,415; U.S. Pat. No. 5,807,715; U.S. Pat. No. 4,816,567 and U.S. Pat. No. 4,816,397).

The present disclosure also contemplates a deimmunized NPY and PYY-binding protein, e.g., as described in WO02000/34317 and WO2004/108158. De-immunized antibodies and proteins have one or more epitopes, e.g., B cell epitopes or T cell epitopes removed (i.e., mutated) to thereby reduce the likelihood that a subject will raise an immune response against the antibody or protein. For example, a NPY and PYY-binding protein of the disclosure is analyzed to identify one or more B or T cell epitopes and one or more amino acid residues within the epitope is mutated to thereby reduce the immunogenicity of the protein.

### Other NPY and PYY-Binding Proteins Comprising an Antigen Binding Domain

The present disclosure also contemplates other NPY and PYY-binding proteins comprising a variable region or antigen binding domain of an antibody, such as:
(i) a single-domain antibody, which is a single polypeptide chain comprising all or a portion of the V_{H} or a V_{L} of an antibody (see, e.g., U.S. Pat. No. 6,248,516);
(ii) diabodies, triabodies and tetrabodies, e.g., as described in U.S. Pat. No. 5,844,094 and/or US2008152586;
(iii) scFvs, e.g., as described in U.S. Pat. No. 5,260,203;
(iv) minibodies, e.g., as described in U.S. Pat. No. 5,837,821;
(v) heteroconjugate proteins, e.g., as described in U.S. Pat. No. 4,676,980;
(vi) heteroconjugate proteins produced using a chemical cross-linker, e.g., as described in U.S. Pat. No. 4,676,980;
(vii) Fab′-SH fragments, e.g., as described in Shalaby et al, J. Exp. Med., 175: 217-225, 1992; or
(viii) Fab₃ (e.g., as described in EP19930302894).

### Exemplary NPY and PYY-Binding Proteins

Exemplary variable region containing NPY and PYY-binding proteins produced by the inventors are described in Table 1.

TABLE 1
Sequences Of Exemplary NPY And PYY-Binding Proteins

| | | V_{H} | |
|---|---|---|---|
| | | amino acid | V_{L} amino acid |
| | Antibody Name | SEQ ID NO | SEQ ID NO |
| 1 | 5E12 | 85 or 86 | 87 |
| 2 | 5E12 IGHV1 69 DPK9 | 91 | 96 |
| 3 | 5E12 IGHV1 46 DPK9 | 92 | 96 |
| 4 | scFv-3 | 99 | 103 |
| 5 | scFv-6 | 101 | 105 |
| 6 | scFv-7 | 100 | 104 |

### Multispecific Binding Proteins

Therapeutic/prophylactic methods described herein also make use of multispecific binding proteins. Exemplary multi-specific binding proteins comprise a plurality of variable regions each capable of binding to a different protein or different epitope within a protein. For example, the multi-specific binding protein comprises a variable domain that binds to NPY and a variable domain that binds to PYY. In another example, the multi-specific binding protein comprises a variable domain that binds to one or more forms of NPY, a variable domain that binds to one or more additional forms of NPY and a variable domain that binds to PYY. In another example, the multi-specific binding protein comprises a variable domain that binds to one or more forms of PYY, a variable domain that binds to one or more additional forms of PYY and a variable domain that binds to NPY.

Exemplary multi-specific binding proteins include:
dual variable domain immunoglobulins, such as those comprising at least two domain antibodies that each bind to different proteins, e.g., as described in WO2007/024715;
biologically active antibody dimers, e.g., as reported in U.S. Pat. No. 6,897,044;
a multivalent Fv antibody construct having at least four variable domains which are linked with each other via peptide linkers, e.g., as described in U.S. Pat. No. 7,129,330;
dimeric or multimeric antigen binding structures as described in US 20050079170;
tri- or tetra-valent multi-specific antigen-binding proteins comprising three or four Fab fragments bound to each other covalently by a connecting structure, which protein is not a natural immunoglobulin, e.g., as described in U.S. Pat. No. 6,511,663;
tetravalent bispecific antibodies as described in WO2006/020258;
bispecific tetravalent receptors are reported in U.S. Pat. No. 5,959,083;
engineered antibodies with three or more functional antigen binding sites as described in WO2001/077342.

An exemplary form of multispecific binding protein of the disclosure comprises a scFv that binds to NPY, a scFv that binds to PYY and an antibody Fc region or heavy chain constant region. The components can be arranged as scFv₁-Fc-scFv₂ or scFv₁-scFv₂-Fc or scFv₂-scFv₁-Fc (where Fc can be an Fc region of an antibody or an antibody constant region and scFv₁ bind to NPY and scFv₂ binds to PYY). One or both of the scFv can be replaced with domain antibodies. Additional scFv (or domain antibodies) can be added (e.g., fused to an existing scFv (or domain antibody) or to a constant region) to increase the number of proteins bound by the binding protein).

Another exemplary form of a multispecific binding protein of the disclosure comprises scFv that binds to NPY fused to a light chain constant region and a scFv that binds to PYY fused to a heavy chain constant region. One or both of the scFv can be replaced with domain antibodies. Additional scFv (or domain antibodies) can be added (e.g., fused to an existing scFv (or domain antibody) or to a constant region) to increase the number of proteins bound by the binding protein).

A further exemplary form of a multispecific binding protein of the disclosure is a multispecific diabody, triabody or tetrabody, (see, e.g., Mack et al., Proc. Natl. Acad. Sci., 92: 7021-7025, 1995).

Exemplary sources of variable regions that bind NPY for producing multi-specific binding proteins include, for example, an antibody described in Walter et al., Peptides, 15: 607-613, 1994 or as are commercially available, e.g., from Abcam, Thermo Scientific Pierce or Bachem. Exemplary sources of variable regions that bind PYY (or specific forms thereof) for producing multi-specific binding proteins include, for example, as described in WO/2006/108234 or as are commercially available, e.g., from Pierce, Abcam or Abnova.

### Constant Domain Fusions

The present disclosure encompasses a NPY and PYY-binding protein comprising an antigen binding domain of an antibody and a constant region or Fc or a domain thereof, e.g., C_{H}2 and/or C_{H}3 domain. Suitable constant regions and/or domains will be apparent to the skilled artisan and/or the sequences of such polypeptides are readily available from publicly available databases. Kabat et at also provide description of some suitable constant regions/domains.

Constant regions and/or domains thereof are useful for providing biological activities such as, dimerization, extended serum half life (e.g., by binding to FcRn), antigen dependent cell cytotoxicity (ADCC), complement dependent cytotoxicity (CDC, antigen dependent cell phagocytosis (ADCP).

The present disclosure also contemplates NPY and PYY-binding proteins comprising mutant constant regions or domains, e.g., as described in U.S. Pat. No. 7,217,797; U.S. Pat. No. 7,217,798; or US20090041770 (having increased half-life) or US2005037000 (increased ADCC).

### Stabilized NPY and PYY-Binding Proteins

Neutralizing NPY and PYY-binding proteins of the present disclosure can comprise an IgG4 constant region or a stabilized IgG4 constant region. The term “stabilized IgG4 constant region” will be understood to mean an IgG4 constant region that has been modified to reduce Fab arm exchange or the propensity to undergo Fab arm exchange or formation of a half-antibody or a propensity to form a half antibody. “Fab arm exchange” refers to a type of protein modification for human IgG4, in which an IgG4 heavy chain and attached light chain (half-molecule) is swapped for a heavy-light chain pair from another IgG4 molecule. Thus, IgG4 molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG4 antibody dissociates to form two molecules each containing a single heavy chain and a single light chain.

In one example, a stabilized IgG4 constant region comprises a proline at position 241 of the hinge region according to the system of Kabat (Kabat et al., Sequences of Proteins of Immunological Interest Washington D.C. United States Department of Health and Human Services, 1987 and/or 1991). This position corresponds to position 228 of the hinge region according to the EU numbering system (Kabat et al., Sequences of Proteins of Immunological Interest Washington D.C. United States Department of Health and Human Services, 2001 and Edelman et al., Proc. Natl. Acad. USA, 63, 78-85, 1969). In human IgG4, this residue is generally a serine. Following substitution of the serine for proline, the IgG4 hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG1 according to the numbering system of Kabat. Hinge regions of other IgG isotypes may be aligned with the IgG1 sequence by placing the first and last cysteine residues forming inter-heavy chain disulphide (S—S) bonds in the same positions (see for example WO2010/080538).

### Mutant NPY and PYY-Binding Proteins

The present disclosure also provides a NPY and PYY-binding protein or a nucleic acid encoding same having at least 80% identity to a sequence disclosed herein. In one example, a NPY and PYY-binding protein or nucleic acid of the disclosure comprises sequence at least about 85% or 90% or 95% or 97% or 98% or 99% identical to a sequence disclosed herein, wherein the protein specifically binds to NPY and PYY.

Alternatively, or additionally, the NPY and PYY-binding protein comprises a CDR (e.g., three CDRs) at least about 80% or 85% or 90% or 95% or 97% or 98% or 99% identical to CDR(s) of a V_{H} or V_{L} as described herein according to any example, wherein the protein is capable of specifically binding to NPY and PYY

For example, and as discussed herein, it is also known in the art that the five C-terminal residues of heavy chain CDR2 can be mutated to conservative or non-conservative amino acid substitutions (31% of residues) (Padlan et al., FASEB J. 9: 133-139, 1995). Thus, a protein can comprise a CDR2 having at least about 69% identity to a heavy chain CDR2 sequence disclosed herein.

In another example, a nucleic acid of the disclosure comprises a sequence at least about 80% or 85% or 90% or 95% or 97% or 98% or 99% identical to a sequence set forth herein and encoding a NPY and PYY-binding protein which is capable of specifically binding to NPY and PYY, which differs from a sequence exemplified herein as a result of degeneracy of the genetic code.

The % identity of a nucleic acid or polypeptide is determined by GAP (Needleman and Wunsch. Mol. Biol. 48, 443-453, 1970) analysis (GCG program) with a gap creation penalty=5, and a gap extension penalty=0.3. The query sequence is at least 50 residues in length, and the GAP analysis aligns the two sequences over a region of at least 50 residues. For example, the query sequence is at least 100 residues in length and the GAP analysis aligns the two sequences over a region of at least 100 residues. For example, the two sequences are aligned over their entire length.

The present disclosure also contemplates a nucleic acid that hybridizes under stringent hybridization conditions to a nucleic acid encoding a NPY and PYY-binding protein described herein. A “moderate stringency” is defined herein as being a hybridization and/or washing carried out in 2×SSC buffer, 0.1% (w/v) SDS at a temperature in the range 45° C. to 65° C., or equivalent conditions. A “high stringency” is defined herein as being a hybridization and/or wash carried out in 0.1×SSC buffer, 0.1% (w/v) SDS, or lower salt concentration, and at a temperature of at least 65° C., or equivalent conditions. Reference herein to a particular level of stringency encompasses equivalent conditions using wash/hybridization solutions other than SSC known to those skilled in the art. For example, methods for calculating the temperature at which the strands of a double stranded nucleic acid will dissociate (also known as melting temperature, or Tm) are known in the art. A temperature that is similar to (e.g., within 5° C. or within 10° C.) or equal to the Tm of a nucleic acid is considered to be high stringency. Medium stringency is to be considered to be within 10° C. to 20° C. or 10° C. to 15° C. of the calculated Tm of the nucleic acid.

The present disclosure also contemplates mutant forms of a NPY and PYY-binding protein of the disclosure comprising one or more conservative amino acid substitutions compared to a sequence set forth herein. In some examples, the NPY and PYY-binding protein comprises 10 or fewer, e.g., 9 or 8 or 7 or 6 or 5 or 4 or 3 or 2 or 1 conservative amino acid substitutions. A “conservative amino acid substitution” is one in which the amino acid residue is replaced with an amino acid residue having a similar side chain and/or hydropathicity and/or hydrophilicity.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), fi-branched side chains (e.g., threonine, valine, isoleucine) and aromatic side chains (e.g., tyrosine, phenylalanine, tryptophan, histidine). Hydropathic indices are described, for example in Kyte and Doolittle J. Mol. Biol., 157: 105-132, 1982 and hydrophylic indices are described in, e.g., U.S. Pat. No. 4,554,101.

The present disclosure also contemplates non-conservative amino acid changes. For example, of particular interest are substitutions of charged amino acids with another charged amino acid and with neutral or positively charged amino acids. In some examples, the NPY and PYY-binding protein comprises 10 or fewer, e.g., 9 or 8 or 7 or 6 or 5 or 4 or 3 or 2 or 1 non-conservative amino acid substitutions.

In one example, the mutation(s) occur within a FR of an antigen binding domain of a NPY and PYY-binding protein of the disclosure. In another example, the mutation(s) occur within a CDR of a NPY and PYY-binding protein of the disclosure.

Exemplary methods for producing mutant forms of a NPY and PYY-binding protein include:
mutagenesis of DNA (Thie et al., Methods Mol Biol. 525:309-22, 2009) or RNA (Kopsidas et al., Immunol. Lett. 107(2):163-8, 2006; Kopsidas et al. BMC Biotechnology, 7: 18, 2007; and WO1999/058661);
introducing a nucleic acid encoding the polypeptide into a mutator cell, e.g., XL-1Red, XL-mutS and XL-mutS-Kanr bacterial cells (Stratagene);
DNA shuffling, e.g., as disclosed in Stemmer, Nature 370:389-91, 1994; and
site directed mutagenesis, e.g., as described in Dieffenbach (ed) and Dveksler (ed) (In: PCR Primer: A Laboratory Manual, Cold Spring Harbor Laboratories, NY, 1995).

Exemplary methods for determining biological activity of the mutant NPY and PYY-binding proteins of the disclosure will be apparent to the skilled artisan and/or described herein, e.g., antigen binding. For example, methods for determining antigen binding, competitive inhibition of binding, affinity, association, dissociation and therapeutic efficacy are described herein.

### Methods for Producing Proteins

### Recombinant Expression

As discussed herein, a nucleic acid encoding a NPY and PYY-binding protein of the disclosure (and/or polypeptides included in such a NPY and PYY-binding protein) is introduced into an expression construct, such that it is operably linked to a promoter to thereby facilitate its expression. Methods for producing expression constructs, e.g., cloning into expression constructs/vectors are known in the art and/or described in Ausubel et at (In: Current Protocols in Molecular Biology. Wiley Interscience, ISBN 047 150338, 1987), and (Sambrook et at (In: Molecular Cloning: Molecular Cloning: A Laboratory Manual, Cold Spring Harbor Laboratories, New York, Third Edition 2001) and U.S. Pat. No. 7,270,969.

In one example, the NPY and PYY-binding protein of the disclosure is expressed in a bacterial cell. Typical promoters suitable for expression in bacterial cells such as for example a bacterial cell selected from the group comprising E. coli, Staphylococcus sp, Corynebacterium sp., Salmonella sp., Bacillus sp., and Pseudomonas sp., include, but are not limited to a promoter such as lacz, Ipp, a temperature-sensitive (_{L} or (_{R} promoters, T7, T3, SP6 or semi-artificial promoters such as the IPTG-inducible tac promoter or lacUV5 promoter.

In another example, the NPY and PYY-binding protein is expressed in a yeast cell. Typical promoters suitable for expression in yeast cells such as, Pichia pastoris, Saccharomyces cerevisiae and S. pombe, include, but are not limited to promoters from the following genes ADH1, GAL1, GAL4, CUP1, PHO5, nmt, RPR1, or TEF1.

In a further example, the NPY and PYY-binding protein is expressed in an insect cell. Typical promoters suitable for expression in insect cells, or in insects, include, but are not limited to, the OPEI2 promoter, the insect actin promoter isolated from Bombyx muni, the Drosophila sp. dsh promoter (Marsh et al Hum. Mol. Genet. 9, 13-25, 2000).

A NPY and PYY-binding protein of the disclosure can also be expressed in plant cells. Promoters for expressing peptides in plant cells are known in the art, and include, but are not limited to, the Hordeum vulgare amylase gene promoter, the cauliflower mosaic virus 35S promoter, the nopaline synthase (NOS) gene promoter, and the auxin inducible plant promoters P1 and P2.

In one example, a NPY and PYY-binding protein of the disclosure is expressed in a mammalian cell or in a mammal. Typical promoters suitable for expression in a mammalian cell include, for example a promoter selected from the group consisting of, retroviral LTR elements, the SV40 early promoter, the SV40 late promoter, the CMV IE (cytomegalovirus immediate early) promoter, the EF₁ promoter (from human elongation factor 1), the EM7 promoter, the UbC promoter (from human ubiquitin C). Examples of useful mammalian host cell lines include monkey kidney CV1 line transformed by SV40 (COS-7); human embryonic kidney line (HEK-293 cells); baby hamster kidney cells (BHK); Chinese hamster ovary cells (CHO); African green monkey kidney cells (VERO-76); or myeloma cells (e.g., NS/0 or SP2/0 cells).

Other elements of expression constructs/vectors are known in the art and include, for example, enhancers, transcriptional terminators, polyadenylation sequences, nucleic acids encoding selectable or detectable markers and origins of replication.

In one example, an expression construct is a bicistronic expression construct. By “bicistronic” is meant a single nucleic acid molecule that is capable of encoding two distinct polypeptides from different regions of the nucleic acid, for example, a single nucleic acid capable of encoding a V_{H} containing polypeptide and a V_{L} containing polypeptide as distinct polypeptides. Generally, the regions encoding each distinct polypeptide are separated by an internal ribosome entry site (IRES) and the region 5′ of the IRES does not comprise a transcription termination sequence. Exemplary IRESs are described, for example, in US20090247455.

Following production of a suitable expression construct, it is introduced into a suitable cell using any method known in the art. Exemplary methods include microinjection, transfection mediated by DEAE-dextran, transfection mediated by liposomes such as by using lipofectamine (Gibco, Md., USA) and/or cellfectin (Gibco, Md., USA), PEG-mediated DNA uptake, electroporation and microparticle bombardment such as by using DNA-coated tungsten or gold particles (Agracetus Inc., WI, USA) amongst others.

The cells used to produce the NPY and PYY-binding protein of this disclosure are then cultured under conditions known in the art to produce a NPY and PYY-binding protein of the disclosure.

Cell free expression systems are also contemplated by the present disclosure, e.g., the TNT T7 and TNT T3 systems (Promega), the pEXP1-DEST and pEXP2-DEST vectors (Invitrogen).

### Protein Purification

Following production/expression, a NPY and PYY-binding protein of the disclosure is purified using a method known in the art. Such purification provides the protein of the disclosure substantially free of nonspecific protein, acids, lipids, carbohydrates, and the like. In one example, the protein will be in a preparation wherein more than about 90% (e.g. 95%, 98% or 99%) of the protein in the preparation is a NPY and PYY-binding protein of the disclosure.

Standard methods of peptide purification are employed to obtain an isolated or recombinant NPY and PYY-binding protein of the disclosure, including but not limited to various high-pressure (or performance) liquid chromatography (HPLC) and non-HPLC polypeptide isolation protocols, such as size exclusion chromatography, ion exchange chromatography, hydrophobic interaction chromatography, mixed mode chromatography, phase separation methods, electrophoretic separations, precipitation methods, salting in/out methods, immunochromatography, and/or other methods.

In one example, affinity purification is useful for isolating a fusion protein comprising a label. Methods for isolating a protein using affinity chromatography are known in the art and described, for example, in Scopes (In: Protein purification: principles and practice, Third Edition, Springer Verlag, 1994). For example, an antibody or compound that binds to the label (in the case of a polyhistidine tag this may be, for example, nickel-NTA) is immobilized on a solid support. A sample comprising a protein is then contacted to the immobilized antibody or compound for a time and under conditions sufficient for binding to occur. Following washing to remove any unbound or non-specifically bound protein, the protein is eluted.

In the case of a NPY and PYY-binding protein comprising a Fc region of an antibody or an antibody heavy chain constant region, protein A or protein G or modified forms thereof can be used for affinity purification. Protein A is useful for isolating purified proteins comprising a human γ1, γ2, or γ4 heavy chain Fc region. Protein G is recommended for all mouse Fc isotypes and for human γ3.

### Conjugates

In one example, a NPY and PYY-binding protein of the present disclosure is conjugated to a compound. For example, the compound is selected from the group consisting of a radioisotope, a detectable label, a therapeutic compound, a colloid, a toxin, a nucleic acid, a peptide, a protein, a compound that increases the half life of the NPY and PYY-binding protein in a subject and mixtures thereof.

The other compound can be directly or indirectly bound to the NPY and PYY-binding protein (e.g., can comprise a linker in the case of indirect binding). Examples of compounds include, a radioisotope (e.g., iodine-131, yttrium-90 or indium-111), a detectable label (e.g., a fluorophore or a fluorescent nanocrystal), a therapeutic compound (e.g., a chemotherapeutic or an anti-inflammatory), a colloid (e.g., gold), a toxin (e.g., ricin or tetanus toxoid), a nucleic acid, a peptide (e.g., a serum albumin binding peptide), a protein (e.g., a protein comprising an antigen binding domain of an antibody or serum albumin), a compound that increases the half life of the NPY and PYY-binding protein in a subject (e.g., polyethylene glycol or other water soluble polymer having this activity) and mixtures thereof. Exemplary compounds that can be conjugated to a NPY and PYY-binding protein of the disclosure and methods for such conjugation are known in the art and described, for example, in WO2010/059821.

Some exemplary compounds that can be conjugated to a NPY and PYY-binding protein of the present disclosure are listed in Table 1.

TABLE 1
Compounds Useful In Conjugation.

| Group | Detail |
|---|---|
| Radioisotopes | ¹²³I, ¹²⁵I, ¹³⁰I, ¹³³I, ¹³⁵I, ⁴⁷Sc, ⁷²As, ⁷²Sc, ⁹⁰Y, ⁸⁸Y, ⁹⁷Ru, |
| (either directly | ¹⁰⁰Pd, ^{101m}Rh, ^{101m}Rh, ¹¹⁹Sb, ¹²⁸Ba, ¹⁹⁷Hg, ²¹¹At, ²¹²Bi, ¹⁵³Sm, |
| or indirectly) | ¹⁶⁹Eu, ²¹²Pb, ¹⁰⁹Pd, ¹¹¹In, ⁶⁷Gu, ⁶⁸Gu, ⁶⁷Cu, ⁷⁵Br, ⁷⁶Br, ⁷⁷Br, |
| | ^{99m}Tc, ¹¹C, ¹³N, ¹⁵O, ¹⁸F, ¹⁸⁸Rc, ²⁰³Pb, ⁶⁴Cu, |
| | ¹⁰⁵Rh, ¹⁹⁸Au, ¹⁹⁹Ag, ⁶⁸Ga or ¹⁷⁷Lu |
| Half life extenders | Polyethylene glycol |
| | Glycerol |
| | Glucose |
| Fluorescent probes | Phycoerythrin (PE) |
| | Allophycocyanin (APC) |
| | Alexa Fluor 488 |
| | Cy5.5 |
| Biologics | fluorescent proteins such as Renilla luciferase, |
| | GFP immune modulators, such as cytokines |
| | toxins |
| | an immunoglobulin or antibody or antibody |
| | variable region half life extenders such as |
| | albumin or antibody variable regions or |
| | peptides that bind to albumin |
| Chemotherapeutics | Taxol |
| | 5-FU |
| | Doxorubicin |
| | Idarubicin |

### Screening Assays

NPY and PYY-binding proteins comprising antibody binding domains of the present disclosure are readily screened for biological activity, e.g., as described below.

### Binding Assays

One form of assay is an antigen binding assay, e.g., as described in Scopes (In: Protein purification: principles and practice, Third Edition, Springer Verlag, 1994). Such a method generally involves labeling the NPY and PYY-binding protein and contacting it with immobilized antigen, i.e., NPY and PYY or a peptide comprising conserved region thereof. Following washing to remove non-specific bound protein, the amount of label and, as a consequence, bound protein is detected. Of course, the NPY and PYY-binding protein can be immobilized and the antigen(s) labeled. Alternatively the assay can be performed with immobilized NPY and PYY-binding protein and labeled antigen. Alternatively, or additionally, surface plasmon resonance assays can be used.

In one example, a binding assay is performed with peptide comprising an epitope of NPY and PYY. In this way, NPY and PYY-binding proteins that bind to a specific region of NPY and PYY are selected.

Such an assay is also readily adapted to identify NPY and PYY-binding proteins that do not detectably or significantly bind to PP or any other protein or peptide described herein. For example, the protein is contacted with labeled PP and, following washing to remove non-specifically bound PP, the level of labeled protein detected.

### Inhibition of Receptor Binding

Methods for identifying NPY and PYY-binding proteins that inhibit interaction of NPY and PYY and a Y receptor will be apparent to the skilled artisan based on the description herein.

For example, a cell expressing a Y receptor (e.g., Y2 receptor or Y1 receptor or Y4 receptor or Y5 receptor) or a region of the receptor required for NPY/PYY binding is immobilized on a surface and contacted with NPY and/or PYY and with a NPY and PYY-binding protein to be tested (in the case of controls, no test NPY and PYY-binding protein is added). A reduced level of NPY/PYY bound to the cell or receptor in the presence of the NPY and PYY-binding protein compared to in the absence of the NPY and PYY-binding protein indicates that the NPY and PYY-binding protein inhibits binding of NPY/PYY to the receptor. The assay can also be performed with labeled NPY/PYY to assist with detection.

In some examples, various concentrations of the NPY and PYY-binding protein are tested and the concentration at which 50% of the maximum inhibition of binding of NPY and/or PYY to a receptor by the NPY and PYY-binding protein is determined (this concentration is known as EC₅₀).

In some examples, various concentrations of the NPY and PYY-binding protein are tested and the concentration at which 50% inhibition of binding of NPY and/or PYY to a receptor by the NPY and PYY-binding protein is determined (this concentration is known as IC₅₀).

### Neutralization Assays

Methods for identifying NPY and PYY-binding proteins that neutralize NPY and/or PYY will also be apparent to the skilled artisan, e.g., based on the description herein.

For example, cells expressing a Y receptor through which NPY and/or PYY signal (e.g., Y1 receptor or Y2 receptor or Y4 receptor or Y5 receptor) are contacted with NPY and/or PYY in the presence or absence of a NPY and PYY-binding protein. The level of NPY/PYY-induced signaling in the cells is then detected. As exemplified herein, NPY/PYY signaling can be determined by detecting the amount of phosphorylated ERK. A protein that reduces the amount of phosphorylated ERK in the cells in the presence of NPY/PYY is considered to neutralize NPY and/or PYY signaling.

Additional assays include, for example, contacting Ewing sarcoma cells (e.g., SK-N-MC) in the presence of NPY and/or PYY and a NPY and PYY-binding protein. If NPY/PYY signal through Y1 and/or Y5 receptors in the cells, cell death is induced. Accordingly, a reduction in cell death in the presence of the NPY and PYY-binding protein compared to in the absence of the protein indicates that the protein neutralizes NPY/PYY signaling.

Another assay for assessing NPY/PYY activity involves culturing HEL cells in the presence of a NPY and PYY-binding protein and NPY and/or PYY and assessing intracellular calcium levels. Reduced intracellular calcium levels in the presence of the NPY and PYY-binding protein compared to in the absence of the protein indicates that the protein neutralizes NPY/PYY signaling.

### In Vivo Assays

NPY and PYY-binding proteins of the present disclosure can also be assessed for therapeutic efficacy in an animal model of a condition, e.g., a NPY/PYY-mediated condition. For example, the NPY and PYY-binding protein is administered to a model of cancer, e.g., to a mouse to which cancer cells are administered. Animal survival, tumor growth, tumor metastasis and/or other measures of cancer progression can then be assessed. In this regard, the tumor cells can be administered after the protein for prophylactic studies or before the protein for therapeutic studies.

In another example, an immune response, e.g., T cell response against the cancer cells is assessed, e.g., using an ELISPOT assay.

In another example, a NPY and PYY-binding protein of the disclosure is administered to an animal and its effect on food consumption and/or body mass/weight is assessed. A NPY and PYY-binding protein that increases food consumption and/or body mass/weight is considered to neutralize NPY/PYY signaling.

In a further example, a NPY and PYY-binding protein of the disclosure is administered to a subject, T cells isolated from the subject and stimulated (e.g., with an anti-CD3 antibody) and the proliferation of T cells assessed. As exemplified herein a NPY and PYY-binding protein that increases the amount of T cell proliferation neutralizes NPY/PYY signaling.

### Competitive Binding Assays

Assays for determining a NPY and PYY-binding protein that competitively inhibits binding of an antibody of the disclosure will be apparent to the skilled artisan. For example, the antibody of the disclosure is conjugated to a detectable label, e.g., a fluorescent label or a radioactive label. The labeled antibody and the test NPY and PYY-binding protein are then mixed and contacted with NPY and/or PYY or a peptide comprising an epitope thereof. The level of labeled antibody is then determined and compared to the level determined when the labeled antibody is contacted with the NPY and/or PYY or the peptide comprising an epitope thereof in the absence of the NPY and PYY-binding protein. If the level of labeled antibody is reduced in the presence of the NPY and PYY-binding protein compared to the absence of the NPY and PYY-binding protein, the NPY and PYY-binding protein competitively inhibits binding of the antibody.

Optionally, the NPY and PYY-binding protein is conjugated to a different label than the antibody. This permits detection of the level of binding of the NPY and PYY-binding protein to NPY/PYY or epitope bearing peptide.

In another example, the NPY and PYY-binding protein is permitted to bind to NPY/PYY or a peptide comprising an epitope thereof prior to contacting the NPY/PYY or peptide with an antibody described herein. A reduction in the amount of bound antibody in the presence of the NPY and PYY-binding protein compared to in the absence of the NPY and PYY-binding protein indicates that the NPY and PYY-binding protein competitively inhibits binding of the antibody to NPY/PYY. A reciprocal assay can also be performed using labeled NPY and PYY-binding protein and first allowing the antibody to bind to NPY/PYY or the peptide. In this case, a reduced amount of labeled NPY and PYY-binding protein bound to NPY/PYY or the peptide in the presence of the antibody compared to in the absence of antibody indicates that the NPY and PYY-binding protein competitively inhibits binding of the antibody to NPY/PYY.

### Epitope Mapping Assays

In another example, the epitope bound by a protein described herein is mapped. Epitope mapping methods will be apparent to the skilled artisan. For example, a series of overlapping peptides spanning the NPY/PYY sequence or a region thereof comprising an epitope of interest, e.g., peptides comprising 10-15 amino acids are produced. The NPY and PYY-binding protein is then contacted to each peptide or a combination thereof and the peptide(s) to which it binds determined. This permits determination of peptide(s) comprising the epitope to which the NPY and PYY-binding protein binds.

Alternatively, or in addition, amino acid residues within NPY/PYY are mutated, e.g., by alanine scanning mutagenesis, and mutations that reduce or prevent protein binding are determined. Any mutation that reduces or prevents binding of the NPY and PYY-binding protein is likely to be within the epitope bound by the protein.

A further method involves binding NPY/PYY or a region thereof to an immobilized NPY and PYY-binding protein of the present disclosure and digesting the resulting complex with proteases. Peptide that remains bound to the immobilized protein are then isolated and analyzed, e.g., using mass spectrometry, to determine their sequence.

A further method involves converting hydrogens in NPY/PYY or a region thereof to deuterium atoms and binding the resulting protein to an immobilized NPY and PYY-binding protein of the present disclosure. The deuterium atoms are then converted back to hydrogen, the NPY/PYY or region thereof isolated, digested with enzymes and analyzed, e.g., using mass spectrometry to identify those regions comprising deuterium, which would have been protected from conversion to hydrogen by the binding of a NPY and PYY-binding protein described herein.

### Affinity Assays

Optionally, the dissociation rate constant (k_{d}) or association rate constant (kₐ) or equilibrium binding constant (K_{D}) of a NPY and PYY-binding protein for NPY/PYY or a peptide comprising an epitope thereof is determined. These constants for a NPY and PYY-binding protein are in one example measured by a radiolabeled or fluorescently-labeled NPY/PYY binding assay. This assay equilibrates the protein with a minimal concentration of labeled NPY/PYY in the presence of a titration series of unlabeled NPY/PYY. Following washing to remove unbound NPY/PYY, the amount of label is determined.

Affinity measurements can be determined by standard methodology for antibody reactions, for example, immunoassays, surface plasmon resonance (SPR) (Rich and Myszka Curr. Opin. Biotechnol 11: 54, 2000; Englebienne Analyst. 123: 1599, 1998), isothermal titration calorimetry (ITC) or other kinetic interaction assays known in the art.

In one example, the constants are measured by using surface plasmon resonance assays, e.g., using BIAcore surface plasmon resonance (BIAcore, Inc., Piscataway, N.J.) with immobilized NPY/PYY or a region thereof (e.g., a peptide described herein). Exemplary SPR methods are described in U.S. Pat. No. 7,229,619.

### Half Life Assays

Some NPY and PYY-binding proteins encompassed by the present disclosure have an improved half-life, e.g., are modified to extend their half-life compared to NPY and PYY-binding proteins that are unmodified. Methods for determining a NPY and PYY-binding protein with an improved half-life will be apparent to the skilled person. For example, the ability of a NPY and PYY-binding protein to bind to a neonatal Fc receptor (FcRn) is assessed. In this regard, increased binding affinity for FcRn increased the serum half-life of the NPY and PYY-binding protein (see for example, Kim et al., Eur J Immunol., 24:2429, 1994).

The half-life of a NPY and PYY-binding protein of the disclosure can also be measured by pharmacokinetic studies, e.g., according to the method described by Kim et al, Eur J of Immunol 24:542, 1994. According to this method radiolabeled NPY and PYY-binding protein is injected intravenously into mice and its plasma concentration is periodically measured as a function of time, for example at 3 minutes to 72 hours after the injection. The clearance curve thus obtained should be biphasic, that is, an alpha phase and beta phase. For the determination of the in vivo half-life of the NPY and PYY-binding protein, the clearance rate in beta-phase is calculated and compared with that of the wild type or unmodified NPY and PYY-binding protein.

### Stability Assays

Stability of a NPY and PYY-binding protein of the disclosure can be assessed by any of a variety of assays. For example, the NPY and PYY-binding protein is exposed to a condition, e.g., heat or acid or stored for a period of time (e.g., 1 month) at room temperature. Aggregation of the NPY and PYY-binding protein can then be assessed by determining turbidity (with an increase in turbidity following exposure to the condition indicating instability), size exclusion chromatography, non-reducing gel electrophoresis or a binding or neutralization study described herein.

### Pharmaceutical Compositions

The NPY and PYY-binding protein of the present disclosure or nucleic acid encoding same or cell expressing same (syn. active ingredient) is useful for parenteral, topical, oral, or local administration, aerosol administration, or transdermal administration, for prophylactic or for therapeutic treatment. Other compositions contemplated by the present disclosure comprise compounds that inhibit Y receptor(s) (e.g., a Y1 receptor and/or a Y2 receptor and/or a Y4 receptor and/or a Y5 receptor) or a combination of compounds one that inhibits NPY and one that inhibits PYY.

Formulation of a NPY and PYY-binding protein or nucleic acid encoding same or cell expressing same or compound to be administered will vary according to the route of administration and formulation (e.g., solution, emulsion, capsule) selected. An appropriate pharmaceutical composition can be prepared in a physiologically acceptable carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. A variety of appropriate aqueous carriers are known to the skilled artisan, including water, buffered water, buffered saline, polyols (e.g., glycerol, propylene glycol, liquid polyethylene glycol), dextrose solution and glycine. Intravenous vehicles can include various additives, preservatives, or fluid, nutrient or electrolyte replenishers (See, generally, Remington's Pharmaceutical Science, 16th Edition, Mack, Ed. 1980). The compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate. The NPY and PYY-binding protein or other compound described herein of this disclosure can be lyophilized for storage and reconstituted in a suitable carrier prior to use according to art-known lyophilization and reconstitution techniques.

The optimum concentration of the active ingredient(s) in the chosen medium can be determined empirically, according to procedures well known to the skilled artisan, and will depend on the ultimate pharmaceutical formulation desired.

The dosage ranges for the administration of the NPY and PYY-binding protein or other compound of the disclosure are those large enough to produce the desired effect. For example, the composition comprises a therapeutically or prophylactically effective amount of the NPY and PYY-binding protein or nucleic acid encoding same or cell expressing same.

As used herein, the term “effective amount” shall be taken to mean a sufficient quantity of the NPY and PYY-binding protein, nucleic acid or cells or other compound to induce/increase or inhibit/reduce/prevent signaling of NPY and PYY in a subject. The skilled artisan will be aware that such an amount will vary depending on, for example, the compound/protein and/or the particular subject and/or the type or severity of a condition being treated. Accordingly, this term is not to be construed to limit the disclosure to a specific quantity of compound/protein.

As used herein, the term “therapeutically effective amount” shall be taken to mean a sufficient quantity of NPY and PYY-binding protein, nucleic acid or cells or other compound to reduce or inhibit one or more symptoms of a condition, e.g., cancer or to induce or stimulate an immune response against cancer cells.

As used herein, the term “prophylactically effective amount” shall be taken to mean a sufficient quantity of NPY and PYY-binding protein, nucleic acid or cells or other compound to prevent or inhibit or delay the onset of one or more detectable symptoms of a condition, e.g. cancer and/or to prevent recurrence or metastasis of cancer.

The dosage should not be so large as to cause adverse side effects, such as hyper viscosity syndromes, pulmonary edema, congestive heart failure, and the like. Generally, the dosage will vary with the age, condition, sex and extent of the disease in the patient and can be determined by one of skill in the art. The dosage can be adjusted by the individual physician in the event of any complication. Dosage can vary from about 0.1 mg/kg to about 300 mg/kg, e.g., from about 0.2 mg/kg to about 200 mg/kg, such as, from about 0.5 mg/kg to about 20 mg/kg, in one or more dose administrations daily, for one or several days.

In one example, the NPY and PYY-binding protein is administered at a dosage of between about 1 mg/kg to about 50 mg/kg. In one example, the NPY and PYY-binding protein is administered at a dosage of between about 5 mg/kg to about 30 mg/kg. In one example, the NPY and PYY-binding protein is administered subcutaneously or intravenously.

In some examples, the NPY and PYY-binding protein or other compound is administered at an initial (or loading) dose which is higher than subsequent (maintenance doses). For example, the NPY and PYY-binding protein or other compound is administered at an initial dose of between about 10 mg/kg to about 50 mg/kg. The NPY and PYY-binding protein or other compound is then administered at a maintenance dose of between about 1 mg/kg to about 10 mg/kg. The maintenance doses may be administered every 7-35 days, such as, every 14 or 21 or 28 days.

In some examples, a dose escalation regime is used, in which a NPY and PYY-binding protein or other compound is initially administered at a lower dose than used in subsequent doses. This dosage regime is useful in the case of subject's initially suffering adverse events

In the case of a subject that is not adequately responding to treatment, multiple doses in a week may be administered. Alternatively, or in addition, increasing doses may be administered.

One or more NPY and PYY-binding proteins of the present disclosure can be administered to an individual by an appropriate route, either alone or in combination with (before, simultaneous with, or after) another drug or agent. For example, the NPY and PYY-binding protein of the present disclosure can also be used in combination with a chemotherapy compound, such as caboplatin, cisplatin, cyclophosphamide, docetaxal, doxorubicin, erlotinib, etoposide, fluorouracil, irinotecan, methotrexate, paclitaxel, topotecan, vincristine or vinblastine. In one example, the chemotherapy compound is selected from the group consisting of methotrexate, 1-asparaginase, vincristine, doxorubicin, danorubicin, cytarabine, idarubicin, mitoxantrone, cyclophosphamide, fludarabine, chlorambucil and combinations thereof.

In one example, the NPY and PYY-binding protein of the present disclosure can also be used in combination with a biologic useful for treating a cancer, e.g., rituximab, trastuzumab, bevacizumab, alemtuzumab, panitumumab, or cetuximab.

In a further example, the NPY and PYY-binding protein of the present disclosure can also be used in combination with radiation therapy.

### Methods of Treating Cancer

### Types of Cancer

As discussed herein, the present disclosure provides a method for treating cancer.

Examples of cancer include, but are not limited to, an adenocarcinoma, a squamous cell carcinoma, a digestive/gastrointestinal cancer, an endocrine cancer, an eye cancer, a musculoskeletal cancer, a breast cancer, a neurologic cancer, a genitourinary cancer, a germ cell cancer, a head and neck cancer, a hematologic/blood cancer, a respiratory cancer, a skin cancer, an AIDS-related malignancy or a gynelogic cancer.

An adenocarcinoma is a cancer of an epithelium that originates in glandular tissue. Exemplary adenocarcinomas include forms of colorectal cancer, lung cancer, cervical cancer, prostate cancer, urachus cancer, vulval cancer, breast cancer, esophageal cancer, pancreatic cancer and gastric cancer.

Digestive/gastrointestinal cancers include anal cancer; bile duct cancer; extrahepatic bile duct cancer; appendix cancer; carcinoid tumor, gastrointestinal cancer; colon cancer; colorectal cancer including childhood colorectal cancer; esophageal cancer including childhood esophageal cancer; gallbladder cancer; gastric (stomach) cancer including childhood gastric (stomach) cancer; hepatocellular (liver) cancer including childhood hepatocellular (liver) cancer; pancreatic cancer including childhood pancreatic cancer; sarcoma, rhabdomyosarcoma; rectal cancer; and small intestine cancer.

Endocrine cancers include islet cell carcinoma (endocrine pancreas); adrenocortical carcinoma including childhood adrenocortical carcinoma; gastrointestinal carcinoid tumor; parathyroid cancer; pheochromocytoma; pituitary tumor; thyroid cancer including childhood thyroid cancer; childhood multiple endocrine neoplasia syndrome; and childhood carcinoid tumor.

Eye cancers include intraocular melanoma; and retinoblastoma.

Musculoskeletal cancers include Ewing's family of tumors; osteosarcoma/malignant fibrous histiocytoma of the bone; rhabdomyosarcoma including childhood rhabdomyosarcoma; soft tissue sarcoma including childhood soft tissue sarcoma; clear cell sarcoma of tendon sheaths; and uterine sarcoma.

Neurologic cancers include childhood brain stem glioma; brain tumor; childhood cerebellar astrocytoma; childhood cerebral astrocytoma/malignant glioma; childhood ependymoma; childhood medulloblastoma; childhood pineal and supratentorial primitive neuroectodermal tumors; childhood visual pathway and hypothalamic glioma; other childhood brain cancers; adrenocortical carcinoma; central nervous system lymphoma, primary; childhood cerebellar astrocytoma; neuroblastoma; craniopharyngioma; spinal cord tumors; central nervous system atypical teratoid/rhabdoid tumor; central nervous system embryonal tumors; and supratentorial primitive neuroectodermal tumors including childhood and pituitary tumor.

Genitourinary cancers include bladder cancer including childhood bladder cancer; renal cell (kidney) cancer; ovarian cancer including childhood ovarian cancer; ovarian epithelial cancer; ovarian low malignant potential tumor; penile cancer; prostate cancer; renal cell cancer including childhood renal cell cancer; renal pelvis and ureter, transitional cell cancer; testicular cancer; urethral cancer; vaginal cancer; vulvar cancer; cervical cancer; Wilms tumor and other childhood kidney tumors; endometrial cancer; and gestational trophoblastic tumor.

Germ cell cancers include childhood extracranial germ cell tumor; extragonadal germ cell tumor; ovarian germ cell tumor; and testicular cancer.

Head and neck cancers include lip and oral cavity cancer; childhood oral cancer; hypopharyngeal cancer; laryngeal cancer including childhood laryngeal cancer; metastatic squamous neck cancer with occult primary; mouth cancer; nasal cavity and paranasal sinus cancer; nasopharyngeal cancer including childhood nasopharyngeal cancer; oropharyngeal cancer; parathyroid cancer; pharyngeal cancer; salivary gland cancer including childhood salivary gland cancer; throat cancer; and thyroid cancer.

Hematologic/blood cell cancers include leukemia (e.g., acute lymphoblastic leukemia in adults and children; acute myeloid leukemia, e.g., in adults and children; chronic lymphocytic leukemia; chronic myelogenous leukemia; and hairy cell leukemia); a lymphoma (e.g., AIDS-related lymphoma; cutaneous T-cell lymphoma; Hodgkin's lymphoma including Hodgkin's lymphoma in adults and children; Hodgkin's lymphoma during pregnancy; non-Hodgkin's lymphoma including non-Hodgkin's lymphoma in adults and children; non-Hodgkin's lymphoma during pregnancy; mycosis fungoides; Sezary syndrome; Waldenstrom's macroglobulinemia; and primary central nervous system lymphoma); and other hematologic cancers (e.g., chronic myeloproliferative disorders; multiple myeloma/plasma cell neoplasm; myelodysplastic syndromes; and myelodysplastic/myeloproliferative disorders).

Respiratory cancers include non-small cell lung cancer; small cell lung cancer; malignant mesothelioma including malignant mesothelioma in adults and children; malignant thymoma; childhood thymoma; thymic carcinoma; bronchial adenomas/carcinoids including childhood bronchial adenomas/carcinoids; pleuropulmonary blastoma.

Skin cancers include Kaposi's sarcoma; Merkel cell carcinoma; melanoma; basal cell carcinoma and childhood skin cancer.

In one example, the cancer expresses a Y receptor, such as, a Y1 receptor and/or a Y2 receptor and/or a Y4 receptor and/or a Y5 receptor.

In one example, the cancer does not express a Y1 receptor and/or a Y2 receptor and/or a Y4 receptor and/or a Y5 receptor.

In one example, the cancer does not proliferate or grow in response to PYY and/or NPY.

In one example, the cancer is melanoma.

In one example, the cancer is lung cancer.

In one example, the cancer is breast cancer.

### Compounds Useful for Treating Cancer

As discussed herein, the present disclosure provides methods of treating cancer comprising inhibiting NPY and PYY. Various compounds are useful in such a method, including:
A NPY and PYY binding protein of the present disclosure;
A multi-specific NPY and PYY-binding protein of the present disclosure;
A combination comprising an anti-NPY antibody and an anti-PYY antibody, examples of which are described herein;
A combination comprising at least two of an anti-Y1 receptor antibody, an anti-Y2 receptor antibody, an anti-Y4 receptor antibody and an anti-Y5 receptor antibody;
A combination comprising an anti-Y1 receptor antibody and an anti-Y2 receptor antibody;
A combination comprising at least two of a Y1-receptor antagonist, a Y2 receptor antagonist a Y4 receptor antagonist and a Y5 receptor antagonist; and
A combination comprising a Y1-receptor antagonist and a Y2 receptor antagonist.

Exemplary Y1-receptor antagonists include BVD-10 (CAS#262418-00-8), GR-231,118 (CAS#158859-98-4), BIBO-3304 (CAS#191868-14-1), BIBP-3226 (CAS#159013-54-4) and PD-160,170 (CAS#181468-88-2).

Exemplary 2-receptor antagonists include BIIE-0246 (CAS#246146-55-4), JNJ 5207787 (CAS#683746-68-1), and SF 11 (CAS#443292-81-7).

Exemplary Y5-receptor antagonists include CGP-71683 (CAS#192322-50-2), FMS-586, L-152,804 (CAS#6508-43-6), Lu AA-33810, MK-0557 and NTNCB (CAS#486453-65-0).

Exemplary Y1 and Y2 receptor antagonists are described, for example, in Daniels et al., Proc Natl Acad Sci USA. 92: 9067-9071, 1995.

### Methods of Treating Non-Cancerous Conditions

The present disclosure also contemplates treating non-cancerous conditions. For example, the disclosure contemplates treating a NPY and/or PYY-mediated condition, such as, anorexia or a wasting condition (which can be associated with cancer).

In one example, the condition is a wasting condition, such as cachexia. In one example, the wasting condition is associated with a condition, such as, cancer, metabolic acidosis, infectious diseases, diabetes, autoimmune immune deficiency syndrome (AIDS), autoimmune disorders, addiction to drugs, cirrhosis of the liver, chronic inflammatory disorders, anorexia, chronic heart failure, chronic kidney disease, osteoporosis, skeletal muscle disease, motor neuron disease, multiple sclerosis, muscle atrophy and neurodegenerative disease.

In one example, the wasting condition is cachexia or sarcopenia (e.g., wasting associated with aging).

In one example, the cachexia is associated with cancer, infectious disease (e.g., tuberculosis or leprosy), AIDS, autoimmune disease (including rheumatoid arthritis or type 1 diabetes), cystic fibrosis, drug addiction, alcoholism or liver cirrhosis.

In one exemplary form of the present disclosure the wasting disorder is cachexia associated with cancer. Exemplary cancers are described supra.

In one example, the method additionally comprises identifying a subject suffering from cachexia. Such a subject can be identified, for example, based on detection of unintentional weight loss following diagnosis of another condition (e.g., cancer). For example, the subject can lose at least 5% of their body weight following diagnosis of another condition (e.g., cancer) or within the previous 30 days.

In one example, the method additionally comprises monitoring the weight of the subject and if their weight decreases or does not stabilize or increase administering a further dose of the compound(s).

### NPY Receptor Detection Assays

The following assays can be performed with an antibody against a Y receptor, e.g., a Y1 receptor or a Y2 receptor or a Y4 receptor or a Y5 receptor, e.g., an antibody conjugated to a detectable label. Detection of the Y receptor(s) with an assay described herein is useful for identifying a subject suitable for treatment by performing a method described herein.

An immunoassay is an exemplary assay format for diagnosing a condition in a subject or detecting a Y receptor in a sample. The present disclosure contemplates any form of immunoassay, including Western blotting, enzyme-linked immunosorbent assay (ELISA), fluorescence-linked immunosorbent assay (FLISA), competition assay, radioimmunoassay, lateral flow immunoassay, flow-through immunoassay, electrochemiluminescent assay, nephelometric-based assays, turbidometric-based assay, and fluorescence activated cell sorting (FACS)-based assays.

One form of a suitable immunoassay is, for example, an ELISA or FLISA.

In one form such an assay involves immobilizing an anti-Y receptor antibody onto a solid matrix, such as, for example a polystyrene or polycarbonate microwell or dipstick, a membrane, or a glass support (e.g. a glass slide). A test sample is then brought into direct contact with the antibody and Y receptor bearing cells in the sample are bound or captured. Following washing to remove any unbound cells in the sample, a protein that binds to the Y receptor or cell at a distinct epitope is brought into direct contact with the captured cell. This detector protein is generally labeled with a detectable reporter molecule, such as for example, an enzyme (e.g. horseradish peroxidase (HRP)), alkaline phosphatase (AP) or β-galactosidase) in the case of an ELISA or a fluorophore in the case of a FLISA. Alternatively, a second labeled protein can be used that binds to the detector protein. Following washing to remove any unbound protein the detectable reporter molecule is detected by the addition of a substrate in the case of an ELISA, such as for example hydrogen peroxide, TMB, or toluidine, or 5-bromo-4-chloro-3-indol-beta-D-galaotopyranoside (x-gal). Of course, the immobilized (capture) protein and the detector protein may be used in the opposite manner.

The level of the antigen in the sample is then determined using a standard curve that has been produced using known quantities of the marker or by comparison to a control sample.

The assays described above are readily modified to use chemiluminescence or electrochemiluminescence as the basis for detection.

As will be apparent to the skilled artisan, other detection methods based on an immunosorbent assay are useful in the performance of the present disclosure. For example, an immunosorbent method based on the description supra using a radiolabel for detection, or a gold label (e.g. colloidal gold) for detection, or a liposome, for example, encapsulating NAD+ for detection or an acridinium linked immunosorbent assay.

In another example, a tumor sample is assessed by immunofluorescence or immunohistochemistry for Y receptor expression.

### Kits

The present disclosure additionally comprises a kit comprising one or more of the following:
(i) a NPY and PYY-binding protein of the disclosure or expression construct(s) encoding same;
(ii) a multi-specific NPY and PYY-binding protein of the present disclosure;
(iii) an anti-NPY antibody and an anti-PYY antibody, examples of which are described herein;
(iv) an anti-Y1 receptor antibody, an anti-Y2 receptor antibody and an anti-Y5 receptor antibody;
(v) an anti-Y1 receptor antibody and an anti-Y2 receptor antibody;
(vi) a Y1-receptor antagonist, a Y2 receptor antagonist and a Y5 receptor antagonist; and
(vii) a Y1-receptor antagonist and a Y2 receptor antagonist
(viii) a cell of the disclosure; or
(ix) a pharmaceutical composition of the disclosure.

In the case of a kit for therapeutic/prophylactic use, the kit can additionally comprise a pharmaceutically acceptable carrier.

Optionally a kit of the disclosure is packaged with instructions for use in a method described herein according to any example.

The present disclosure includes the following non-limiting Examples.

### EXAMPLES

### Example 1

### Role of NPY/PYY in Cancer

### 1.1 Methods

### Tumor Cells

Tumor cells used in this study were Lewis Lung Carcinoma (LL2) and B16F10 melanoma. LL2 and B16F10 were cultured in DMEM and RPMI respectively and supplemented with Fetal Calf Serum (FCS), penicillin, glutamine and β-mercaptoethanol. The cells were prepared for injection by culturing to 70% confluence and then half of the media was refreshed and allowed to grow overnight before harvesting. The cells were harvested by washing twice with cold PBS and then injected into the mice in a total volume of 200 μl PBS subcutaneously.

### Antibodies

In order to deplete T cells from, mice received intraperitoneal injection of 200 μl PBS containing 50 μg rat-anti mouse CD4 antibody (clone GK1.5) and 50 μg of rat-anti mouse CD8 antibody (clone 53-6.7) three days and one day before tumour cell challenge followed by two injections per week for the duration of the experiment.

### Animals

NPY^{-/-}, PYY^{-/-} and NPY^{-/-}PYY^{-/-} mice are genetically deleted for the indicated gene(s). C57BL/6 mice were used for the in vivo tumour studies using the anti-NPY/PYY antibody 5E12.

### Tumor Model

Tumor cells were harvested using trypsin and EDTA. Cells were washed twice in ice cold PBS, resuspended in 200 μl PBS and injected subcutaneously in the flank of the mice. Tumor growth was measured in two dimensions using a vernier calliper.

### 1.2 Results

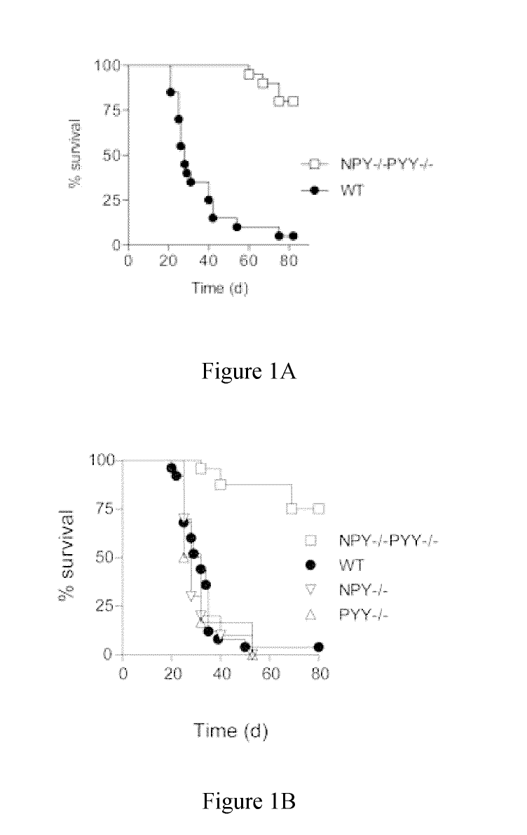

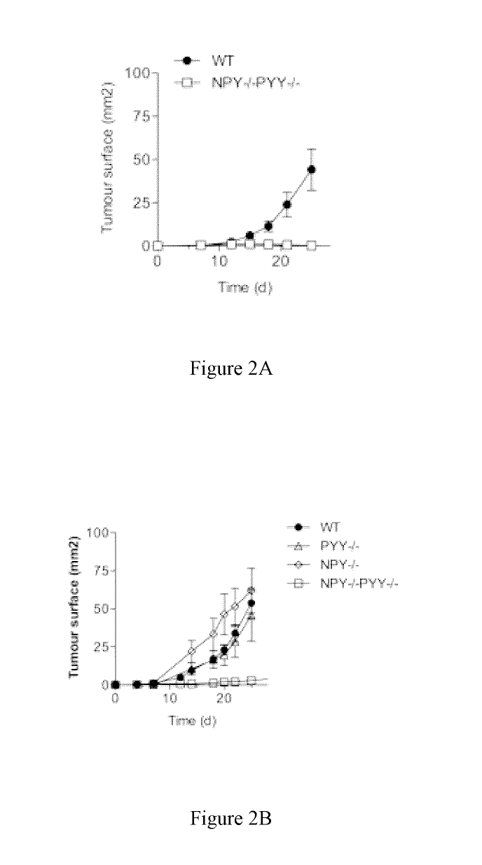

As shown in FIGS. 1A and 1B, mice deficient in NPY and PYY (NPY^{-/-}PYY^{-/-}) spontaneously reject tumors when challenged with a lethal dose of B16F10 melanoma cells or LL2 Lewis lung carcinoma cells. FIGS. 2A and 2B also demonstrate that mice deficient in NPY and PYY (NPY^{-/-}PYY^{-/-}) either do not develop tumors or that any tumors that develop are smaller than in wild-type mice. FIGS. 1B and 2B also show that inhibition of expression of NPY or PYY alone is not sufficient to prevent tumor growth or to significantly increase survival of mice administered lung carcinoma cells. These data indicate that inhibition of both NPY and PYY is important in the treatment of cancer.

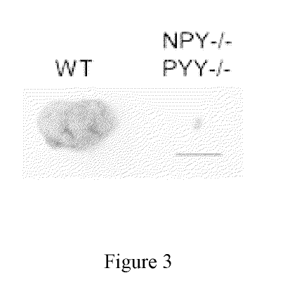

Tumor size in NPY^{-/-}PYY^{-/-} mice administered with melanoma cells were considerably smaller than in wild type mice (FIG. 3).

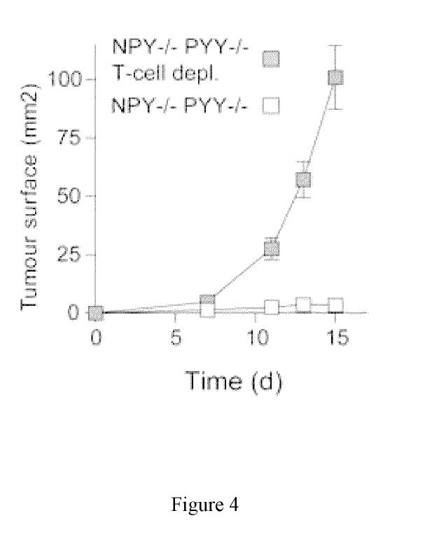

Additional experiments conducted to characterize the anti-cancer effect of inhibiting NPY/PYY signaling involved depleting T cells from NPY^{-/-}PYY^{-/-} mice administered B16 melanoma cells. As shown in FIG. 4, mice depleted in T cells were unable to suppress tumor growth unlike NPY^{-/-}PYY^{-/-} mice having T cells. Accordingly, an effect of inhibiting NPY/PYY signaling appears to be inducing or enhancing an anti-cancer immune response.

### Example 2

### Production and Characterization of an Anti-NPY and PYY Antibody

### 2.1 Methods

### Overview

Generation of an antibody that bound and neutralized both NPY and PYY in mouse and humans presented several challenges.

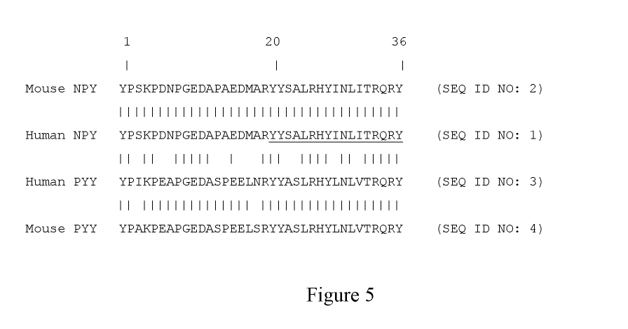

Firstly, although human NPY and PYY are highly homologous (i.e. 67% identical) the longest stretch of identity between these 36 amino acid peptides is only 5 residues (see FIG. 5). While these differences are spread fairly evenly throughout the sequence, 8 differences occur in the N-terminal half of the peptide, with only 4 differences in the C-terminal half. To increase chances of generating an antibody that cross-reacted with both NPY and PYY a peptide for immunization was designed comprising C-terminal residues 20-36 of NPY (underlined in FIG. 5). To overcome the high level of sequence identity between human and mouse peptides, which hinders immunizations, NPY^{-/-}PYY^{-/-} double knockout mice were utilized. Thus, human and mouse NPY are identical, with human and mouse PYY almost identical, differing in just 2 residues of 36 positions (94% identity).

Secondly, human and mouse NPY are identical. Furthermore human and mouse PYY are almost identical, differing in just 2 residues of 36 positions (94% identity). Because the immune system has mechanisms to delete self-reactive antibody secreting B cells, NPY^{-/-} or PYY^{-/-} single gene knockout mouse or the NPY^{-/-}PYY^{-/-} double knockout mouse was used to increase the chances of generating an antibody to human NPY/PYY.

### Production of Immunizing Peptide

In common with many other biologically active peptides, NPY is formed from a precursor that is enzymatically cleaved and has an amidated C-terminal amino acid. Therefore the peptide synthesized for these studies comprised the sequence:
_{Nterminus}-yysalrhyinlitrqry-amide-_{C-TerMINus} (SEQ ID NO: 78)
   with an NH₂ group on its C-terminus. This peptide, designated NPY_{20-36NH2} (or HBY or immunizing peptide) was synthesized using standard techniques at Mimotopes (Melbourne, Australia). It was conjugated to KLH via gluteraldehyde. The peptide was freeze-dried, then resuspended in sterile water at 448 μM (1 mg/mL) prior to immunization.

### Injection and Monitoring of Mice

NPY^{-/-}PYY^{-/-} double-knockout mice were immunized with the KLH conjugated HBY peptide (HBY-KLH, Sequence of HBY: SEQ ID NO: 78) using the regime summarized in Table 2. Peptide was prepared for immunization by diluting the HBY-KLH peptide to either 500 μg/mL (prime) or 250 μg/mL (boosts) in sterile PBS and either 50% v/v Imject Freund's Complete Adjuvant (FCA) or 50% v/v Imject Freund's Incomplete Adjuvant (FIA)—(Thermo Scientific). Mice were immunized intra-peritoneally. For the final boost, HBY-KLH peptide was diluted in sterile PBS only. The peptide preparation was injected i.v. into mice via one of the tail veins.

TABLE 2
Immunization Regime For Raising Anti-NPY/PYY
Immune Response In Five NPY^{-/-}PYY^{-/-} Double-Knockout Mice

| Day | Immunization |
|---|---|
| 0 | 50 μg HBY-KLH injected i.p. in 200 μL FCA |
| 14 | 25 μg HBY-KLH injected i.p. in 200 μL FIA |
| 28 | 25 μg HBY-KLH injected i.p. in 200 μL FIA |
| 49 | 25 μg HBY-KLH injected i.p. in 200 μL FIA |
| 74 | 50 μg HBY-KLH injected i.v. in 100 μL PBS |

### Analysis of Serum Titre in Immunized Mice

Blood samples were collected from immunized mice one week after third boost immunization. HBY-specific serum titre of immunized mice was measured by ELISA.

### Generation of and Screening of Hybridomas

All cell cultures described in the section were incubated at 37° C. with 5% v/v CO₂. Four days after the final i.v. immunization with HBY-KLH, the spleen was removed from an immunized mouse and a cell suspension generated. The spleen cells were then fused to SP2/0 myeloma cells using standard PEG fusion methods. Hybridomas were then selected in HAT selection medium.

ELISA was then used to select hybridomas secreting antibody that bound to the HBY peptide.

Any hybridomas secreting antibodies that bound to HBY peptide were expanded. Expanded wells with ongoing ELISA reactivity to HBY peptide were selected for subcloning. Cells were then diluted and subcloned and again screened for secretion of antibodies reactive with HBY peptide.

### Antibody Characterization

Isotyping

The isotype of antibodies was determined by two ELISAs, one which used HBY peptide as a capture reagent and the other that used goat anti-mouse Ig as a capture reagent. Following capture of antibody from hybridoma supernatant, plates were washed and incubated with HRP-conjugated antibodies against isotypes IgG1, IgG2a, IgG2b, IgG2c, IgG3 and IgM (Southern Biotech) as well as antimouse IgG+M (Jackson). Substrate reaction was stopped after 15 min with 100 μL 1M H₂SO₄ and O.D. 450 nm was measured.

### Sequencing of Heavy and Light Chain Variable Domains

RNA was obtained from hybridomas using RNEASY RNA purification kit (QIAGEN). Briefly, two aliquots of about 4×10⁶ cells were collected into 15 mL centrifuge tubes from passaged hybridoma cells.

cDNA was prepared from purified RNA using a Superscript III kit (Invitrogen), essentially according to manufacturer's instructions.

The V_{H} gene was amplified by PCR with a specific 3′ primer and various sets of 5′ primers. The amplified product was then cloned into electro-competent XL1-Blue E. coli (Invitrogen) using the TOPO vector (Invitrogen), essentially according to manufacturer's instructions. Cloned nucleic acid was then amplified and submitted to the Australian Genome Research Facility (Westmead, Australia) for sequencing.

A hybridoma cell line was also submitted to SydLabs (USA) for sequencing of the V_{H} and V_{L} regions.

### Determining Half-Life of MAb5E12

C57BL/6 mice were injected with 200 μl PBS i.p containing the indicated amounts of antibody. Serum was taken 2 hours, 3 days and 6 days after injection and the serum was used in an ELISA to detect the amount of NPY/PYY specific antibodies.

### Characterization of Affinity and Epitope Mapping for MAb 5E12

Purification of MAb 5E12

Hybridoma line 5E12-B7 was adapted over a two-week period into serum-free medium (CD Hybridoma, GIBCO) supplemented with 2 mM L-glutamine 50 U/mL penicillin and 50 μg/mL streptomycin. The cells in serum-free medium were then expanded to 150 cm² flasks. Cells suspensions were then harvested from the flasks and cells removed by centrifugation. Alternatively, 20 mL of 5E12-B7 serum-free cell suspension was seeded into the antibody collection compartment of a CELLINE flask (BD Biosciences) containing 1 L of CD hybridoma supplemented as above. Cell suspensions were harvested weekly from the antibody collection compartment and centrifuged. Decanted supernate was filtered and stored at 4° C.

Supernate was combined with Protein G Sepharose 4 Fast Flow (GE Healthcare) and incubated on a roller-shaker overnight at 4° C. The mixture was then run through a gravity flow column. The protein G Sepharose beads collected at the bottom of the column were washed with 5 column volumes of tissue culture grade PBS (GIBCO). Ig was eluted from the beads by adding 10×2 mL fractions of 0.1M glycine, 0.1M NaCl (pH 3.0).

### Design of Peptides for Epitope Mapping

Several series of peptides were designed for epitope mapping and determination of mAb 5E12-B7 affinity to the target peptides NPY and PYY (Table 3). Firstly peptides 1p1-1p18 were designed based on the last 25 residues of NPY, and contained single alanine substitutions sequentially at residues 20-36 of peptide NPY. Peptides 1p19-1p32 were 10mer peptides starting at residues 14-23 of NPY (peptide 1p19) and shifting by one residue until the final 10 residues of NPY were reached (peptide 1p32). Peptides 2p1-2p16 were 17mers based on the immunizing peptide (HBY) with a shrinking C-terminus being progressively replaced with a growing poly-alanine N-terminus. Peptide 2p17 was the 17mer immunizing peptide (HBY) with four alanine residues added to the C-terminus. Peptide 2p18 was based on the last 17 residues of mouse PP except that the third last residue (proline) was replaced with the glutamine residue found in NPY and PYY. Peptide 2p19 was identical to the HBY immunizing peptide except that the amide group found at the C-terminus of naturally occurring NPY was replaced with a carboxyl group. Peptide 2p20 was a 17mer containing a non-helical chain of 14 glycine residues followed by the last three residues of the NPY C-terminus. Peptides 3p1-3p6 were designed to present the last 8 residues of the C terminus of peptides mPP, hPP, PYY, NPY, FMRF and NPFF on a poly-alanine helix (up to 8 Ala residues). Peptides 3p10-3p19 were 17mer poly-alanine peptides with the last three residues (-QRY-NH₂) of NPY. In each case, a single residue of the QRY C-terminus was substituted for a similar amino acid residue.

All peptides listed in Table 3 were synthesized by Mimotopes Australia except for the NPY, mPYY, hPYY and hPP whole peptides which were purchased from Phoenix Peptides (USA). All peptides were synthesized with an amide group at the C-terminus unless otherwise specified.

TABLE 1
Peptides Designed For Epitope Mapping And Characterization
Of mAB 5e12-B7 Affinity. Differences From Mouse NPY Peptide
Sequence Are Shown In Bold And Underlined

| | | | | | SEQ |
|---|---|---|---|---|---|
| Peptide | Description | NTerm | Sequence | CTerm | ID NO |
| 1p1 | NPY last 17 residues Alanine Scan | H— | APAEDMARYYSALRHYINLITRQRY | —NH2 | 7 |
| 1p2 | NPY last 17 residues Alanine Scan | H— | APAEDMARAYSALRHYINLITRQRY | —NH2 | 8 |
| 1p3 | NPY last 17 residues Alanine Scan | H— | APAEDMARYASALRHYINLITRQRY | —NH2 | 9 |
| 1p4 | NPY last 17 residues Alanine Scan | H— | APAEDMARYYAALRHYINLITRQRY | —NH2 | 10 |
| 1p5 | NPY last 17 residues Alanine Scan | H— | APAEDMARYYSALRHYINLITRQRY | —NH2 | 11 |
| 1p6 | NPY last 17 residues Alanine Scan | H— | APAEDMARYYSAARHYINLITRQRY | —NH2 | 12 |
| 1p7 | NPY last 17 residues Alanine Scan | H— | APAEDMARYYSALAHYINLITRQRY | —NH2 | 13 |
| 1p8 | NPY last 17 residues Alanine Scan | H— | APAEDMARYYSALRAYINLITRQRY | —NH2 | 14 |
| 1p9 | NPY last 17 residues Alanine Scan | H— | APAEDMARYYSALRHAINLITRQRY | —NH2 | 15 |
| 1p10 | NPY last 17 residues Alanine Scan | H— | APAEDMARYYSALRHYANLITRQRY | —NH2 | 16 |
| 1p11 | NPY last 17 residues Alanine Scan | H— | APAEDMARYYSALRHYIALITRQRY | —NH2 | 17 |
| 1p12 | NPY last 17 residues Alanine Scan | H— | APAEDMARYYSALRHYINAITRQRY | —NH2 | 18 |
| 1p13 | NPY last 17 residues Alanine Scan | H— | APAEDMARYYSALRHYINLATRQRY | —NH2 | 19 |
| 1p14 | NPY last 17 residues Alanine Scan | H— | APAEDMARYYSALRHYINLIARQRY | —NH2 | 20 |
| 1p15 | NPY last 17 residues Alanine Scan | H— | APAEDMARYYSALRHYINLITAQRY | —NH2 | 21 |
| 1p16 | NPY last 17 residues Alanine Scan | H— | APAEDMARYYSALRHYINLITRARY | —NH2 | 22 |
| 1p17 | NPY last 17 residues Alanine Scan | H— | APAEDMARYYSALRHYINLITRQAY | —NH2 | 23 |
| 1p18 | NPY last 17 residues Alanine Scan | H— | APAEDMARYYSALRHYINLITRQRA | —NH2 | 24 |
| 1p19 | 10 mer shifting across NPY | H— | AEDMARYYSA | —NH2 | 25 |
| 1p20 | 10 mer shifting across NPY | H— | EDMARYYSAL | —NH2 | 26 |
| 1p21 | 10 mer shifting across NPY | H— | DMARYYSALR | —NH2 | 27 |
| 1p22 | 10 mer shifting across NPY | H— | MARYYSALRH | —NH2 | 28 |
| 1p23 | 10 mer shifting across NPY | H— | ARYYSALRHY | —NH2 | 29 |
| 1p24 | 10 mer shifting across NPY | H— | RYYSALRHYI | —NH2 | 30 |
| 1p25 | 10 mer shifting across NPY | H— | YYSALRHYIN | —NH2 | 31 |
| 1p26 | 10 mer shifting across NPY | H— | YSALRHYINL | —NH2 | 32 |
| 1p27 | 10 mer shifting across NPY | H— | SALRHYINLI | —NH2 | 33 |
| 1p28 | 10 mer shifting across NPY | H— | ALRHYINLIT | —NH2 | 34 |
| 1p29 | 10 mer shifting across NPY | H— | LRHYINLITR | —NH2 | 35 |
| 1p30 | 10 mer shifting across NPY | H— | RHYINLITRQ | —NH2 | 36 |
| 1p31 | 10 mer shifting across NPY | H— | HYINLITRQR | —NH2 | 37 |
| 1p32 | 10 mer shifting across NPY | H— | YINLITRQRY | —NH2 | 38 |
| 2p1 | Poly Ala with shrinking NPY C term | H— | AYSALRHYINLITRQRY | —NH2 | 39 |
| 2p2 | Poly Ala with shrinking NPY C term | H— | AASALRHYINLITRQRY | —NH2 | 40 |
| 2p3 | Poly Ala with shrinking NPY C term | H— | AAAALRHYINLITRQRY | —NH2 | 41 |
| 2p4 | Poly Ala with shrinking NPY C term | H— | AAAAARHYINLITRQRY | —NH2 | 42 |
| 2p5 | Poly Ala with shrinking NPY C term | H— | AAAAAAHYINLITRQRY | —NH2 | 43 |
| 2p6 | Poly Ala with shrinking NPY C term | H— | AAAAAAAYINLITRQRY | —NH2 | 44 |
| 2p7 | Poly Ala with shrinking NPY C term | H— | AAAAAAAAINLITRQRY | —NH2 | 45 |
| 2p8 | Poly Ala with shrinking NPY C term | H— | AAAAAAAAANLITRQRY | —NH2 | 46 |
| 2p9 | Poly Ala with shrinking NPY C term | H— | AAAAAAAAAALITRQRY | —NH2 | 47 |
| 2p10 | Poly Ala with shrinking NPY C term | H— | AAAAAAAAAAAITRQRY | —NH2 | 48 |
| 2p11 | Poly Ala with shrinking NPY C term | H— | AAAAAAAAAAAATRQRY | —NH2 | 49 |
| 2p12 | Poly Ala with shrinking NPY C term | H— | AAAAAAAAAAAATRQRY | —NH2 | 50 |
| 2p13 | Poly Ala with shrinking NPY C term | H— | AAAAAAAAAAAAARQRY | —NH2 | 51 |
| 2p14 | Poly Ala with shrinking NPY C term | H— | AAAAAAAAAAAAAAQRY | —NH2 | 52 |
| 2p15 | Poly Ala with shrinking NPY C term | H— | AAAAAAAAAAAAAAARY | —NH2 | 53 |
| 2p16 | Poly Ala with shrinking NPY C term | H— | AAAAAAAAAAAAAAAAY | —NH2 | 54 |
| 2p17 | HBY with polyAlanine C terminus | H— | YYSALRHYINLITRQRYAAAA | —NH2 | 55 |
| 2p18 | mPP with last 4 NPY residues | H— | YETQLRRYINTLTRQRY | —NH2 | 56 |
| 2p19 | HBY with OH C term | H— | YYSALRHYINLITRQRY | —OH | 57 |
| 2p20 | Poly Glycine with last 3 NPY | H— | GGGGGGGGGGGGGGQRY | —NH2 | 58 |
| | residues | | | | |
| 3p1 | ala-mPP | H— | AAAAAAAAANTLTRPRY | —NH2 | 59 |
| 3p2 | ala-hPP | H— | AAAAAAAAANMLTRPRY | —NH2 | 60 |
| 3p3 | ala-PYY | H— | AAAAAAAAANLVTRQRY | —NH2 | 61 |
| 3p4 | ala-NPY | H— | AAAAAAAAANLITRQRY | —NH2 | 62 |
| 3p5 | ala-FMRF | H— | AAAAAAAAAAAAAFMRF | —NH2 | 63 |
| 3p6 | ala-NPFF | H— | AAAAAAAAAFLFQPQRF | —NH2 | 64 |
| 3p7 | Brain Neuropeptide I | H— | AGEGLSSPFWSLAAPQRF | —NH2 | 65 |
| 3p8 | Poly alanine with last 4 residues | H— | AAAAAAAAAAAAAPQRF | —NH2 | 66 |
| | NPFF | | | | |
| 3p9 | Poly alanine with proline, last 3 | H— | AAAAAAAAAAAAAPQRY | —NH2 | 67 |
| | residues NPY | | | | |
| 3p10 | NPY C terminus with similar a.a. | H— | AAAAAAAAAAAAAANRY | —NH2 | 68 |
| | substitutions | | | | |
| 3p11 | NPY C terminus with similar a.a. | H— | AAAAAAAAAAAAAAERY | —NH2 | 69 |
| | substitutions | | | | |
| 3p12 | NPY C terminus with similar a.a. | H— | AAAAAAAAAAAAAADRY | —NH2 | 70 |
| | substitutions | | | | |
| 3p13 | NPY C terminus with similar a.a. | H— | AAAAAAAAAAAAAARRY | —NH2 | 71 |
| | substitutions | | | | |
| 3p14 | NPY C terminus with similar a.a. | H— | AAAAAAAAAAAAAAKRY | —NH2 | 72 |
| | substitutions | | | | |
| 3p15 | NPY C terminus with similar a.a. | H— | AAAAAAAAAAAAAAQQY | —NH2 | 73 |
| | substitutions | | | | |
| 3p16 | NPY C terminus with similar a.a. | H— | AAAAAAAAAAAAAAQKY | —NH2 | 74 |
| | substitutions | | | | |
| 3p17 | NPY C terminus with similar a.a. | H— | AAAAAAAAAAAAAAQRH | —NH2 | 75 |
| | substitutions | | | | |
| 3p18 | NPY C terminus with similar a.a. | H— | AAAAAAAAAAAAAAQRF | —NH2 | 76 |
| | substitutions | | | | |
| 3p19 | NPY C terminus with similar a.a. | H— | AAAAAAAAAAAAAAQRW | —NH2 | 77 |
| | substitutions | | | | |
| HBY | mouse NPY last 17 res | H— | YYSALRHYINLITRQRY | —NH2 | 78 |
| mNPY | mouse NPY (phoenix) | H— | YPSKPDNPGEDAPAEDMARYYSALR | —NH2 | 2 |
| | | | HYINLITRQRY | | |
| mPYY | mouse PYY (Phoenix) | H— | YPAKPEAPGEDASPEELSRYYASLR | —NH2 | 4 |
| | | | HYLNLVTRQRY | | |
| hPYY | human PYY (Phoenix) | H— | YPIKPEAPGEDASPEELNRYYASLR | —NH2 | 3 |
| | | | HYLNLVTRQRY | | |
| mPP | mouse PP (custom) | H— | APLEPMYPGDYATPEQMAQYETQLR | —NH2 | 6 |
| | | | RYINTLTRPRY | | |
| hPP | human PP (Phoenix) | H— | APLEPVYPGDNATPEQMAQYAADLR | —NH2 | 5 |
| | | | RYINMLTRPRY | | |

### Epitope Mapping of MAb 5E12 by ELISA

ELISA was used to map the reactivity of purified MAb 5E12-B7 against the peptides described in Table 3. Briefly, ELISA microwell plates (96-well Maxisorp, Nunc) were coated overnight at 4° C. with 50 μL of mapping peptides (Table 3) diluted to 20 μg/mL in carbonate coating buffer (CCB). Between each of the following steps, microwell plates were washed three times with PBST using a plate washer (BioTek). Coated wells were blocked with 200 μL PBS containing 5% w/v skim milk (Diploma). Blocked wells were incubated for 1 hr with 50 μL of MAb 5E12-B7 diluted to 1 μg/mL in skim milk/PBST. Wells were then incubated for 1 hr with 50 μL Peroxidase-conjugated AffiniPure F(ab)2 fragment goat anti-mouse secondary antibody (Jackson) diluted 1:5000 in skim milk/PBST. Ig binding was detected by incubation of wells with 50 μL TMB substrate (Becton Dickinson). Substrate reaction was stopped after 15 min with 50 μL 1M H₂SO₄ and O.D. 450 nm was measured.

### Determination of MAb Affinity to NPY, PYY and PP Peptides by Octet

Purified MAb 5E12-B7 was biotinylated using EZ-Link® NHS-PEO₄-Biotinylation reagent (Thermo Scientific), essentially in accordance with manufacturer's instructions.

Affinity of MAb to peptides NPY, mPYY, hPYY, mPP and hPP were measured using an OCTET RED96 system with Octet SA sensors (ForteBio). Briefly, black 96-well plates were prepared with columns containing 200 μL/well of all reagents required for determination of affinity. In general, Octet SA sensors were first placed in wells containing Octet running buffer for 60 seconds to establish a baseline. Sensors were then transferred to wells containing 50 μg/mL biotinylated 5E12-B7 for 480 seconds to load the SA coated tips. Sensors were then regenerated by 3×5 second incubations in Regeneration Buffer (10 mM Glycine, pH 2) followed each time by a 5 second incubation in Octet running buffer. Sensors were then placed in fresh running buffer for 60 seconds to establish a baseline. Association was then measured by incubation the sensors for 10 min in 8 wells containing a serial dilution of peptide (0, 0.33 to 243 nM for NPY and PYY peptides, 0, 9 to 6560 nM for PP peptides). The sensors were then transferred to wells containing Octet running buffer for 15 min to measure dissociation of the peptides. The regeneration step was repeated before measurement of association/dissociation with a different peptide. All peptide measurements were performed in duplicate. The k_{d}, kₐ and K_{D} were determined for each peptide using the accompanying Octet software (Bioforte).

### Epitope Mapping and Determination of MAb Affinity by Biacore

Epitope mapping and determination of the affinity of MAb 5E12-B7 was also performed using a Biacore 2000 unit (GE Healthcare). Briefly, purified MAb 5E12-B7 was biotinylated as described above, except that the biotinylation reaction was performed at a 20:1 molar ratio of biotin:Ig. An SA-Chip (GE Healthcare) was docked into the Biacore 2000 unit and HBS-EP buffer (GE) was allowed to run over all 4 flow channels at 40 μL/min until signal had stabilized. The flow path was then switched to channel 4 only and HBS-EP containing 60 μg/mL biotinylated antibody was injected at 40 μL/min for 5 min to couple MAb 5E12-B7 to the chip via biotin-streptavidin binding. HBS-EP buffer was then run over channel 4 at 40 μL/min for over 30 min to remove all uncoupled Ig from the Biacore system then the flow path was changed to pass through all 4 flow channels. Association and dissociation of the peptides in Table 3 was measured using the Biacore Application Wizard. In each case, a baseline was established by passing HBS-EP over all 4 channels at 40 μL/min for 1 min before association was measure by injection of peptide (diluted to 1, 10, 100 or 1000 nM in HBS-EP) at 40 μL/min for 5 min. Dissociation was measured by allowing HBS-EP to pass over all 4 channels at 40 μL/min for 30 min. The k_{d}, kₐ and K_{D} were determined for each peptide by curve fitting using the BIAevalutation software.

### Results

### Generation and Characterization of Antibody 5E12

NPY^{-/-}PYY^{-/-} mice were immunized with KLH-conjugated peptide HBY (NPY_{20-36NH2}) using the regime in Table 2. After the fourth immunization, serum from an immunized mouse showed immunoreactivity with the immunizing peptide. This mouse was then selected for hybridoma generation and hybridomas screened for immunoreactivity with immunizing peptide, mouse NPY, human PYY and 0.1% BSA. Positive hybridomas were then expanded and subcloned, and subclones of one hybridoma (5E12) having immunoreactivity to the immunizing peptide selected. Subclone B7 (i.e., 5E12-B7) was selected.

Isotyping of Ig was performed on 5E12 and subclone 5E12-B7 by sandwich ELISA. Analysis of HBY binding antibodies indicated that the antibody that binds to NPY and PYY is Ig2a/2c.

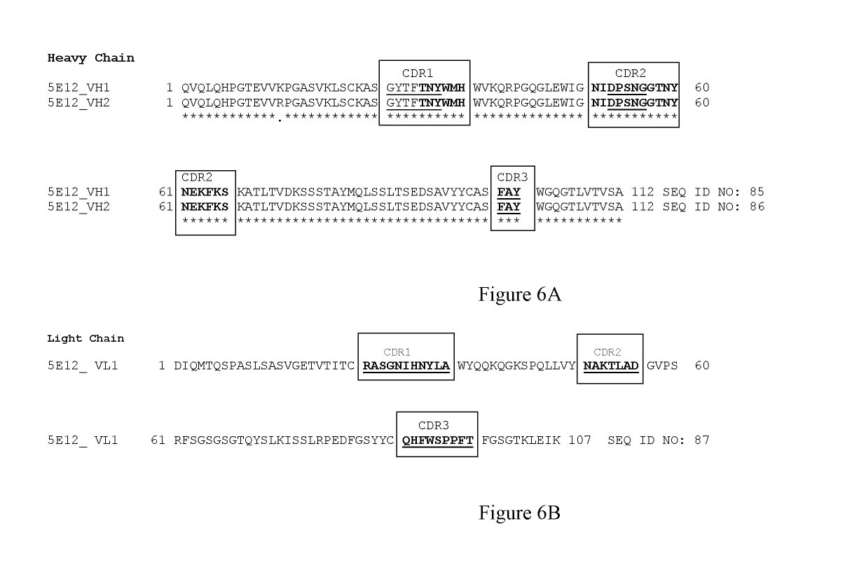

Sequences of the V_{H} and V_{L} of 5E12-B7 were also determined. During this process, two sequences of the V_{H} were determined. When expressed as scFv with the V_{L} of 5E12-B7, both V_{H} were capable of binding to NPY and PYY. The sequences of the V_{H} and V_{L} are shown in FIGS. 6A and 6B.

### Characterization of Affinity and Epitope Mapping for MAb 5E12

Epitope Mapping of MAb 5E12 by ELISA

The epitope specificity of MAb 5E12-B7 was determined by ELISA against plates coated with the synthetic peptides described in Table 3. Initial mapping was performed with peptides based on the last 25 residues of the NPY C-terminus (peptides 1p1-1p18; SEQ ID NOs: 7-24). These peptides contained single alanine substitutions within the last 17 residues of the C terminus. In addition, a set of 10-mer peptides were designed based on the last 23 residues of NPY, with each peptide shifting one residue closer to the C-terminus (peptides 1p19-1p32; SEQ ID NOs: 25-38). All of the peptides carried an amide group at the C-terminus (as do naturally occurring mouse and human NPY).

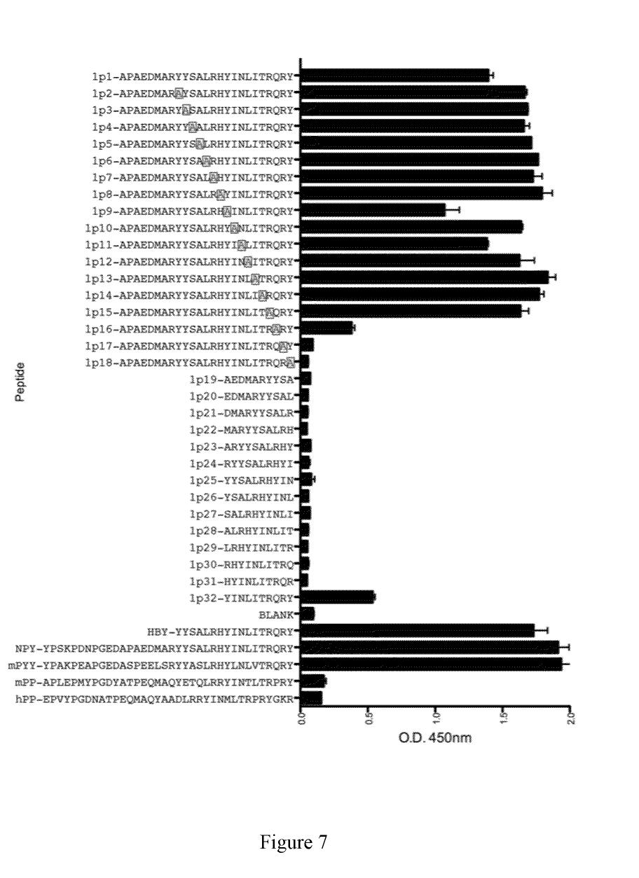

ELISA demonstrated binding of MAb 5E12-B7 to the 25-mer NPY peptides with alanine substitutions at any position up until the last three residues of the C-terminus (FIG. 7). However, the antibody did not detectably and/or significantly bind to 1p16 (SEQ ID NO: 22), 1p17 (SEQ ID NO: 23) or 1p18 (SEQ ID NO: 24) which contained alanine substitutions in the terminal three residues.

ELISA was performed with NPY, mouse PYY, mouse PP and human PP (FIG. 7). MAb 5E12-B7 exhibited maximum binding signal to NPY and mPYY. However, the antibody did not detectably or significantly bind to human or mouse PP.

Epitope mapping was also carried out with a second set of synthetic peptides based on the last 17 residues of NPY (2p1-2p16, Table 3, SEQ ID NOs: 39-54). These peptides contained an increasing chain of alanine residues at the N-terminus with a progressively shrinking C-terminus (based on NPY). This second set of mapping peptides also included a 21-mer with the last 17 residues of NPY followed by four alanines at the C-terminus (2p17, SEQ ID NO: 55). Peptide 2p18 (SEQ ID NO: 56) contained the last 17 residues of mPP with only the third last residue (proline) substituted for the glutamine residue found in NPY and PYY. Peptide 2p19 (SEQ ID NO: 57) contains the last 17 residues of NPY with a carboxyl group at the C terminus instead of the amide group naturally occurring in NPY. Peptide 2p20 (SEQ ID NO: 58) is a 17-mer containing 14 glycine residues followed by the final three residues of NPY at the C terminus.

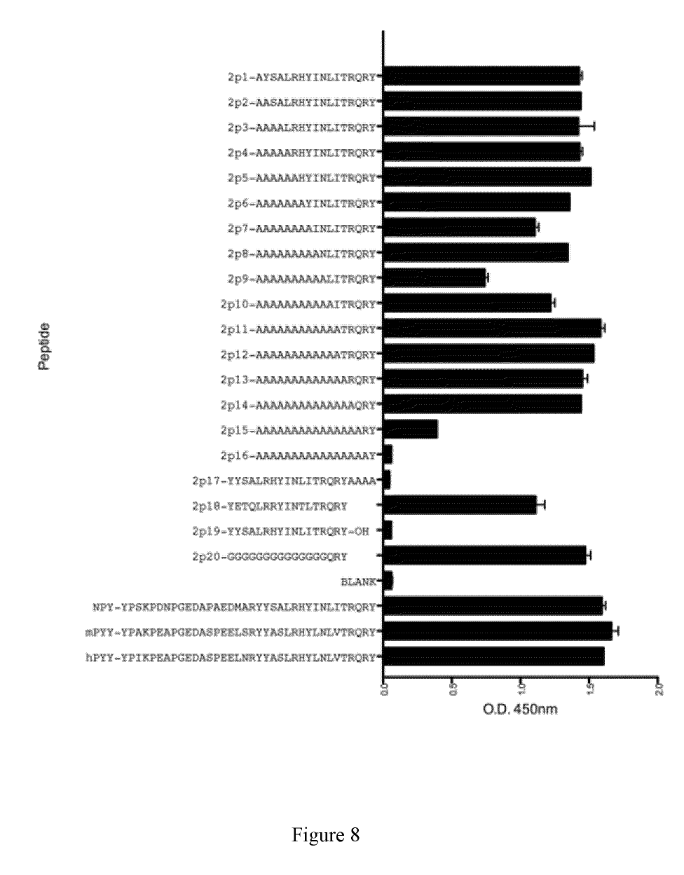

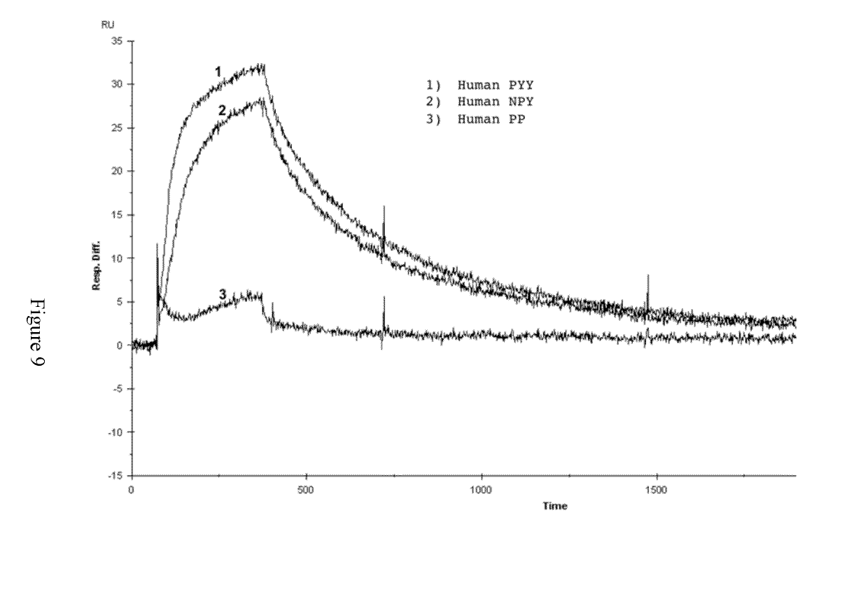

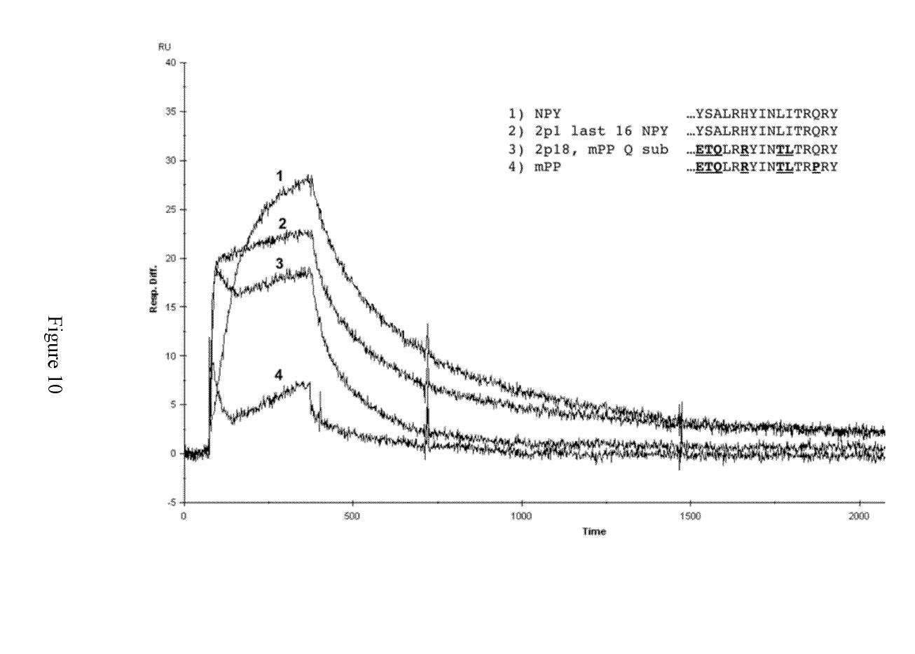

ELISA using MAb 5E12-B7 detected significant and/or detectable levels of binding to peptides 2p1-2p14 (FIG. 8). In contrast, MAb 5E12-B7 did not detectably bind to peptides 2p15 (SEQ ID NO: 53) or 2p16 (SEQ ID NO: 54). ELISA using MAb 5E12-B7 also demonstrated no detectable and/or significant binding to 2p17 (SEQ ID NO: 55) or peptide 2p19 (SEQ ID NO: 57). These data indicate that an amidated tyrosine residue is required at the C-terminus for binding of MAb 5E12-B7. MAb 5E12-B7 also significantly and/or detectably bound to 2p18 (SEQ ID NO: 56) and 2p20 (SEQ ID NO: 58). These data indicate that the sequence QRY wherein the terminal tyrosine is amidated is sufficient for binding of MAb 5E12-B7.

Determination of MAb 5E12-B7 Affinity to NPY, PYY and PP by Biacore

The affinity of MAb 5E12-B7 to NPY, PYY and PP peptides was measured using a Biacore-2000 (GE-healthcare). Biotinylated MAb was loaded onto a single channel of a streptavidin chip, and dilute samples (1 nM-1 μM) of NPY, mPYY, hPYY, mPP and hPP were then run over all four channels of the chip to obtain blank-subtracted readings for peptide on and off-rates. Using this method, significant and/or detectable binding of MAb 5E12-B7 to NPY, mPYY and hPYY was detected. In contrast, no significant and/or detectable binding to mPP and hPP could be detected.

The experiment revealed comparable dissociation rate constants and association rate constants for the NPY and PYY peptides (Table 4).

TABLE 4
K_{D}, k_{d} And kₐ Measurements For MAb 5E12-B7 Obtained By
Fitting Of Association Rate Constants And Dissociation Rate Constants
For Peptides NPY, mPYY, hPYY, mPP And hPP

| Peptide | k_{d} (s⁻¹) | kₐ (M⁻¹ s⁻¹) | K_{D} (M) |
|---|---|---|---|
| NPY | 1.42 × 10⁻³ | 1.49 × 10⁵ | 9.79 × 10⁻⁹ |
| mPYY | 1.81 × 10⁻³ | 4.32 × 10⁵ | 4.67 × 10⁻⁹ |
| hPYY | 1.38 × 10⁻³ | 2.51 × 10⁵ | 5.97 × 10⁻⁹ |

| | |
|---|---|
| mPP | N/A: No binding detected (<8 RU) |
| hPP | N/A: No binding detected (<6 RU) |

Measurement of MAb 5E12-B7 Binding by Biacore

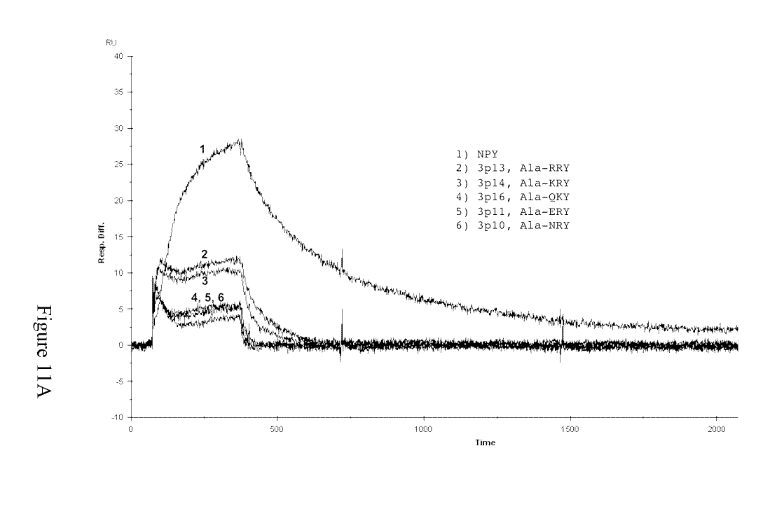

FIG. 10 show the binding of MAb 5E12-B7 to NPY, mouse PP and peptides 2p1 (SEQ ID NO: 39) and 2p18 (SEQ ID NO: 56). These data demonstrate that the sequence PRY-amide when present at the C-terminus of a peptide is not compatible with MAb 5E12-B7 binding. However, mutagenesis of this residue to glutamine restores binding by MAb 5E12-B7. For example, mutation of the proline residue to glutamine in mPP restores binding by MAb 5E12-B7.

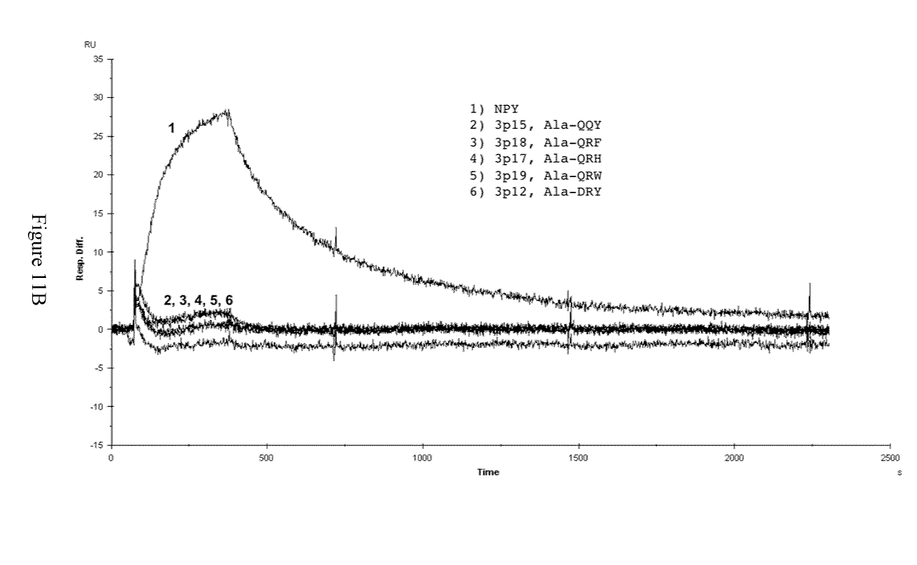

To further characterize the antigenic determinant of MAb 5E12-B7, a series of peptides were used that contain conservative amino acid substitutions at one of the three C-terminal residues (peptides 3p10-3p19, SEQ ID NOs: 68-77). Results are depicted in FIGS. 11A and 11B and can be summarized as follows:
Third last residue from the C-terminus: arginine and lysine substitutions at this position result in detectable and/or significant binding by MAb 5E12-B7.
C-terminal residue: substitutions at this position with residues other then amidated tyrosine (such as amidated phenylalanine, amidated histidine and amidated tryptophan) are not compatible with binding to MAb 5E12-B7. This suggests that tyrosine is an essential determinant for the binding of MAb 5E12-B7.

Cross-Reactivity

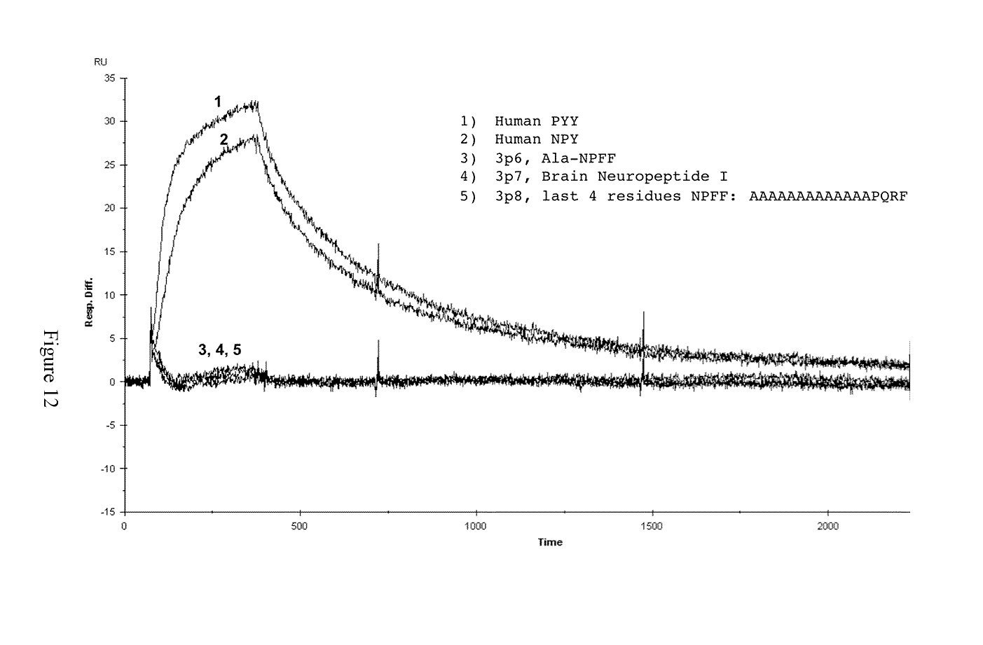

An additional set of peptides was designed to further test the specificity of MAb 5E12-B7 using the Biacore system (FIG. 12). Peptides 3p6 (SEQ ID NO: 64), 3p7 (SEQ ID NO: 65) and 3p8 (SEQ ID NO: 66) which are derived from other neuropeptides, were synthesized for this purpose. However, no detectable and/or significant binding was observed to these peptides derived from NPFF or brain neuropeptide I. These data further demonstrate the specificity of MAb 5E12-B7 for NPY and PYY signaling peptides only.

Determination of MAb 5E12-B7 Affinity to NPY, PYY and PP by Octet

Affinity parameters of MAb 5E12-B7 were measured against peptides NPY, mPYY, hPYY, mPP and hPP using an Octet Red system (ForteBio). Streptavadin sensors were loaded with biotinylated MAb 5E12-B7. Association and dissociation rates were measured by incubation of the sensors in wells containing serial dilutions of the peptides, followed by incubation in assay buffer. As with Biacore analysis, detectable and/or significant binding was observed for MAb 5E12-B7 against NPY, mPYY and hPYY at concentrations from 0-243 nM.

Global fitting of the binding curves identified that MAb 5E12-B7 had affinities ranging from 2-5 nM for peptides NPY, mPYY and hPYY (Table 5). This was in excellent agreement with the values for 5E12-B7 by Biacore. In contrast, 5E12-B7 bound to mPP or hPP with at least 100 times lower affinity than it did to NPY, mPYY and hPYY.

TABLE 5
K_{D}, k_{d} And kₐ Measurements For MAb 5E12-B7 Obtained By Global
Fitting Of Association Rate Constants And Dissociation Rate Constants
For Peptides NPY, mPYY, hPYY, mPP And hPP As Measured By Octet

| Peptide | k_{d} (s⁻¹) | kₐ (M⁻¹ s⁻¹) | K_{D} (M) |
|---|---|---|---|
| NPY | 4.29 × 10⁻⁴ | 9.26 × 10⁴ | 4.70 × 10⁻⁹ |
| mPYY | 5.85 × 10⁻⁴ | 1.86 × 10⁵ | 3.19 × 10⁻⁹ |
| hPYY | 3.85 × 10⁻⁴ | 1.79 × 10⁵ | 2.17 × 10⁻⁹ |
| mPP | 5.49 × 10⁻² | 4.28 × 10⁴ | 1.27 × 10⁻⁶ |
| hPP | 5.41 × 10⁻² | 4.49 × 10⁴ | 1.22 × 10⁻⁶ |

### Example 3

### Anti-NPY and PYY Antibody Neutralizes NPY and PYY Signaling

### Methods

Primary osteoblasts were isolated from 10 calvariae of 2-day-old neonatal wild-type mice. After removing blood vessels and connective tissue, calvariae were minced and then sequentially digested for 10 min in modified Eagle's medium type (-MEM) containing 0.1% collagenase and 0.2% dispase for 5 minutes. Cells from 5 fractions of digestion were combined, seeded and expanded for 2 days at 37° C. and 5% CO₂ in -MEM supplemented with fetal bovine serum (FBS), streptomycin, penicillin G and geneticin. After expansion, cells were plated into 6-well plates at a density of 1.66×10⁵/ml. After reaching 60% confluency, medium was changed to starvation medium with 0.5% FBS for 16 hours prior to use. Cells were incubated for 5 or 20 mins with 20 nM NPY only (human NPY #2055 Auspep, Australia), 200 nM PYY/NPY antibody (5E12) only, or pre-mixed 20 nM NPY and 200 nM PYY/NPY antibody (5E12). Cells with no treatment, and with 200 nM isotype only and combination of 20 nM NPY and 200 nM isotype were served as controls. After treatment, the plates were placed on ice, washed with ice-cold PBS, and harvested with 50-100 μl ice-cold RIPA buffer (25 mM TrisHCl pH 7.6, 150 mM NaCl, 1% NP-40, 1% sodium deoxycholate, 0.1% SDS) supplemented with Complete Protease Inhibitor Cocktail tablets (Complete Mini, Roche Diagnostic, Mannheim, Germany). After centrifugation, clear lysate were collected and protein concentrations were measured using microplate spectrophotometer (Spectramax Plus384, Molecular Devices Inc., Silicon Valley, Calif.) according to Bradford method.

Equal amounts of cell lysates (20 μg protein) were resolved by SDS-PAGE and immunoblotted with antibodies against p-ERK1/2 or total ERK1/2 (1:1000 diluted in 1% BSA, Cell Signaling Technology) on a roller at cold room overnight, followed by incubation with 1:2000 secondary antibody (diluted in 5% skim milk, ECLTM Anti-Rabbit IgG, HRP-Linked whole antibody from donkey, Cat# NA934V, GE Healthcare, UK) at room temperature for 1 hour. Immunolabelled bands were quantified by densitometry.

### Results

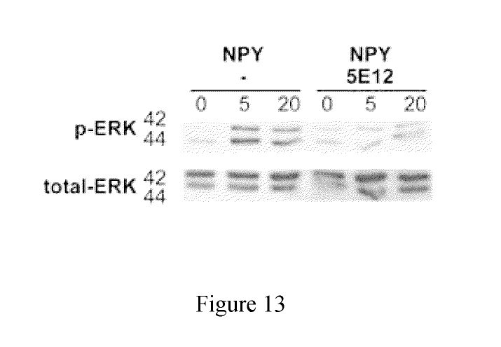

FIG. 13 shows results of analysis of NPY-induced ERK phosphporylation in the presence or absence of antibody MAb 5E12-B7. As shown, MAb 5E12-B7 suppresses the time dependent rise in ERK phosphorylation, indicating that it neutralizes NPY signaling.

### Example 4

### Anti-NPY and PYY Antibody Treats Cancer

### Methods

Mice were injected subcutaneously in the flank with LL2 lung carcinoma cells on day 0. Tumour growth was measured over time as tumour surface area (mm²). Mice received an intraperitoneal injection of 400 μl of PBS supplemented with 40 mg/kg control Ig (cIg) or 5E12 antibody on day 3. This was followed by a subcutaneous injection of 200 μl of PBS supplemented with 20 mg/kg cIg or 5E12 antibody close to the inguinal (tumour draining) lymph node on day 6 and day 10 and once every week thereafter for the remainder of the experiment.

### Results

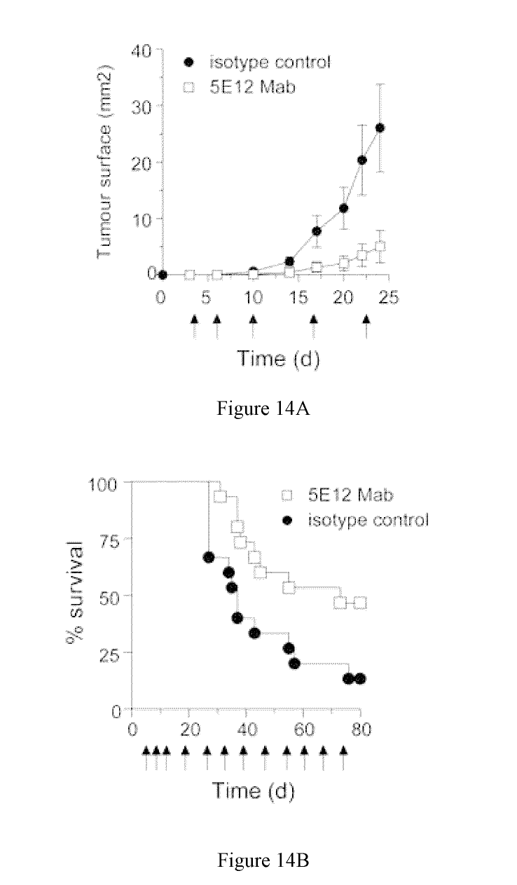

As shown in FIG. 14, treatment of mice, previously administered lung carcinoma cells, with MAb 5E12-B7 significantly suppresses tumor progression/growth (FIG. 14A) and significantly increases survival rate (FIG. 14B).

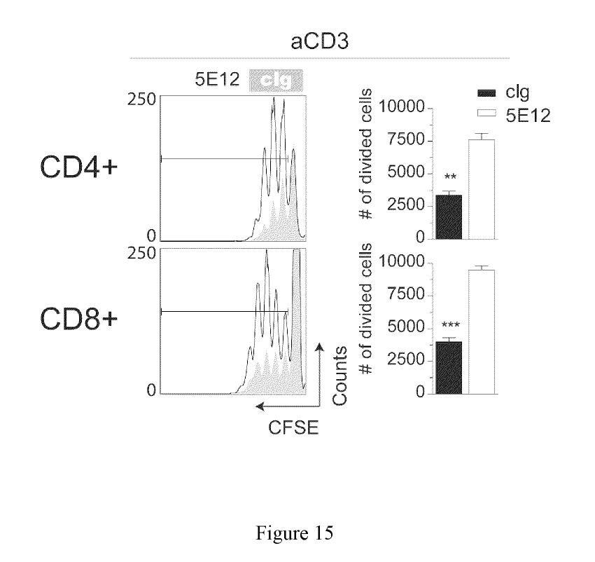

Data presented in FIG. 15 demonstrates that T cells from mice treated with MAb 5E12-B7 have an increased proliferative response, indicating that this antibody is capable of inducing or enhancing an immune response.

### Example 5

### Anti-NPY and PYY Antibody Increases Body Weight and Fat Mass

Mice were treated weekly for 8 weeks with MAb 5E12-B7 at 10 mg/kg from 9 weeks of age onwards.

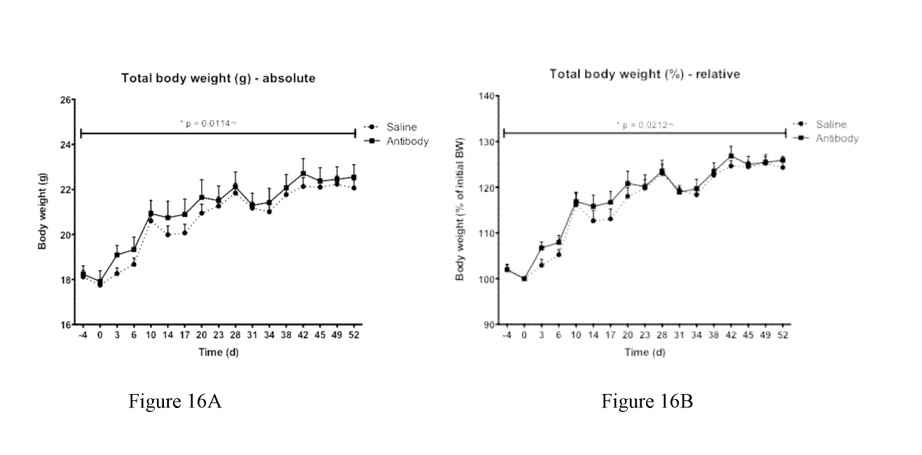

Body weight of mice (n=8) treated with MAb 5E12-B7 or saline were regularly monitored from start of treatment. FIGS. 16A and 16B show an increase in absolute body weight or body weight expressed as percent of initial body weight induced by antibody treatment.

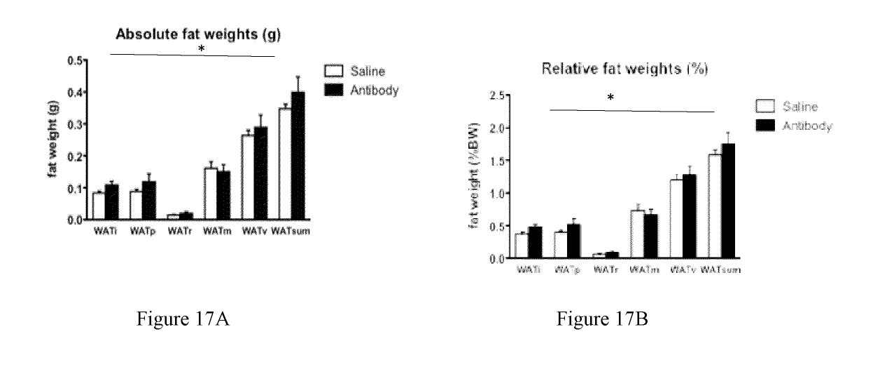

Dissected white adipose tissue (WAT) weights was assessed after 8 weeks of MAb 5E12-B7 or saline treatment when mice reached the age of 17 weeks. FIGS. 17A and 17B show an increase in weights of 4 WAT depots both expressed as absolute as well as in relative values expressed as percent of body weight.

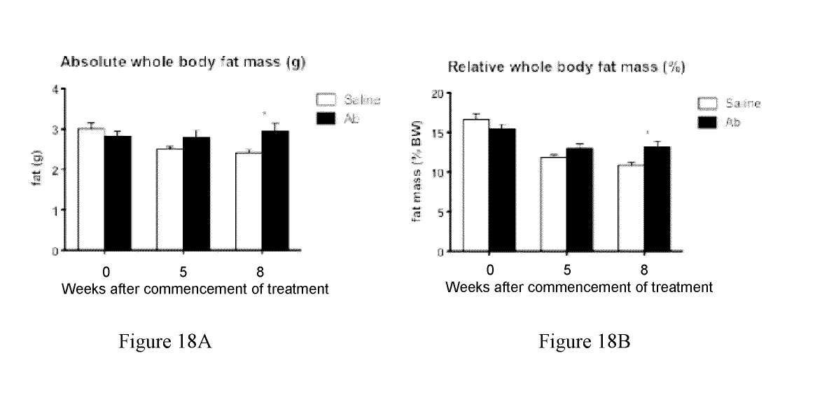

Whole body lean and fat mass was also assessed using dual-energy x-ray absorptiometry (DXA) scan conducted at 0, 5 and 8 weeks following commencement of MAb 5E12-B7 or saline treatment. FIGS. 18A and 18B show an increase in whole body fat measured by DEXA and whole body fat mass expressed as a percent of body weight induced by antibody treatment.

### Example 6

### Humanization of Mouse Anti-NPY/PYY Antibody 5E12

Generation of Humanized 5E12 scFv Genes

Humanized 5E12 V_{H} genes were designed by grafting the 5E12-B7 V_{H} CDRs onto the V_{H} sequences of human germlines IGHV1-46 and IGHV1-69. A humanized 5E12 V_{L} gene was designed by grafting the 5E12-B7 V_{L} CDRs onto the V_{L} sequence of human germlines DPK9. Humanized 5E12-B7 scFv genes h5E12 IGHV1-46/DPK9 and h5E12 IGHV1-69/DPK9 by SOE PCR. The humanized 5E12-B7 scFv genes were cloned into pHENI vector, and transformed TG1 was grown on selective media (TYE, 100 ug/mL ampicillin, 4% glucose). Colonies were picked from the transformation and glycerol stocks were prepared from overnight cultures. Sequence of the two humanized scFv clones was confirmed by sequencing purified PCR products using the appropriate vector primers

### Screening of NPY Binding by Phage ELISA

Overnight cultures were grown from clones transformed with the humanized 5E12-B7 scFv genes pHEN1 vector. Phages were then produced through overnight incubation after rescue with KM13 helper phage. In order to analyze antigen binding, ELISA plates were coated with 500 ng/mL biotin-NPY₂₀₋₃₆ on 10 ug/mL neutravidin; 10 ug/mL neutravidin only; 10 ug/mL Protein A or 10 ug/mL Protein L

ELISA plates were blocked with 5% skim milk/PB ST then incubated with phage culture supernatant which had also been blocked with 5% skim milk/1% Tween (final concentration). Binding of scFv-phage to wells was detected with Peroxidase conjugated anti-M13 secondary antibody.

### Results

ELISA of phage supernatants identified that clones of both humanized 5E12 scFv genes had detectable binding to 500 ng/mL biotin-NPY₂₀₋₃₆ on 10 ug/mL neutravidin plates. No cross-reaction was observed to neutravidin-only plates (blank-subtracted O.D. 450 nm<0.05).

### Example 7

### Identification of Novel NPY/PYY Specific Antibodies by Phage Display Selection

### Phage Selection Campaign

A phage selection campaign was conducted to select human scFvs from the Garvan-2 library (as constructed by Christ) that were capable of binding NPY and PYY peptides. The library was displayed on phage and NPY binders were obtained from the library through four rounds of selection against biotin-NPY₂₀₋₃₆ (Table 6).

TABLE 6
Peptide Sequences

| | | | C |
|---|---|---|---|
| Peptide name | N-terminus | Sequence | terminus |
| biotin-NPY₂₀₋₃₆ | Biotin-Ahx- | YYSALRHYINLITRQRY | —NH₂ |
| | | (SEQ ID NO: 107) | |
| biotin-NPY | Biotin-Ahx- | YPSKPDNPGEDAPAEDMARYYSALRHYINLITRQRY | —NH₂ |
| | | (SEQ ID NO: 108) | |
| biotin-hPYY | Biotin-Ahx- | YPIKPEAPGEDASPEELNRYYASLRHYLNLVTRQRY | —NH₂ |
| | | (SEQ ID NO: 109) | |
| biotin-hPP | Biotin-Ahx- | APLEPVYPGDNATPEQMAQYAADLRRYINMLTRPRY | —NH₂ |
| | | (SEQ ID NO: 110) | |

184 clones were picked from the fourth-round binders and overnight cultures were stored as glycerol stocks (2×96-well plates).

### Monoclonal Screening of NPY Binding by Phage ELISA

Overnight cultures were grown from 176 clones picked from round 4 of the phage selection campaign. Phages were produced through overnight incubation after rescue with KM13 helper phage. In order to analyze antigen binding, ELISA plates were coated with 500 ng/mL biotin-NPY₂₀₋₃₆ on 10 ug/mL neutravidin; 500 ng/mL biotin-NPY₂₀₋₃₆ on 10 ug/mL streptavidin; 10 ug/mL neutravidin only or 10 ug/mL streptavidin only

The ELISA plates were blocked with 5% skim milk/PB ST then incubated with phage culture supernatant which had also been blocked with 5% skim milk/1% Tween (final concentration). Binding of scFv-phage to wells was detected with peroxidase conjugated anti-M13.

### Results

Binding to biotin-NPY₂₀₋₃₆ was observed for 172/176 (98%) of the clones regardless of whether the peptide was coupled to streptavidin or neutravidin (O.D. 450 nm>0.5). Of the biotin-NPY₂₀₋₃₆ binders, only 12/172 (7%) were found to cross-react to plates coated with streptavidin only (O.D. 450 nm>0.5). None of the clones cross-reacted to plates coated with neutravidin only.

Monoclonal Screening of NPY Binding by Soluble scFv ELISA

IPTG-induced soluble scFv cultures were produced from all 176 of the round 4 clones previously screened by phage ELISA. In order to analyze antigen binding, ELISA plates were coated with 1 ug/mL biotin-NPY₂₀₋₃₆ on 10 ug/mL streptavidin or 10 ug/mL or 10 ug/mL streptavidin only.

Plates were then blocked with 5% skim milk/PB ST and incubated with supernatant from the IPTG-induced cultures. Binding of soluble scFv to wells was detected with peroxidase conjugated anti-cMyc.

### Results

Binding was detected for several clones. Binding was also detected for positive control (mouse 5E12-B7 scFv PSE2), but not for an irrelevant scFv. No cross-reactivity to streptavidin-only wells was observed in any of the clones (blank-subtracted O.D. 450 nm<0.06). The 32 clones with the highest binding to biotin-NPY₂₀₋₃₆ (Blank-subtracted O.D. 450 nm range 0.195-0.51) were selected for sequencing.

### Sequencing of Biotin-NPY₂₀₋₃₆ Binding Clones:

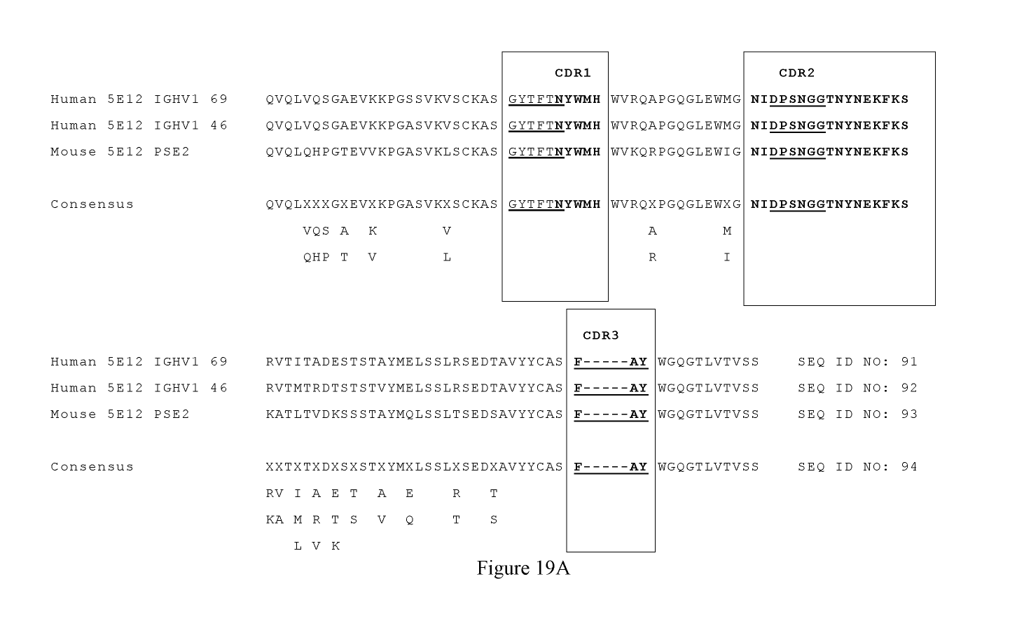

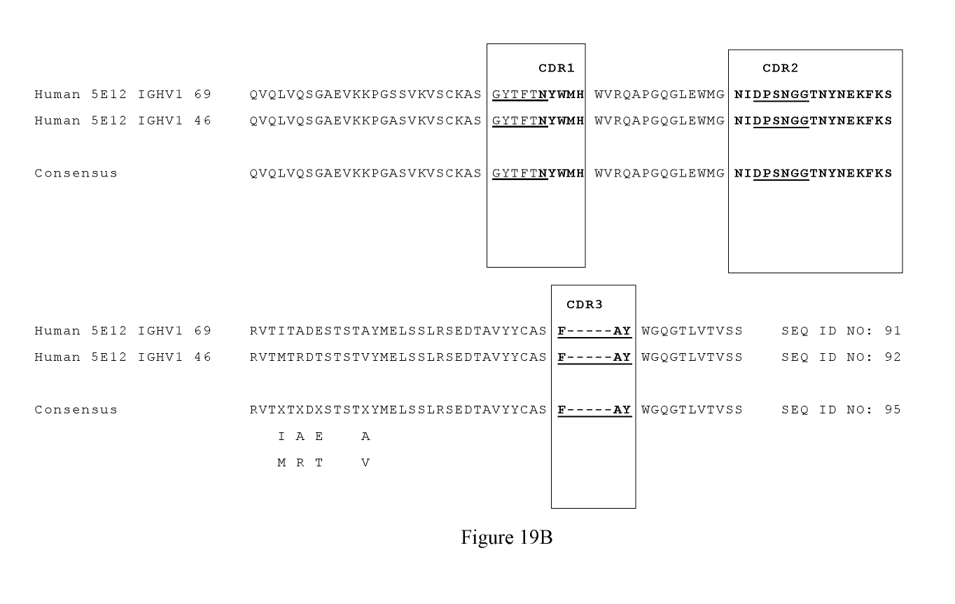

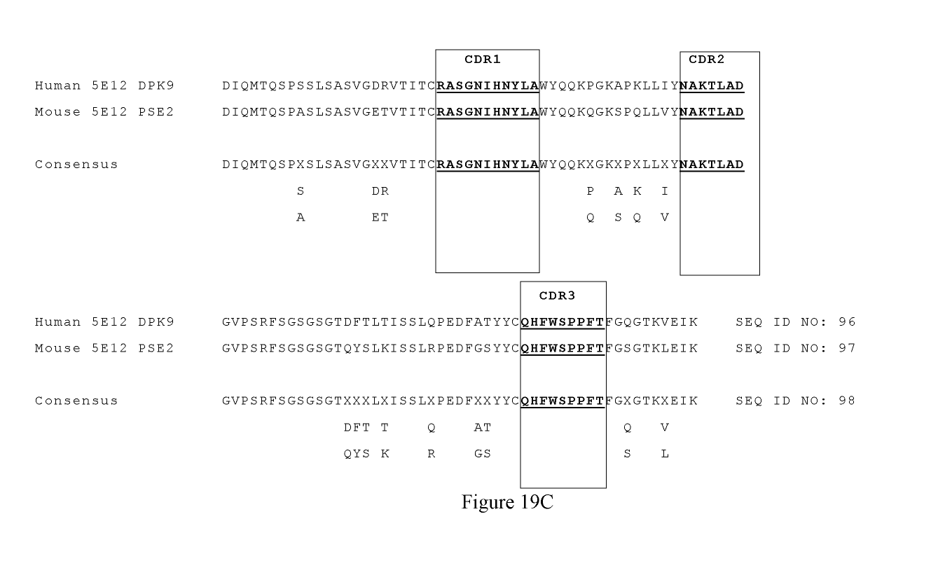

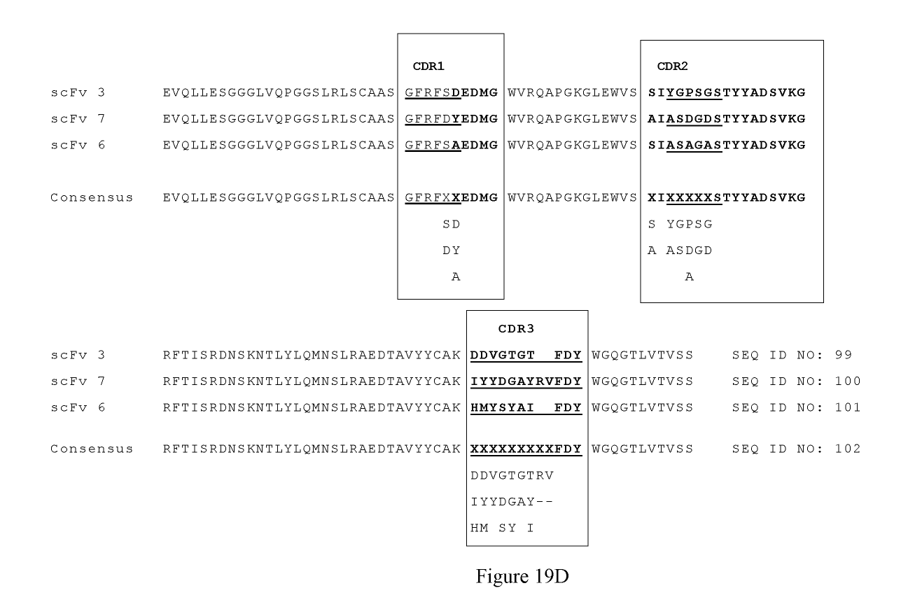

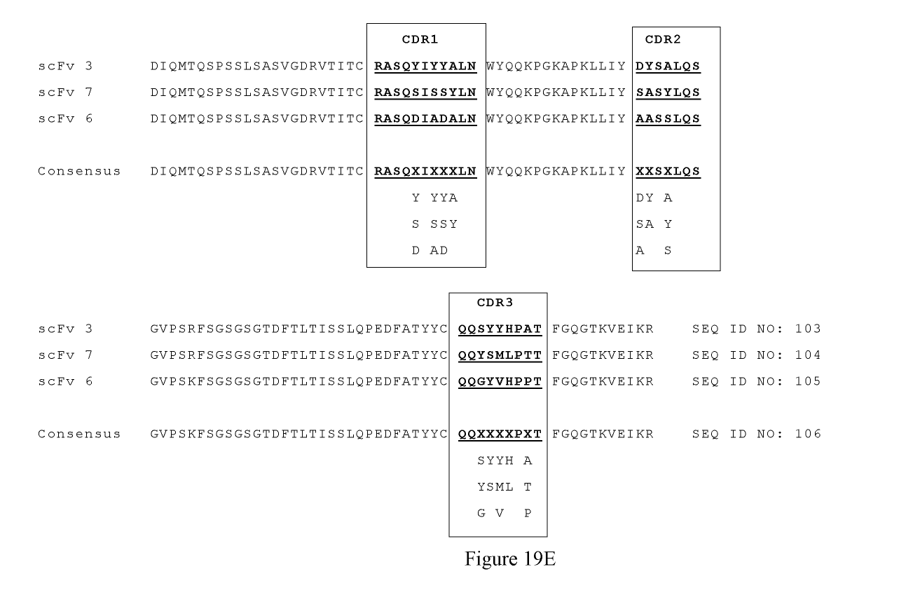

Sequence analysis of the 32 clones with highest apparent binding to biotin-NPY₂₀₋₃₆ in soluble scFv ELISA yielded three unique scFv genes (Table 2). These genes were named scFv-3, scFv-6 and scFv-7. Translation of the genes revealed unique V_{H} and V_{L} CDR sequences (FIG. 19D).

### Example 8

### Determination of Affinity Against NPY, hPYY and hPP for Human Antibodies and Humanized 5E12 Variants

### Method

The scFv genes scFv-3 (human); scFv-6 (human); scFv-7 (human); humanized 5E12 variant h5E12 IGHV1-46/DPK9; humanized 5E12 variant h5E12 IGHV1-69/DPK9 and mouse 5E12-B7 scFv PSE2 (expressed using pHEN1 vector transformed into HB2151 strain) were cloned into pET12a vector which was used to transform BL21-Gold for large scale soluble scFv expression

Soluble scFv expression was induced with 1 μM IPTG. Soluble scFv was purified from culture supernatants and periplasmic extracts using protein L sepharose.

Affinity of the purified scFv for biotin-NPY, biotin-hPYY and biotin-hPP was determined using a Blitz system (ForteBio). Streptavidin Biosensors were blocked for 1 hr with 1% BSA in PBS. Biosensors were then coupled for 120 seconds with 1 μM solutions of biotinylated peptide followed by a 30 second baseline measurement. The sensors were then transferred to varying dilutions of purified scFv and association was measured for 300 seconds. The sensors were then transferred to PBS and dissociation was measured for 900 seconds. Global fits were determined for association and dissociation curves at two concentrations of each scFv against each of the peptides. All curves were referenced with peptide-only and scFv-only runs.

### Results

Affinity results for the six scFvs listed above are in Table 7.

TABLE 7
Binding affinity of mouse, human and humanized antibodies
to NPY and PYY (all in scFv format; ForteBio Blitz system).

| | Peptide | KD | ka | kd |
|---|---|---|---|---|
| 5E12 (mouse) | Biotin-NPY | 2.214 × 10⁻⁸ | 4.344 × 10⁴ | 9.618 × 10⁻⁴ |
| | Biotin-hPYY | 3.294 × 10⁻⁸ | 3.318 × 10⁴ | 1.093 × 10⁻³ |
| | Biotin-hPP | N/A no binding detected | | |
| | | (max signal <0.2 nm) | | |
| scFv-3(human) | Biotin-NPY | 1.858 × 10⁻⁶ | 6.171 × 10³ | 1.147 × 10⁻² |
| | Biotin-hPYY | 1.915 × 10⁻⁶ | 9.409 × 10³ | 1.802 × 10⁻² |
| | Biotin-hPP | N/A no binding detected | | |
| | | (max signal <0.2 nm) | | |
| scFv-6(human) | Biotin-NPY | 1.771 × 10⁻⁷ | 3.233 × 10³ | 5.725 × 10⁻⁴ |
| | Biotin-hPYY | 3.240 × 10⁻⁶ | 1.808 × 10³ | 5.857 × 10⁻³ |
| | Biotin-hPP | N/A no binding detected | | |
| | | (max signal <0.2 nm) | | |
| scFv-7(human) | Biotin-NPY | 9.254 × 10⁻⁶ | 7.892 × 10³ | 7.303 × 10⁻² |
| | Biotin-hPYY | 3.483 × 10⁻⁵ | 9.352 × 10³ | 3.257 × 10⁻¹ |
| | Biotin-hPP | N/A no binding detected | | |
| | | (max signal <0.2 nm) | | |
| h5E12 IGHV1- | Biotin-NPY | 9.050 × 10⁻⁷ | 6.327 × 10³ | 5.726 × 10⁻³ |
| 46/DPK9 | Biotin-hPYY | 1.036 × 10⁻⁶ | 6.938 × 10³ | 7.187 × 10⁻³ |
| (humanized) | Biotin-hPP | N/A no binding detected | | |
| | | (max signal <0.2 nm) | | |
| h5E12 IGHV1- | Biotin-NPY | 1.423 × 10⁻⁷ | 1.953 × 10⁴ | 2.779 × 10⁻³ |
| 69/DPK9 | Biotin-hPYY | 1.396 × 10⁻⁷ | 2.689 × 10⁴ | 3.753 × 10⁻³ |

| | | |
|---|---|---|
| (humanized) | Biotin-hPP | N/A no binding detected |
| | | (max signal <0.2 nm) |

### Example 9

### Anti-NPY and PYY Antibody Treats Breast Cancer

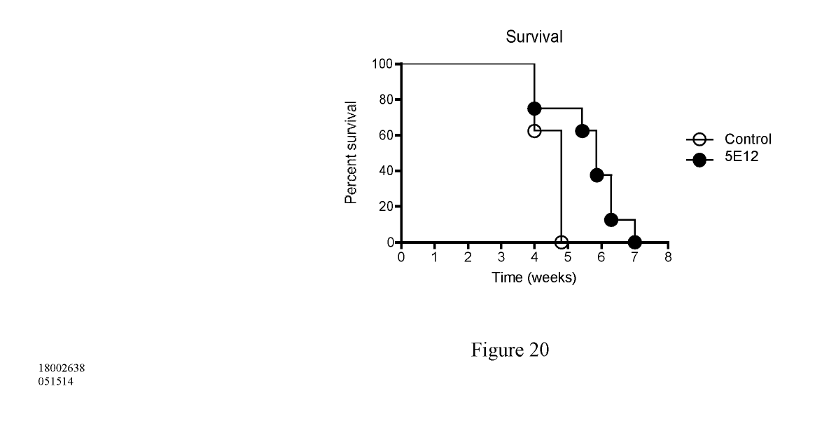

About 1×10⁵ 4 T1 breast cancer cells expressing luciferase were injected into mammary fat pad of BALB/c female mice. After 48 hours 14 (out of 16) mice had detectable tumours (as determined by D-luciferin imaging). The mice (n=14) were treated once IP with 5E12 monoclonal antibody (mIgG2a isotype) or isotype control at 40 mg/kg. This was followed by IP dosing at 20 mg/kg once a week. As can be seen in FIG. 20, treatment with 5E12 monoclonal antibody results in significantly increased survival (p=0.01).

What is claimed is:

1. An isolated or recombinant neuropeptide Y (NPY) and peptide YY (PYY)-binding protein comprising an antibody variable region, wherein the protein specifically binds to NPY and PYY.

2. The NPY and PYY-binding protein of claim 1, which binds to NPY and/or PYY with an affinity at least 100 times greater than it binds to pancreatic polypeptide (PP).

3. The NPY and PYY-binding protein of claim 1, wherein the protein does not detectably bind to pancreatic polypeptide (PP).

4. The protein of claim 1, which binds to an epitope comprising in amino-carboxy order:
(i) a positively charged amino acid, a positively charged amino acid and an amidated tyrosine; or
(ii) an amino acid structurally related to glutamine, a positively charged amino acid and an amidated tyrosine; or
(iii) XRY-amide, wherein X is Q or K or R (SEQ ID NO: 79).

5. The protein of any one of claims 1 to 4 which binds to an epitope comprising the sequence QRY-amide at its C terminus (SEQ ID NO: 80).

6. An isolated or recombinant neuropeptide Y (NPY) and peptide YY (PYY)-binding protein comprising an antibody variable region, wherein the protein specifically binds to NPY and PYY, wherein the protein binds to an epitope comprising the sequence QRY-amide (SEQ ID NO: 80) at its C terminus.

7. The NPY and PYY-binding protein of any one of claims claim 4 to 6, which does not significantly or detectably bind to an epitope comprising the sequence PRY-amide at its C terminus (SEQ ID NO: 81).

8. The NPY and PYY-binding protein of claim 7, wherein the protein binds to an epitope comprising the sequence QRY-amide at its C terminus (SEQ ID NO: 80) with at least 100 times greater affinity than it does to an epitope comprising the sequence PRY-amide at its C terminus (SEQ ID NO: 81).

9. The NPY and PYY-binding protein of any one of claims 4 to 8, which binds with significantly greater affinity to an epitope comprising an amidated C-terminal tyrosine than it does to an epitope in which the C-terminal tyrosine is substituted with a non-polar amino acid.

10. The NPY and PYY-binding protein of any one of claims 4 to 9, which does not detectably bind to an epitope in which the amidated C-terminal tyrosine is substituted with a non-polar amino acid.

11. The NPY and PYY-binding protein of any one of claims 1 to 10, wherein the protein does not detectably or significantly bind to an epitope comprising the sequence FMRF-amide (SEQ ID NO: 82) or FLFQPQRF-amide (SEQ ID NO: 83) or QRF-amide (SEQ ID NO: 84).

12. The NPY and PYY-binding protein of claim 11, wherein the protein does not bind to a polypeptide comprising the sequence FMRF-amide (SEQ ID NO: 82) or FLFQPQRF-amide (SEQ ID NO: 83) or QRF-amide (SEQ ID NO: 84) at its C-terminus.

13. The NPY and PYY-binding protein of any one of claims 1 to 12, wherein the protein neutralizes NPY and PYY-mediated signaling in a cell.

14. The NPY and PYY-binding protein of claim 13, wherein the NPY and PYY-mediated signaling in a cell is ERK phosphorylation.

15. The NPY and PYY-binding protein of any one of claims 1 to 3, wherein the variable domain competitively inhibits binding of one or more of the following to NPY and/or PYY:
(i) an antibody comprising a heavy chain variable region (VH) comprising a sequence set forth in SEQ ID NO: 85 or 86 and a light chain variable region (VL) comprising a sequence set forth in SEQ ID NO: 87;
(ii) an antibody comprising a VH comprising a sequence set forth in SEQ ID NO: 91 and a VL comprising a sequence set forth in SEQ ID NO: 96;
(iii) an antibody comprising a VH comprising a sequence set forth in SEQ ID NO: 92 and a VL comprising a sequence set forth in SEQ ID NO: 96;
(iv) an antibody comprising a VH comprising a sequence set forth in SEQ ID NO: 99 and a VL comprising a sequence set forth in SEQ ID NO: 103;
(v) an antibody comprising a VH comprising a sequence set forth in SEQ ID NO: 100 and a VL comprising a sequence set forth in SEQ ID NO: 104; and/or
(vi) an antibody comprising a VH comprising a sequence set forth in SEQ ID NO: 101 and a VL comprising a sequence set forth in SEQ ID NO: 105.

16. The NPY and PYY-binding protein of any one of claims 1 to 3, wherein the variable domain binds to the same epitope as one or more of the following:
(i) an antibody comprising a heavy chain variable region (VH) comprising a sequence set forth in SEQ ID NO: 85 or 86 and a light chain variable region (VL) comprising a sequence set forth in SEQ ID NO: 87;
(ii) an antibody comprising a VH comprising a sequence set forth in SEQ ID NO: 91 and a VL comprising a sequence set forth in SEQ ID NO: 96;
(iii) an antibody comprising a VH comprising a sequence set forth in SEQ ID NO: 92 and a VL comprising a sequence set forth in SEQ ID NO: 96;
(iv) an antibody comprising a VH comprising a sequence set forth in SEQ ID NO: 99 and a VL comprising a sequence set forth in SEQ ID NO: 103;
(v) an antibody comprising a VH comprising a sequence set forth in SEQ ID NO: 100 and a VL comprising a sequence set forth in SEQ ID NO: 104; and/or
(vi) an antibody comprising a VH comprising a sequence set forth in SEQ ID NO: 101 and a VL comprising a sequence set forth in SEQ ID NO: 105.

17. An isolated or recombinant neuropeptide Y (NPY) and peptide YY (PYY)-binding protein comprising an antibody variable region, wherein (i) the protein specifically binds to NPY and PYY; (ii) the protein neutralizes NPY and PYY signaling; and (iii) the protein does not significantly and detectably bind to any one or more of SEQ ID NOs: 5, 6, 22-37, 53-55, 57, 59, 60, 63-67, 70, 75, 76 or 77.

18. The NPY and PYY-binding protein of any one of claims 1 to 17, comprising a heavy chain variable region (VH) and a light chain variable region (VL), wherein the VH and VL bind to form a Fv that specifically binds to NPY and PYY.

19. An isolated neuropeptide Y (NPY) and peptide YY (PYY)-binding protein comprising:
(i) a heavy chain variable region (VH) comprising the CDRs of a VH comprising a sequence set forth in SEQ ID NO: 85 or 86; and
(ii) a light chain variable region (VL) comprising the CDRs of a VL comprising a sequence set forth in SEQ ID NO: 87,
wherein the VH and VL bind to form a Fv that specifically binds to NPY and PYY.

20. An isolated or recombinant NPY and PYY-binding protein comprising a heavy chain variable region (VH) comprising the complementarity determining regions (CDRs) of a VH comprising a sequence set forth in SEQ ID NO: 102 and a light chain variable region (VL) comprising the CDRs of a VL comprising a sequence set forth in SEQ ID NO: 106,
wherein the VH and VL bind to form a Fv that specifically binds to NPY and PYY.

21. An isolated or recombinant NPY and PYY-binding protein comprising:
(i) a heavy chain variable region (VH) comprising the complementarity determining regions (CDRs) of a VH comprising a sequence set forth in SEQ ID NO: 99 and a light chain variable region (VL) comprising the CDRs of a VL comprising a sequence set forth in SEQ ID NO: 103;
(ii) a VH comprising the CDRs of a VH comprising a sequence set forth in SEQ ID NO: 100 and a VL comprising the CDRs of a VL comprising a sequence set forth in SEQ ID NO: 104; or
(iii) a VH comprising the CDRs of a VH comprising a sequence set forth in SEQ ID NO: 101 and a VL comprising the CDRs of a VL comprising a sequence set forth in SEQ ID NO: 105;
wherein the VH and VL bind to form a Fv that specifically binds to NPY and PYY.

22. An isolated or recombinant neuropeptide Y (NPY) and peptide YY (PYY)-binding protein comprising a heavy chain variable region (VH) comprising a sequence set forth in SEQ ID NO: 85 or 86 and/or a light chain variable region (VL) comprising a sequence set forth in SEQ ID NO: 87 or a chimeric, deimmunized, CDR grafted, humanized or synhumanized form thereof,
wherein the VH and VL bind to form a Fv that specifically binds to NPY and PYY.

23. The protein according to claim 22, which is humanized and comprises a VH comprising a sequence set forth in SEQ ID NO: 94 or 95 and/or a VL comprising a sequence set forth in SEQ ID NO: 98.

24. The protein according to claim 23, which comprises one of the following:
(i) a VH comprising a sequence set forth in SEQ ID NO: 91 and/or a VL comprising a sequence set forth in SEQ ID NO: 96; and/or
(ii) the humanized protein comprises a VH comprising a sequence set forth in SEQ ID NO: 92 and/or a VL comprising a sequence set forth in SEQ ID NO: 96.

25. An isolated or recombinant NPY and PYY-binding protein comprising one of the following:
(i) a heavy chain variable region (VH) comprising a sequence set forth in SEQ ID NO: 99 and/or a light chain variable region (VL) comprising a sequence set forth in SEQ ID NO: 103;
(ii) a VH comprising a sequence set forth in SEQ ID NO: 100 and/or a VL comprising a sequence set forth in SEQ ID NO: 104; or
(iii) a VH comprising a sequence set forth in SEQ ID NO: 101 and/or a VL comprising a sequence set forth in SEQ ID NO: 105.

26. The NPY and PYY-binding protein of any one of claims 18 to 25, wherein the VH and the VL are in a single polypeptide chain.

27. The NPY and PYY-binding protein of claim 26, which is:
(i) a single chain Fv fragment (scFv);
(ii) a dimeric scFv (di-scFv); or
(iii) at least one of (i) and/or (ii) linked to a Fc or a heavy chain constant domain (CH) 2 and/or CH3.

28. The NPY and PYY-binding protein of any one of claims 18 to 25, wherein the VL and VH are in separate polypeptide chains.

29. The NPY and PYY-binding protein of claim 28, which is:
(i) a diabody;
(ii) a triabody;
(iii) a tetrabody;
(iv) a Fab;
(v) a F(ab′)2;
(vi) a Fv; or
(iv) one of (i) to (vi) linked to a Fc or a heavy chain constant domain (CH) 2 and/or CH3.

30. The NPY and PYY-binding protein of claim 28, which is an antibody.

31. The NPY and PYY-binding protein of claim 30, which is an antibody comprising one of the following
(i) a heavy chain variable region (VH) comprising a sequence set forth in SEQ ID NO: 85 or 86 and/or a light chain variable region (VL) comprising a sequence set forth in SEQ ID NO: 87 or a chimeric, deimmunized, CDR grafted, humanized or synhumanized form of the antibody;
(ii) a VH comprising a sequence set forth in SEQ ID NO: 94 or 95 and a VL comprising a sequence set forth in SEQ ID NO: 98;
(iii) a VH comprising a sequence set forth in SEQ ID NO: 91 and a VL comprising a sequence set forth in SEQ ID NO: 96;
(iv) a VH comprising a sequence set forth in SEQ ID NO: 92 and/or a VL comprising a sequence set forth in SEQ ID NO: 96;
(v) a VH comprising a sequence set forth in SEQ ID NO: 102 and/or a VL comprising a sequence set forth in SEQ ID NO: 106;
(vi) a VH comprising a sequence set forth in SEQ ID NO: 99 and/or a VL comprising a sequence set forth in SEQ ID NO: 103;
(vii) a VH comprising a sequence set forth in SEQ ID NO: 100 and/or a VL comprising a sequence set forth in SEQ ID NO: 104; or
(viii) a VH comprising a sequence set forth in SEQ ID NO: 101 and/or a VL comprising a sequence set forth in SEQ ID NO: 105.

32. A composition comprising the NPY and PYY-binding protein of any one of claims 1 to 31 and a pharmaceutically acceptable carrier.

33. A method of treating or preventing a NPY and/or PYY-mediated condition in a subject in need thereof, the method comprising administering the NPY and PYY-binding protein of any one of claims 1 to 31 or the composition of claim 32 to the subject.

34. The method of claim 28, wherein the NPY and/or PYY-mediated condition is anorexia or a wasting condition.

35. A method of treating or preventing cancer in a subject, the method comprising inhibiting NPY and PYY signaling in cells of the subject.

36. The method of claim 35 comprising administering to the subject one or more compounds that bind to NPY and/or PYY to thereby inhibit NPY and PYY signaling in cells of the subject.

37. The method of claim 36, wherein the compound(s) is/are a protein(s) comprising an antibody variable region.

38. The method of claim 36 or 37 comprising administering a protein comprising an antibody variable region that binds to and inhibits PYY and a protein comprising an antibody variable region that binds to and inhibits NPY.

39. The method of claim 36 or 37 comprising administering a single protein that binds to and inhibits NPY and PYY.

40. The method of claim 39 comprising administering a protein comprising an antibody variable region that binds to and inhibits PYY and an antibody variable region that binds to and inhibits NPY.

41. The method of claim 40, comprising administering a protein comprising an antibody variable region that binds to NPY and PYY.

42. The method of claim 41, comprising administering the protein of any one of claims 1 to 32 or the composition of claim 33.

43. The method of any one of claims 35 to 42, wherein the cancer is not neuroblastoma.

44. The method of claim any one of claims 35 to 42, wherein the cancer is selected from the group consisting of an adenocarcinoma, a squamous cell carcinoma, a digestive/gastrointestinal cancer, an eye cancer, a musculoskeletal cancer, a breast cancer, a genitourinary cancer, a germ cell cancer, a head and neck cancer, a hematologic/blood cancer, a respiratory cancer, a skin cancer, an AIDS-related malignancy or a genealogic cancer.

45. The method of any one of claims 35 to 44, wherein the cancer expresses a NPY receptor responsive to NPY and PYY.

46. The method of claim 45, wherein the cancer expresses a Y1 receptor and/or a Y2 receptor and/or a Y5 receptor.

47. The method of claim 45 or 46, additionally comprising detecting expression of the Y receptor(s).

48. The method of any one of claims 35 to 47, comprising administering an amount of the compound sufficient to induce or enhance an immune response against the cancer in the subject.

49. The method of claim 48, wherein the immune response is a T cell response.

50. The method of any one of claims 35 to 49 additionally comprising administering a further compound to treat the cancer or exposing the subject to radiation therapy.

51. The method of any one of claims 35 to 50, wherein the subject additionally suffers from a wasting condition.

52. A method for inducing or enhancing an immune response in a subject, the method comprising inhibiting NPY and PYY signaling in cells of the subject.

53. The method of claim 52, wherein the subject suffers from cancer and the immune response is against the cancer or a cell thereof.

54. The method of claim 52 or 53 comprising administering to the subject one or more compounds that bind to NPY and/or PYY to thereby inhibit NPY and PYY signaling in cells of the subject.

55. The method of any one of claims 52 to 53, comprising administering the protein of any one of claims 1 to 31 or the composition of claim 32.
